(19) United States
(12) Patent Application Publication
(10) Pub. No.: US 2026/0308242 A1
(43) Pub. Date: Oct. 8, 2026

(54) COMPOSTIONS OF MULTISPECIFIC ANTIGEN BINDING POLYPEPTIDES AND METHODS OF USE

(71) Applicant: RevOpsis Therapeutics, Inc., Tampa, FL (US)

(72) Inventors: Ramanath BHANDARI, Springfield, IL (US); Li XU, Cupertino, CA (US); Ramesh BHATT, Belmont, CA (US); Ram Prashanth PALANKI, Verona, WI (US)

(21) Appl. No.: 19/577,884

(22) Filed: Mar. 25, 2026

Related U.S. Application Data
(60) Provisional application No. 63/778,102, filed on Mar. 26, 2025.
(60) Provisional application No. 63/799,174, filed on May 2, 2025.
(60) Provisional application No. 63/803,269, filed on May 9, 2025.

(51) Int. Cl. C07K 16/22; A61K 39/00; A61P 27/02

(52) U.S. Cl. CPC C07K 16/22; A61P 27/02; A61K 2039/505; C07K 2317/31; C07K 2317/565

(57) ABSTRACT

Described herein are ANG-2 binding antibodies and multispecific antigen binding polypeptides comprising an ANG-2 binding moiety, a VEGF-A binding moiety, and/or a VEGF-C binding moiety. Described herein are pharmaceutical compositions comprising the ANG-2 binding antibodies or multispecific antigen binding polypeptides described herein. Also described herein are methods of treating an ocular disease in an individual comprising administering the ANG-2 binding antibodies or multispecific antigen binding polypeptides described herein.

### CROSS-REFERENCE

This application claims the benefit of U.S. Provisional Application No. 63/778,102, filed Mar. 26, 2025, U.S. Provisional Application No. 63/799,174, filed May 2, 2025, and U.S. Provisional Application No. 63/803,269, filed May 9, 2025, which applications are incorporated herein by reference in their entireties.

### STATEMENT AS TO FEDERALLY SPONSORED RESEARCH

This invention was made with government support under 1R44EY035584-01A0 Small Business Innovation Research (SBIR) awarded by the National Institutes of Health. The government has certain rights in the invention.

### SEQUENCE LISTING

The instant application contains a Sequence Listing which has been submitted electronically in XML file format and is hereby incorporated by reference in its entirety. Said XML copy, created on Apr. 2, 2026, is named 63209-729_201_SL.xml and is 102,836 bytes in size.

### SUMMARY

Described herein are trispecific antigen binding polypeptides targeting VEGF-A, ANG-2, and VEGF-C. Current therapies targeting only one or two of these targets, e.g. anti-VEGF-A monotherapy for the treatment of ocular diseases, require frequent ocular injections and lead to inconsistent outcomes. Trispecific antigen binding polypeptides described herein possess certain advantages, such as high binding affinities to multiple targets to enhance biological activity, reduced risk of immunogenicity, reduced off-target effects (such as minimal inhibition of ANG-1 binding to Tie2 receptor), and improved and scalable cost-efficient manufacturability.

Described herein in one aspect, is Angiopoietin-2 (ANG-2) binding antibodies or antigen binding fragment thereof comprising: an ANG-2 heavy chain variable region paired to a light chain polypeptide, wherein the ANG-2 heavy chain variable region comprises: a heavy chain complementarity determining region 1 (HCDR1) comprising the amino acid sequence set forth in any one of SEQ ID NOs: 13, 41, 44, and 47; a heavy chain complementarity determining region 2 (HCDR2) comprising the amino acid sequence set forth in any one of SEQ ID NOs: 14, 42, 45, and 48; and a heavy chain complementarity determining region 3 (HCDR3) comprising the amino acid sequence set forth in any one of SEQ ID NOs: 15, 43, 46, and 49.

In some embodiments, the ANG-2 heavy chain variable region comprises an amino acid sequence comprising at least 85%, 90%, 95%, 97%, 98%, or 99% identity to the amino acid sequence set forth in SEQ ID NO: 11. In some embodiments, the ANG-2 heavy chain variable region comprises the amino acid sequence set forth in SEQ ID NO: 11. In some embodiments, the ANG-2 binding moiety comprises an amino acid sequence comprising at least 85%, 90%, 95%, 97%, 98%, or 99% identity to the amino acid sequence set forth in SEQ ID NO: 12. In some embodiments, the ANG-2 binding moiety comprises the amino acid sequence set forth in SEQ ID NO: 12. In some embodiments, the light chain polypeptide is non-covalently associated with the ANG-2 heavy chain variable region. In some embodiments, the light chain polypeptide is covalently associated with the ANG-2 heavy chain variable region. In some embodiments, the light chain polypeptide is covalently associated with the ANG-2 heavy chain variable region by one or more disulfide bonds.

In some embodiments, the light chain polypeptide is a surrogate light chain polypeptide. In some embodiments, the surrogate light chain polypeptide comprises a VpreB sequence fused to a λ5 sequence. In some embodiments, the VpreB sequence comprises an amino acid sequence comprising at least 80%, at least 85%, at least 90%, at least 95%, at least 97%, at least 98%, at least 99%, or at least 100% sequence identity to the amino acid sequence set forth in SEQ ID NO: 101. In some embodiments, the λ5 sequence comprises an amino acid sequence comprising at least 80%, at least 85%, at least 90%, at least 95%, at least 97%, at least 98%, at least 99%, or at least 100% sequence identity to the amino acid sequence set forth in SEQ ID NO: 106. In some embodiments, the surrogate light chain polypeptide comprises: a. a light chain complementarity determining region 1 (LCDR1) comprising the amino acid sequence set forth in SEQ ID NO: 103; b. a light chain complementarity determining region 2 (LCDR2) comprising the amino acid sequence set forth in SEQ ID NO: 104; and c. a light chain complementarity determining region 3 (LCDR3) comprising the amino acid sequence set forth in SEQ ID NO: 105. In some embodiments, the surrogate light chain polypeptide comprises an amino acid sequence comprising at least 80%, at least 85%, at least 90%, at least 95%, at least 97%, at least 98%, or at least 99% identity to the amino acid sequence set forth in SEQ ID NO: 102. In some embodiments, the surrogate light chain polypeptide comprises the amino acid sequence set forth in SEQ ID NO: 102. In some embodiments, the light chain polypeptide is a common light chain polypeptide.

Described herein in one aspect, is a multispecific antigen binding polypeptide comprising: an ANG-2 heavy chain variable region paired to a light chain polypeptide, wherein the ANG-2 heavy chain variable region comprises: a heavy chain complementarity determining region 1 (HCDR1) comprising the amino acid sequence set forth in any one of SEQ ID NOs: 13, 41, 44, and 47; a heavy chain complementarity determining region 2 (HCDR2) comprising the amino acid sequence set forth in any one of SEQ ID NOs: 14, 42, 45, and 48; and a heavy chain complementarity determining region 3 (HCDR3) comprising the amino acid sequence set forth in any one of SEQ ID NOs: 15, 43, 46, and 49.

In some embodiments, the ANG-2 heavy chain variable region comprises an amino acid sequence comprising at least 85%, 90%, 95%, 97%, 98%, or 99% identity to the amino acid sequence set forth in SEQ ID NO: 11. In some embodiments, the ANG-2 heavy chain variable region comprises the amino acid sequence set forth in SEQ ID NO: 11. In some embodiments, the ANG-2 binding moiety comprises an amino acid sequence comprising at least 85%, 90%, 95%, 97%, 98%, or 99% identity to the amino acid sequence set forth in SEQ ID NO: 12. In some embodiments, the ANG-2 binding moiety comprises the amino acid sequence set forth in SEQ ID NO: 12. In some embodiments, the light chain polypeptide is non-covalently associated with the ANG-2 heavy chain variable region. In some embodiments, the light chain polypeptide is covalently associated with the ANG-2 heavy chain variable region. In some embodiments, the light chain polypeptide is covalently associated with the ANG-2 heavy chain variable region by one or more disulfide bonds.

In some embodiments, the multispecific antigen binding polypeptide comprises a VEGF-A binding moiety, wherein the VEGF-A binding moiety comprises a VEGF-A heavy chain variable region paired with a light chain polypeptide. In some embodiments, the VEGF-A heavy chain variable region comprises: a. a heavy chain complementarity determining region 1 (HCDR1) comprising the amino acid sequence set forth in any one of SEQ ID NOs: 3, 31, 34, and 37; b. a heavy chain complementarity determining region 2 (HCDR2) comprising the amino acid sequence set forth in any one of SEQ ID NOs: 4, 32, 35, and 38; and c. a heavy chain complementarity determining region 3 (HCDR3) comprising the amino acid sequence set forth in any one of SEQ ID NOs: 5, 33, 36, and 39. In some embodiments, the VEGF-A heavy chain variable region comprises an amino acid sequence comprising at least 85%, 90%, 95%, 97%, 98%, or 99% identity to the amino acid sequence set forth in SEQ ID NO: 1. In some embodiments, the VEGF-A heavy chain variable region comprises the amino acid sequence set forth in SEQ ID NO: 1. In some embodiments, the VEGF-A binding moiety comprises an amino acid sequence comprising at least 85%, 90%, 95%, 97%, 98%, or 99% identity to the amino acid sequence set forth in SEQ ID NO: 2. In some embodiments, the VEGF-A binding moiety comprises the amino acid sequence set forth in SEQ ID NO: 2. In some embodiments, the light chain polypeptide is non-covalently associated with the VEGF-A heavy chain variable region. In some embodiments, the light chain polypeptide is covalently associated with the VEGF-A heavy chain variable region. In some embodiments, the light chain polypeptide is covalently associated with the VEGF-A heavy chain variable region by one or more disulfide bonds.

In some embodiments, the multispecific antigen binding polypeptide comprises a VEGF-C binding moiety, wherein the VEGF-C binding moiety comprises a VEGF-C heavy chain variable region paired with a light chain polypeptide. In some embodiments, the VEGF-C heavy chain variable region comprises: a. a heavy chain complementarity determining region 1 (HCDR1) comprising the amino acid sequence set forth in any one of SEQ ID NOs: 23, 51, 54, and 57; b. a heavy chain complementarity determining region 2 (HCDR2) comprising the amino acid sequence set forth in any one of SEQ ID NOs: 24, 52, 55, and 58; and c. a heavy chain complementarity determining region 3 (HCDR3) comprising the amino acid sequence set forth in any one of SEQ ID NOs: 25, 53, 56, and 59. In some embodiments, the VEGF-C heavy chain variable region comprises an amino acid sequence comprising at least 85%, 90%, 95%, 97%, 98%, or 99% identity to the amino acid sequence set forth in SEQ ID NO: 21. In some embodiments, the VEGF-C heavy chain variable region comprises the amino acid sequence set forth in SEQ ID NO: 21. In some embodiments, the VEGF-C binding moiety comprises an amino acid sequence comprising at least 85%, 90%, 95%, 97%, 98%, or 99% identity to the amino acid sequence set forth in SEQ ID NO: 22. In some embodiments, the VEGF-C binding moiety comprises the amino acid sequence set forth in SEQ ID NO: 22. In some embodiments, the light chain polypeptide is non-covalently associated with the VEGF-C heavy chain variable region. In some embodiments, the light chain polypeptide is covalently associated with the VEGF-C heavy chain variable region. In some embodiments, the light chain polypeptide is covalently associated with the VEGF-C heavy chain variable region by one or more disulfide bonds.

In some embodiments, the multispecific antigen binding polypeptide comprises an amino acid sequence comprising at least 85%, 90%, 95%, 97%, 98%, or 99% identity to the amino acid sequence set forth in SEQ ID NO: 201. In some embodiments, the multispecific antigen binding polypeptide comprises the amino acid sequence set forth in SEQ ID NO: 201. In some embodiments, the light chain polypeptide is a common light chain polypeptide. In some embodiments, the light chain polypeptide is a surrogate light chain polypeptide.

In some embodiments, the surrogate light chain polypeptide comprises a VpreB sequence fused to a λ5 sequence. In some embodiments, the VpreB sequence comprises an amino acid sequence comprising at least 80%, at least 85%, at least 90%, at least 95%, at least 97%, at least 98%, at least 99%, or at least 100% sequence identity to the amino acid sequence set forth in SEQ ID NO: 101. In some embodiments, the λ5 sequence comprises an amino acid sequence comprising at least 80%, at least 85%, at least 90%, at least 95%, at least 97%, at least 98%, at least 99%, or at least 100% sequence identity to the amino acid sequence set forth in SEQ ID NO: 106. In some embodiments, the surrogate light chain polypeptide comprises: a. a light chain complementarity determining region 1 (LCDR1) comprising the amino acid sequence set forth in SEQ ID NO: 103; b. a light chain complementarity determining region 2 (LCDR2) comprising the amino acid sequence set forth in SEQ ID NO: 104; and c. a light chain complementarity determining region 3 (LCDR3) comprising the amino acid sequence set forth in SEQ ID NO: 105. In some embodiments, the surrogate light chain polypeptide comprises an amino acid sequence comprising at least 80%, at least 85%, at least 90%, at least 95%, at least 97%, at least 98%, or at least 99% identity to the amino acid sequence set forth in SEQ ID NO: 102. In some embodiments, the surrogate light chain polypeptide comprises the amino acid sequence set forth in SEQ ID NO: 102.

Described herein in another aspect is multispecific antigen binding polypeptides comprising: (a) a VEGF-C binding moiety, wherein the VEGF-C binding moiety comprises a VEGF-C heavy chain variable region paired to a first surrogate light chain polypeptide; (b) a VEGF-A binding moiety, wherein the VEGF-A binding moiety comprises a VEGF-A heavy chain variable region paired to a second surrogate light chain polypeptide; and (c) an ANG-2 binding moiety, wherein the ANG-2 binding moiety comprises a ANG-2 heavy chain variable region paired to a third surrogate light chain polypeptide; wherein the VEGF-C heavy chain variable region comprises: a heavy chain complementarity determining region 1 (HCDR1) comprising the amino acid sequence set forth in any one of SEQ ID NOs: 23, 51, 54, and 57; a heavy chain complementarity determining region 2 (HCDR2) comprising the amino acid sequence set forth in any one of SEQ ID NOs: 24, 52, 55, and 58; and a heavy chain complementarity determining region 3 (HCDR3) comprising the amino acid sequence set forth in any one of SEQ ID NOs: 25, 53, 56, and 59; wherein the VEGF-A heavy chain variable region comprises: (a) a heavy chain complementarity determining region 1 (HCDR1) comprising the amino acid sequence set forth in any one of SEQ ID NOs: 3, 31, 34, and 37; a heavy chain complementarity determining region 2 (HCDR2) comprising the amino acid sequence set forth in any one of SEQ ID NOs: 4, 32, 35, and 38; and a heavy chain complementarity determining region 3 (HCDR3) comprising the amino acid sequence set forth in any one of SEQ ID NOs: 5, 33, 36, and 39; wherein the ANG-2 heavy chain variable region comprises: a heavy chain complementarity determining region 1 (HCDR1) comprising the amino acid sequence set forth in any one of SEQ ID NOs: 13, 41, 44, and 47; a heavy chain complementarity determining region 2 (HCDR2) comprising the amino acid sequence set forth in any one of SEQ ID NOs: 14, 42, 45, and 48; and a heavy chain complementarity determining region 3 (HCDR3) comprising the amino acid sequence set forth in any one of SEQ ID NOs: 15, 43, 46, and 49; and wherein the first, second, and third surrogate light chain polypeptide comprises: a light chain complementarity determining region 1 (LCDR1) comprising the amino acid sequence set forth in SEQ ID NO: 103; a light chain complementarity determining region 2 (LCDR2) comprising the amino acid sequence set forth in SEQ ID NO: 104; and a light chain complementarity determining region 3 (LCDR3) comprising the amino acid sequence set forth in SEQ ID NO: 105.

In some embodiments, the ANG-2 binding moiety, the VEGF-A binding moiety, and/or the VEGF-C binding moiety comprises a heavy chain first constant region (CH1). In some embodiments, the heavy chain first constant region comprises an amino acid sequence with at least 80%, at least 90%, at least 95%, at least 97%, at least 98%, at least 99%, or at least 100% identity to the amino acid sequence set forth in SEQ ID NO: 141. In some embodiments, the multispecific antigen binding polypeptide comprises an amino acid sequence comprising at least 85%, at least 90%, at least 95%, at least 97%, at least 98%, or at least 99% identity to the amino acid sequence set forth in SEQ ID NO: 201. In some embodiments, the multispecific antigen binding polypeptide comprises the amino acid sequence set forth in SEQ ID NO: 201. In some embodiments, the first, second, and third surrogate light chain polypeptide comprises an amino acid sequence comprising at least 85%, at least 90%, at least 95%, at least 97%, at least 98%, or at least 99% identity to the amino acid sequence set forth in SEQ ID NO: 102. In some embodiments, the first, second, and third surrogate light chain polypeptide comprises the amino acid sequence set forth in SEQ ID NO: 102.

In some embodiments, the multispecific antigen binding polypeptide binds to ANG-2 or a fragment thereof with an EC50 of about 99 picomolar or less. In some embodiments, the multispecific antigen binding polypeptide binds to VEGF-A or a fragment thereof with an EC50 of about 19 picomolar or less. In some embodiments, the multispecific antigen binding polypeptide binds to VEGF-C or a fragment thereof with an EC50 of about 63 picomolar or less. In some embodiments, the multispecific antigen binding polypeptide inhibits the interaction of ANG-2 and the ANG-2 receptor with an IC50 of about 13.6 nanomolar or less. In some embodiments, the multispecific antigen binding polypeptide inhibits the interaction of VEGF-C and the VEGF-C receptor with an IC50 of about 9.8 nanomolar or less. In some embodiments, the multispecific antigen binding polypeptide inhibits the interaction of VEGF-A and the VEGF-A receptor 2 with an IC50 of about 41 nanomolar or less. In some embodiments, the pre-existing anti-drug antibodies (ADAs) titer in the pooled human serum against the multispecific binding polypeptide is less than 1:2700.

Described herein are nucleic acids or plurality of nucleic acids encoding the ANG-2 binding antibody or antigen binding fragment described herein or the multispecific antigen binding polypeptides described herein.

Described herein are expression vector comprising the nucleic acids described herein. In some embodiments, the expression vector is RNA. In some embodiments, the expression vector is plasmid or linearized DNA. In some embodiments, the expression vector is a viral vector. In some embodiments, the viral vector is an adenovirus. In some embodiments, the viral vector is an adeno-associated virus. In some embodiments, the viral vector is a lentivirus virus. Described herein are cells comprising the expression vector described herein. In some embodiments, the cell is eukaryotic. In some embodiments, the cell is Chinese Hamster Ovary (CHO) cell.

Described herein are pharmaceutical compositions comprising the ANG-2 binding antibody or antigen binding fragment described herein, the multispecific antigen binding polypeptides described herein, or the nucleic acid or plurality of nucleic acids described herein, and a pharmaceutically acceptable carrier, excipient, or diluent. In some embodiments, the pharmaceutical composition is formulated for intravenous administration. In some embodiments, the pharmaceutical composition is formulated for intravitreal administration.

Described herein are methods of treating an ocular disease in an individual comprising administering to the individual the ANG-2 binding antibody or antigen binding fragment described herein or the multispecific antigen binding polypeptides described herein, thereby treating the ocular disease. In some embodiments, the ocular disease comprises macular degeneration, diabetic macular edema, or retinal vein occlusion. Also described herein are methods of making the ANG-2 binding antibody or antigen binding fragment described herein or the multispecific antigen binding polypeptides described herein, comprising culturing a cell comprising the nucleic acids described herein under conditions sufficient for expression of the nucleic acid and isolating or purifying the ANG-2 binding antibody or antigen binding fragment thereof or the multispecific antigen binding polypeptide or the antigen binding polypeptide from the cell or a supernatant there of.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1A is an exemplary trispecific antigen binding polypeptide comprising three binding moieties that bind to three different targets. Each of the three binding moieties comprises a variable heavy chain region (V_{H}) linked to a heavy chain first constant region (CH1). Each of the three binding moieties also comprises a VpreB domain linked to a λ5 domain.

FIG. 1B is an exemplary trispecific antigen binding polypeptide comprising three binding moieties that bind to VEGF-A, ANG-2, and VEGF-C. Each of the three binding moieties comprises a variable heavy chain region (V_{H}) linked to a heavy chain first constant region (CH1). Each of the three binding moieties also comprises a VpreB domain linked to a λ5 domain.

FIG. 2 is an exemplary trispecific antigen binding polypeptide comprising three binding moieties that bind to three different targets. One of the binding moieties that binds to target 1 comprises a crossed domain. Each of the three binding moieties comprises a variable heavy chain region (V_{H}) and a heavy chain first constant region (CH1). Each of the three binding moieties also comprises a VpreB domain and a λ5 domain or a variable light chain region (V_{L}) and a constant light chain region (CL).

FIG. 3 is an exemplary trispecific antigen binding polypeptide comprising three binding moieties that bind to three different targets. Two of the binding moieties comprise a crossed domain. Each of the three binding moieties comprises a variable heavy chain region (V_{H}) and a heavy chain first constant region (CH1). Each of the three binding moieties also comprises a VpreB domain and a λ5 domain or a variable light chain region (V_{L}) linked to a constant light chain region (CL).

FIG. 4 is an exemplary trispecific antigen binding polypeptide comprising three binding moieties that bind to three different targets. Each of the three binding moieties comprises a variable heavy chain region (V_{H}) linked to a heavy chain first constant region (CH1). Each of the three binding moieties also comprises a variable light chain region (V_{L}) linked to a constant light chain region (CL).

FIGS. 5A-5B illustrate SDS-PAGE and SE-HPLC analysis of RP-043a trispecific antigen binding polypeptide that binds to VEGF-A, ANG-2 and VEGF-C. FIG. 5A illustrates SDS-PAGE results for reduced and non-reduced samples. FIG. 5B illustrates an SE-HPLC graph of the trispecific antigen binding polypeptide.

FIGS. 6A-6C illustrate trispecific RP-043's binding affinity to VEGF-A, ANG-2, and VEGF-C in comparison with Faricimab, captured by the biotinylated anti-IgG-CH1 antibody. FIG. 6A illustrates ELISA-binding results for RP-043's binding to VEGF-A. FIG. 6B illustrates ELISA-binding results for RP-043's binding to VEGF-C. FIG. 6C illustrates ELISA-binding results for RP-043's binding to ANG-2.

FIGS. 7A-7B illustrate the binding affinity of RP-043 to ANG-2 and ANG-1 after being captured by the biotinylated anti-VpreB antibody. FIG. 7A illustrates ELISA-binding results for RP-043's binding to ANG-2. FIG. 7B illustrates ELISA-binding results for RP-043's binding to ANG-1.

FIGS. 8A-8B illustrate the binding affinity of RP-043 and faricimab after being captured by the biotinylated VEGF-A. FIG. 8A illustrates ELISA-binding results for faricimab and RP-043's binding to ANG-2. FIG. 8B illustrates ELISA-binding results for faricimab and RP-043's binding to ANG-1.

FIGS. 9A-9B illustrate RP-043 and faricimab′ inhibition of the interaction between VEGF and its receptor VEGFR2. FIG. 9A shows RP-043 and faricimab′ inhibition of the interaction between VEGF-A and VEGFR2. FIG. 9B shows RP-043 and faricimab′ inhibition of the interaction between VEGF-C and VEGFR2.

FIGS. 10A-10B illustrate RP-043 and faricimab′ inhibition of the interaction between ANG-2 and ANG-1 and their receptor Tie2. FIG. 10A shows RP-043 and faricimab′ inhibition of the interaction between ANG-2 and Tie2. FIG. 10B shows RP-043 and faricimab′ inhibition of the interaction between ANG-1 and Tie2.

FIG. 11 illustrates RP-043's inhibition of VEGF-A and VEGF-C stimulated pathways in bioassay.

FIG. 12 illustrates RP-043 and faricimab's inhibition of ANG-2 stimulated pathway in bioassay.

FIG. 13 illustrates RP-043′ inhibition of the interaction between cynomolgus monkey Tie2 and ANG-1.

FIGS. 14A-14C illustrate the binding affinity of RP-043 to VEGF-A, ANG-2, and VEGF-C of different species. FIG. 14A illustrates the binding affinity of RP-043 to human, rabbit, and Rat VEGF-A. FIG. 14B illustrates the binding affinity of RP-043 to human, cynomolgus monkey, rabbit, and Rat ANG-2. FIG. 14C illustrates the binding affinity of RP-043 to human, cynomolgus monkey, rabbit, and Rat VEGF-C.

FIGS. 15A-15D illustrate RP-043 binding to VEGF-A, ANG-2, VEGF-C, and ANG-1 assessed by Biolayer Interferometry. FIG. 15A illustrates the binding affinity of RP-043 to VEGF-A. FIG. 15B illustrates the binding affinity of RP-043 to ANG-2. FIG. 15C illustrates the binding affinity of RP-043 to VEGF-C. FIG. 15D illustrates the binding affinity of RP-043 to ANG-1.

FIGS. 16A-16D illustrate RP-019 binding to VEGF-A, ANG-2, VEGF-C, and ANG-1 assessed by Biolayer Interferometry. FIG. 16A illustrates the binding affinity of RP-019 to VEGF-A. FIG. 16B illustrates the binding affinity of RP-019 to ANG-2. FIG. 16C illustrates the binding affinity of RP-019 to VEGF-C. FIG. 16D illustrates the binding affinity of RP-019 to ANG-1.

FIG. 17 shows changes in lesion grade after treatment with RP-019 in the nonhuman primate laser-induced choroidal neovascularization (LCNV) model. Vehicle group received vehicle control. Low dose group received 0.27 mg of RP-019. High dose group received 1.09 mg of RP-019. LCNV lesions were induced in both eyes of the animals. Each lesion was graded and assigned a score of 1 to 4 on a clinical severity scale.

FIG. 18 shows the corrected total lesion fluorescence on day 8, after being treated with the vehicle, 0.066 mg/eye aflibercept, 0.1 mg/eye faricimab, or 0.1 mg/eye RP-043 in a rat model.

FIG. 19 illustrates retinal vascular images 8 days after treatment with vehicles, aflibercept, faricimab, or RP-043 in a rat model.

FIGS. 20A-20D illustrate efficacy of RP-043 in a laser-induced choroidal neovascularization (CNV) model in nonhuman primate. FIG. 20A summarizes the study protocol design. FIG. 20B shows changes in percentage of grade 3 and grade 4 lesions after treatment in different groups treated with RP-043, faricimab, or control vehicle. Vehicle group received vehicle control. Faricimab group received 1 mg of faricimab in each eye. RP-043 group received 1 mg of RP-043 in each eye. FIG. 20C shows the efficacy in percentage reduction of grade 3 and grade 4 lesions after treatment. FIG. 20D shows the pre-dose and post-dose OCT images of the eyes in different groups treated with RP-043, faricimab, or vehicle control.

FIG. 21 shows the concentrations of RP-043 in the terminal eye tissues on Day 14 post the 3^{rd} dosing.

### DETAILED DESCRIPTION

Described herein in one aspect, is Angiopoietin-2 (ANG-2) binding antibodies or antigen binding fragment thereof comprising: an ANG-2 heavy chain variable region paired to a light chain polypeptide, wherein the ANG-2 heavy chain variable region comprises: a heavy chain complementarity determining region 1 (HCDR1) comprising the amino acid sequence set forth in any one of SEQ ID NOs: 13, 41, 44, and 47; a heavy chain complementarity determining region 2 (HCDR2) comprising the amino acid sequence set forth in any one of SEQ ID NOs: 14, 42, 45, and 48; and a heavy chain complementarity determining region 3 (HCDR3) comprising the amino acid sequence set forth in any one of SEQ ID NOs: 15, 43, 46, and 49.

Described herein in one aspect, is a multispecific antigen binding polypeptide comprising: an ANG-2 heavy chain variable region paired to a light chain polypeptide, wherein the ANG-2 heavy chain variable region comprises: a heavy chain complementarity determining region 1 (HCDR1) comprising the amino acid sequence set forth in any one of SEQ ID NOs: 13, 41, 44, and 47; a heavy chain complementarity determining region 2 (HCDR2) comprising the amino acid sequence set forth in any one of SEQ ID NOs: 14, 42, 45, and 48; and a heavy chain complementarity determining region 3 (HCDR3) comprising the amino acid sequence set forth in any one of SEQ ID NOs: 15, 43, 46, and 49.

Described herein in another aspect is multispecific antigen binding polypeptides comprising: (a) a VEGF-C binding moiety, wherein the VEGF-C binding moiety comprises a VEGF-C heavy chain variable region paired to a first surrogate light chain polypeptide; (b) a VEGF-A binding moiety, wherein the VEGF-A binding moiety comprises a VEGF-A heavy chain variable region paired to a second surrogate light chain polypeptide; and (c) an ANG-2 binding moiety, wherein the ANG-2 binding moiety comprises a ANG-2 heavy chain variable region paired to a third surrogate light chain polypeptide. In some embodiments, the VEGF-C heavy chain variable region comprises: a heavy chain complementarity determining region 1 (HCDR1) comprising the amino acid sequence set forth in any one of SEQ ID NOs: 23, 51, 54, and 57; a heavy chain complementarity determining region 2 (HCDR2) comprising the amino acid sequence set forth in any one of SEQ ID NOs: 24, 52, 55, and 58; and a heavy chain complementarity determining region 3 (HCDR3) comprising the amino acid sequence set forth in any one of SEQ ID NOs: 25, 53, 56, and 59. In some embodiments, the VEGF-A heavy chain variable region comprises: (a) a heavy chain complementarity determining region 1 (HCDR1) comprising the amino acid sequence set forth in any one of SEQ ID NOs: 3, 31, 34, and 37; a heavy chain complementarity determining region 2 (HCDR2) comprising the amino acid sequence set forth in any one of SEQ ID NOs: 4, 32, 35, and 38; and a heavy chain complementarity determining region 3 (HCDR3) comprising the amino acid sequence set forth in any one of SEQ ID NOs: 5, 33, 36, and 39. In some embodiments, the ANG-2 heavy chain variable region comprises: a heavy chain complementarity determining region 1 (HCDR1) comprising the amino acid sequence set forth in any one of SEQ ID NOs: 13, 41, 44, and 47; a heavy chain complementarity determining region 2 (HCDR2) comprising the amino acid sequence set forth in any one of SEQ ID NOs: 14, 42, 45, and 48; and a heavy chain complementarity determining region 3 (HCDR3) comprising the amino acid sequence set forth in any one of SEQ ID NOs: 15, 43, 46, and 49. In some embodiments, the first, second, and third surrogate light chain polypeptide comprises: a light chain complementarity determining region 1 (LCDR1) comprising the amino acid sequence set forth in SEQ ID NO: 103; a light chain complementarity determining region 2 (LCDR2) comprising the amino acid sequence set forth in SEQ ID NO: 104; and a light chain complementarity determining region 3 (LCDR3) comprising the amino acid sequence set forth in SEQ ID NO: 105.

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein “vascular endothelial growth factor A” or “VEGF-A” refers to the polypeptide encoded by the VEGFA gene, which amino acid sequence is available at www.uniprot.org/uniprot/P15692. Such sequence includes post-translational modifications, naturally occurring amino acid variants, and naturally occurring splice variants that do not affect the binding of the antigen binding polypeptides and bispecific antigen binding polypeptides described herein.

As used herein “Angiopoietin 2” or “ANG-2” refers to the polypeptide encoded by the ANGPT2 gene, which amino acid sequence is available at www.uniprot.org/uniprot/O15123. Such sequence includes post-translational modifications, naturally occurring amino acid variants, and naturally occurring splice variants that do not affect the binding of the antigen binding polypeptides and bispecific antigen binding polypeptides described herein.

As used herein “vascular endothelial growth factor C” or “VEGF-C” refers to the polypeptide encoded by the VEGFC gene, which amino acid sequence is available at www.uniprot.org/uniprot/P49767. Such sequence includes post-translational modifications, naturally occurring amino acid variants, and naturally occurring splice variants that do not affect the binding of the antigen binding polypeptides and bispecific antigen binding polypeptides described herein.

As used herein a “surrogate light chain” refers to a light chain that is expressed during early B cell development that can associate promiscuously with two or more different expressed heavy chains. A specific surrogate light chain can be formed by fusion of a VpreB polypeptide and λ5 polypeptide, and can promiscuously associate with two or more structurally distinct heavy chains. Such surrogate light chains are described in U.S. Pat. Nos. 8,114,967; 10,214,580; and 8,969,082, each of which are incorporated herein in their entirety.

As used herein a “surrogate light chain variable region” refers to a light chain variable region comprises a VpreB polypeptide. The VpreB polypeptide can be a VpreB1, VpreB2, or VpreB3 polypeptide. In some embodiments, the VpreB sequence comprises an amino acid sequence comprising at least 90%, at least 95%, at least 97%, at least 98%, at least 99%, or at least 100% sequence identity to the amino acid sequences set forth in SEQ ID NO: 101. Such surrogate light chain variable regions are described in U.S. Pat. Nos. 8,114,967; 10,214,580; and 8,969,082, each of which are incorporated herein in their entirety.

As used herein a “common light chain” or “universal light chain” refers to a single light chain that can promiscuously associate with two or more structurally distinct heavy chains.

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, monkey, rabbit, dog, cat, horse, cow, sheep, pig, goat, llama, alpaca, or yak. In certain embodiments, the individual is a human.

Among the provided antigen binding polypeptides are monoclonal antibodies, multispecific antibodies (for example, bispecific antibodies, trispecific antibodies, and polyreactive antibodies), and antibody fragments. The antibodies include antibody-conjugates and molecules comprising the antibodies, such as chimeric molecules. Thus, an antibody includes, but is not limited to, full-length and native antibodies, as well as fragments and portion thereof retaining the binding specificities thereof, such as any specific binding portion thereof including those having any number of, immunoglobulin classes and/or isotypes (e.g., IgG1, IgG2, IgG3, IgG4, IgM, IgA, IgD, IgE and IgM); and biologically relevant (antigen-binding) fragments or specific binding portions thereof, including but not limited to Fab, F(ab′)₂, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. The monoclonal antibody can comprise a human IgG1 constant region. The monoclonal antibody can comprise a human IgG4 constant region.

The term “antibody” herein is used in the broadest sense and includes monoclonal antibodies, bispecific antibodies, multispecific antibodies, and includes intact antibodies and functional (antigen-binding) antibody fragments thereof, including fragment antigen binding (Fab) fragments, F(ab′)₂ fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region.

The term “antigen binding polypeptide” or “antigen binding moiety” herein is used in the broadest sense and includes polypeptides that bind to an antigen. The term may include monoclonal antibodies, bispecific antibodies, multispecific antibodies, and includes intact antibodies and functional (antigen-binding) antibody fragments thereof, including fragment antigen binding (Fab) fragments, F(ab′)₂ fragments, Fab′ fragments, Fv fragments, recombinant IgG (rIgG) fragments, single chain antibody fragments, including single chain variable fragments (sFv or scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, surrobodies, fully human antibodies, humanized antibodies, and heteroconjugate antibodies, multispecific, e.g., bispecific, antibodies, diabodies, triabodies, and tandem di-scFv, tandem tri-scFv. Unless otherwise stated, the term “antigen binding polypeptide” should be understood to encompass functional antigen binding polypeptide and fragments thereof. The term also encompasses intact or full-length antibodies, including antibodies of any class or sub-class, including IgG and sub-classes thereof, IgM, IgE, IgA, and IgD.

As used herein the terms “heavy chain” or “light chain” relate to polypeptides derived from or similar to heavy chains or light chains of antibodies or antibody like molecules. The light chains and heavy chains of such antibodies or antibody like molecules may consist of several domains. In a whole antibody, each heavy chain comprises a heavy chain variable region (abbreviated herein as VH) and a heavy chain constant region. The heavy chain constant region comprises the heavy chain constant domains CH1, hinge, CH2 and CH3 (antibody classes IgA, IgD, and IgG) and optionally the heavy chain constant domain CH4 (antibody classes IgE and IgM). Each light chain comprises a light chain variable domain (abbreviated herein as VL) and a light chain constant domain CL.

Herein a molecule, peptide, polypeptide, antibody, or antibody fragment can be referred to as “bispecific” or “dual-specific” including grammatical equivalents. A bispecific molecule possesses the ability to specifically bind to at least two structurally distinct targets. The specific binding may be the result of two distinct binding moieties that are structurally distinct at the molecular level, including but not limited to distinct non-identical amino acid sequences; or a single binding moiety that is able to specifically bind to two structurally distinct targets with high affinity (e.g., with a KD less than about 1×10⁻⁶). A molecule, peptide, polypeptide, antibody, or antibody fragment referred to as “multi-specific” refers to a molecule that possesses the ability to specifically bind to at least three structurally distinct targets. A “bispecific antibody” including grammatical equivalents refers to a bispecific molecule that preserves at least one fragment of an antibody able to specifically bind a target, for example, a variable region, heavy or light chain, or one or more complementarity determining regions from an antibody molecule.

Herein a molecule, peptide, polypeptide, antibody, or antibody fragment can be referred to as “trispecific” including grammatical equivalents. A trispecific molecule possesses the ability to specifically bind to at least three structurally distinct targets. The specific binding may be the result of three distinct binding moieties that are structurally distinct at the molecular level, including but not limited to distinct non-identical amino acid sequences; or a single binding moiety that is able to specifically bind to three structurally distinct targets with high affinity (e.g., with a KD less than about 1×10⁻⁶). Herein a molecule, peptide, polypeptide, antibody, or antibody fragment can be referred to as “tetraspecific” including grammatical equivalents. A tetraspecific molecule possesses the ability to specifically bind to at least four structurally distinct targets. The specific binding may be the result of four distinct binding moieties that are structurally distinct at the molecular level, including but not limited to distinct non-identical amino acid sequences; or a single binding moiety that is able to specifically bind to four structurally distinct targets with high affinity (e.g., with a KD less than about 1×10⁻⁶). A molecule, peptide, polypeptide, antibody, or antibody fragment referred to as “multi-specific” refers to a molecule that possesses the ability to specifically bind to at least three structurally distinct targets. A “multi-specific antibody” including grammatical equivalents refers to a multi-specific molecule that preserves at least one fragment of an antibody able to specifically bind with a target, for example, a variable region, heavy or light chain, or complementarity determining region from an antibody molecule.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Pluckthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments, the CDRs of the antigen binding polypeptides described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single V_{H} or V_{L} domain may be sufficient to confer antigen-binding specificity. Furthermore, antigen binding polypeptides that bind a particular antigen may be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

The terms “crossed domain” or “CrossMab” refers to the light chain constant and heavy chain constant domains of an antibody or an antigen binding moiety being crossed over. A crossed domain may comprise one or more amino acid substitutions to a light chain constant domain corresponding to one or more amino acids from an immunoglobulin CH1 domain and vice versa. A crossed domain may comprise one or more amino acid substitutions to a surrogate light chain constant domain corresponding to one or more amino acids from an immunoglobulin CH1 domain and vice versa. The crossed domain enforces correct light chain association in the multispecific antigen binding polypeptides (See e.g., Surowka et al., MABS. 13(1): E1967714 (2021)).

Specific binding or binding of antigen binding polypeptide molecules described herein refers to binding mediated by one or more CDR portions of the antigen binding polypeptide. Not all CDRs may be required for specific binding. Specific binding can be demonstrated for example by an ELISA against a specific recited target or antigen that shows significant increase in binding compared to an isotype control antigen binding polypeptide.

As described herein an “epitope” refers to the binding determinant of an antigen binding polypeptide or fragment described herein minimally necessary for specific binding of the antigen binding polypeptide or fragment thereof to a target antigen. When the target antigen is a polypeptide the epitope will be a continuous or discontinuous epitope. A continuous epitope is formed by one region of the target antigen, while a discontinuous epitope may be formed from two or more separate regions. A discontinuous epitope, for example, may form when a target antigen adopts a tertiary structure that brings two amino acid sequences together and forms a three-dimensional structure bound by the antigen binding polypeptide. When the target antigen is a polypeptide the epitope will generally be a plurality of amino acids linked into a polypeptide chain. A continuous epitope may comprise 5, 6, 7, 8, 9, 10, 11, 12, 13, 14, 15, 16, 17, 18, 19, or 20 contiguous amino acids. While an epitope may comprise a contiguous polymer of amino acids, not every amino acid of the polymer may be contacted by an amino acid residue of the antigen binding polypeptide. Such non-contacted amino acids will still comprise part of the epitope as they may be important for the structure and linkage of the contacted amino acids. The skilled artisan may determine if any given antigen binding polypeptide binds an epitope of a reference antigen binding polypeptide, for example, by cross-blocking experiments with a reference antigen binding polypeptide. In certain embodiments, described herein, are antigen binding polypeptides that bind the same epitope of the described antigen binding polypeptides. In certain embodiments, described herein, are antigen binding polypeptides that are competitively blocked by the described antigen binding polypeptides. In certain embodiments, described herein, are antigen binding polypeptides that compete for binding with the described antigen binding polypeptides.

Among the provided antigen binding polypeptides are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv or sFv); and multispecific antigen binding polypeptides formed from antibody fragments. In particular embodiments, the antigen binding polypeptides are single-chain antibody fragments comprising a variable heavy chain region and/or a variable light chain region, such as scFvs.

Antibody fragments can be made by various techniques, including but not limited to in vitro translation, proteolytic digestion of an intact antibody as well as production by recombinant host cells. In some embodiments, the antigen binding polypeptides are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antigen binding polypeptides are human antibodies. A “human antibody” is an antibody with an amino acid sequence corresponding to that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

Human antigen binding polypeptides may be prepared by administering an immunogen to a transgenic animal that has been modified to produce intact human antigen binding polypeptides or intact antigen binding polypeptides with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antigen binding polypeptides also may be derived from human antibody libraries, including phage display and cell-free libraries, containing antibody-encoding sequences derived from a human repertoire.

The terms “polypeptide” and “protein” are used interchangeably to refer to a polymer of amino acid residues, and are not limited to a minimum length. Polypeptides, including the provided antigen binding polypeptides and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification. In some embodiments, amino acid sequence variants of the antigen binding polypeptides provided herein are contemplated. A variant typically differs from a polypeptide specifically disclosed herein in one or more substitutions, deletions, additions and/or insertions. Such variants can be naturally occurring or can be synthetically generated, for example, by modifying one or more of the above polypeptide sequences of the invention and evaluating one or more biological activities of the polypeptide as described herein and/or using any of a number of known techniques. For example, it may be desirable to improve the binding affinity and/or other biological properties of the antibody Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. References to a polypeptide described herein may refer a single polypeptide chain or a heterodimer or heteromultimer of multiple polypeptide chains thar are associated either covalently (e.g., by disulfide bonds) or non-covalently.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Amino acid sequence insertions and deletions include amino- and/or carboxyl-terminal fusions ranging in length from one residue to polypeptides containing a hundred or more residues, as well as intrasequence insertions and deletions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody. Examples of intrasequence insertion variants of the antibody molecules include an insertion of 3 amino acids in the light chain. Examples of terminal deletions include an antibody with a deletion of 7 or less amino acids at an end of the light chain.

In some embodiments, an antigen binding polypeptide provided herein has a dissociation constant (K_{D}) of about 1 μM, 100 nM, 50 nM, 40 nM, 30 nM, 20 nM, 10 nM, 5 nM, 2 nM, 1 nM, 0.5 nM, 0.1 nM, 0.05 nM, 0.01 nM or less (e.g., 10⁻⁸ M or less, e.g., from 10⁻⁸ M to 10⁻¹³ M, e.g., from 10⁻⁹ M to 10⁻¹³ M) for the antigen binding polypeptide target. In some embodiments, an antigen binding polypeptide provided herein has a dissociation constant (K_{D}) of about 100 nM, 50 nM, 40 nM, 30 nM, 20 nM, 10 nM, 5 nM, 2 nM, 1 nM, 0.5 nM, 0.1 nM, 0.05 nM, 0.01 nM, or 0.001 nM or greater (e.g., 10⁻⁸ M or less, e.g., from 10⁻⁸ M to 10⁻¹³ M, e.g., from 10⁻⁹ M to 10⁻¹³ M) for the antigen binding polypeptide target. The antigen binding polypeptide target can be an anti-VEGF or anti-ANG-2 antigen binding polypeptide. K_{D} can be measured by any suitable assay. In certain embodiments, KD can be measured using surface plasmon resonance assays (e.g., using a BIACORE®-2000, a BIACORE®-3000) or Bio-Layer Interferometry (BLI) (e.g. using Octet R8).

In certain embodiments, the antigen binding polypeptides disclosed herein bind to VEGF-A. VEGF-A may have several different splice sites thus may have various isoforms depending on splice sites. Accordingly, in certain embodiments, the antigen binding polypeptides disclosed herein bind to VEGF165. In certain embodiments, the antigen binding polypeptides disclosed herein bind to VEGF121. In certain embodiments, the antigen binding polypeptides' binding affinity to VEGF121 is predicative of the antigen binding polypeptides' efficacy in treating a disease of an eye. In certain embodiments, the antigen binding polypeptides disclosed herein bind to a peptide comprising a platelet-derived growth factor (PDGF) domain of VEGF-A. In some embodiments, the PDGF domain of VEGF-A comprises a fragment of VEGF-A ranging from about 40^{th}, 41^{st}, 42^{nd}, 43^{rd}, 44^{th}, 45^{th}, 46^{th}, 46^{th}, 47^{th}, 48^{th}, 49^{th}, or 50^{th} to about 130^{th}, 131^{st}, 132^{nd} 133^{rd}, 134^{th}, 135^{th}, 136^{th}, 137^{th}, 138^{th}, 139^{th}, or 140^{th} amino acids. In certain embodiments, the antigen binding polypeptides disclosed herein bind to a peptide comprising a VEGF-C heparin domain of VEGF-A. In some embodiments, the VEGF-C heparin domain of VEGF-A comprises a fragment of VEGF-A ranging from about 140^{th}, 141^{st}, 142^{nd}, 143^{rd}, 144^{th}, 145^{th}, 146^{th}, 147^{th}, 148^{th}, 149^{th}, or 150^{th} to about 190^{th}, 191^{st}, 192^{nd}, 193^{rd}, 194^{th}, 195^{th}, 196^{th}, 197^{th}, 198^{th}, 199^{th}, 120^{th} amino acids. In certain embodiments, the antigen binding polypeptides disclosed herein bind to VEGF-A from a species of human or a non-human (e.g., rabbits, canines (dogs), or porcine (pigs)).

In certain embodiments, the antigen binding polypeptides are anti-Human VEGF-A antigen binding polypeptides which bind to human VEGF-A at a K_{D} or EC₅₀ of 100, 75, 60, 50, 40, 30, or 25 picomolar or less. In certain embodiments, the antigen binding polypeptides are anti-Human VEGF-A antigen binding polypeptides which bind to human VEGF at a K_{D} or EC₅₀ of 100, 75, 60, 50, 40, 30, or 25 picomolar or less. In certain embodiments, the antigen binding polypeptides are anti-Human VEGF-A antigen binding polypeptides which bind to human VEGF at a K_{D} or EC₅₀ of 1, 2, 3, 4, 5, 6, 7, 8, 9, or 10 picomolar or more. In certain embodiments, the antigen binding polypeptides disclosed herein bind to VEGF-A or a fragment thereof with a similar EC₅₀ as the one between Faricimab and VEGF-A or a fragment thereof. In certain embodiments, the antigen binding polypeptides disclosed herein bind to VEGF-A or a fragment thereof with an EC₅₀ 10, 20, 30, 40, 50, 60, 70, 80, 90, 100, 200, 300, 400, 500 pM less than the one between Faricimab and VEGF-A or a fragment thereof.

In certain embodiments, the antigen binding polypeptides are anti-Human VEGF-A antigen binding polypeptides which inhibits an interaction of VEGF-A or a fragment thereof and a VEGF receptor. In certain embodiments, the antigen binding polypeptides are anti-Human VEGF-A antigen binding polypeptides which inhibits an interaction of VEGF-A or a fragment thereof and a VEGF receptor at an IC₅₀ of 5 nM, 4 nM, 3 nM, 2 nM, 1 nM, 900 pM, 800 pM, 700 pM, 600 pM or less. In certain embodiments, the antigen binding polypeptides are anti-Human VEGF-A antigen binding polypeptides which inhibits an interaction of VEGF-A or a fragment thereof and a VEGF receptor at an IC₅₀ of 1, 2, 3, 4, 5, 6, 7, 8, 9, or 10 picomolar or more. In certain embodiments, the antigen binding polypeptides disclosed herein inhibit the interaction of VEGF_A or a fragment thereof and the VEGF receptor at least 1-fold, 2-fold, 3-fold, 4-fold, 5-fold, 6-fold, 7-fold, 8-fold, 9-fold, or 10-fold stronger than Faricimab inhibits the interaction. In certain embodiments, the VEGF receptor is VEGF receptor 2/kinase insert domain receptor (KDR).

In certain embodiments, the antigen binding polypeptides disclosed herein bind to VEGF-B significantly less than to VEGF-A. In certain embodiments, the antigen binding polypeptides disclosed herein bind to VEGF-C significantly less than to VEGF-A. In certain embodiments, the antigen binding polypeptides disclosed herein bind to VEGF-D significantly less than to VEGF-A. In certain embodiments, the antigen binding polypeptides disclosed herein bind to PIGF-1 significantly less than to VEGF-A. In certain embodiments, the antigen binding polypeptides disclosed herein bind to PIGF-3 significantly less than to VEGF-A.

In certain embodiments, the antigen binding polypeptides are anti-Human ANG-2 antigen binding polypeptides which bind to human ANG-2 at a K_{D} or EC₅₀ of 100, 75, 60, 55, 50, 40, 30, or 25 picomolar or less. In certain embodiments, the antigen binding polypeptides are anti-Human ANG-2 antigen binding polypeptides which bind to human ANG-2 at a K_{D} or EC₅₀ of 1, 2, 3, 4, 5, 6, 7, 8, 9, or 10 picomolar or more. In certain embodiments, the antigen binding polypeptides bind to ANG-2 at least 10-fold, 20-fold, 30-fold, 40-fold, 50-fold, 60-fold, or 70-fold stronger than Faricimab binds to ANG-2.

In certain embodiments, the antigen binding polypeptides are anti-Human ANG-2 antigen binding polypeptides which inhibits an interaction of ANG-2 and an ANG-2 receptor. In certain embodiments, the antigen binding polypeptides are anti-Human VEGF-A antigen binding polypeptides which inhibits an interaction of ANG-2 and an ANG-2 receptor at an IC₅₀ of 5 nM, 4 nM, 3 nM, 2 nM, 1 nM, 900 pM, 800 pM, 700 pM, 600 pM or less. In certain embodiments, the antigen binding polypeptides are anti-Human VEGF-A antigen binding polypeptides which inhibits an interaction of ANG-2 and an ANG-2 receptor at an IC₅₀ of 1, 2, 3, 4, 5, 6, 7, 8, 9, or 10 picomolar or more. In some embodiments, the antigen binding polypeptides disclosed herein inhibit the interaction of ANG-2 and the ANG-2 receptor at least 1-fold, 2-fold, 3-fold, 4-fold, 5-fold, 6-fold, 7-fold, 8-fold, 9-fold, 10-fold, 11-fold, 12-fold, 13-fold, 14-fold, or 15-fold stronger than Faricimab inhibits the interaction. In some embodiments, the ANG-2 receptor is a tyrosine kinase with immunoglobulin-like and EGF-like domains 2 receptor (Tie-2 receptor).

In certain embodiments, the antigen binding polypeptides are anti-Human VEGF-C antigen binding polypeptides which bind to human VEGF-C at a K_{D} or EC₅₀ of 100, 75, 60, 50, 40, 30, or 25 picomolar or less. In certain embodiments, the antigen binding polypeptides are anti-Human VEGF-C antigen binding polypeptides which bind to human VEGF at a K_{D} or EC₅₀ of 100, 75, 60, 50, 40, 30, or 25 picomolar or less. In certain embodiments, the antigen binding polypeptides are anti-Human VEGF-C antigen binding polypeptides which bind to human VEGF at a K_{D} or EC₅₀ of 1, 2, 3, 4, 5, 6, 7, 8, 9, or 10 picomolar or more.

In certain embodiments, the antigen binding polypeptides are anti-Human VEGF-A antigen binding polypeptides which inhibits an interaction of VEGF-A or a fragment thereof and a VEGF receptor. In certain embodiments, the antigen binding polypeptides are anti-Human VEGF-A antigen binding polypeptides which inhibits an interaction of VEGF-A or a fragment thereof and a VEGF receptor at an IC₅₀ of 5 nM, 4 nM, 3 nM, 2 nM, 1 nM, 900 pM, 800 pM, 700 pM, 600 pM or less. In certain embodiments, the antigen binding polypeptides are anti-Human VEGF-A antigen binding polypeptides which inhibits an interaction of VEGF-A or a fragment thereof and a VEGF receptor at an IC₅₀ of 1, 2, 3, 4, 5, 6, 7, 8, 9, or 10 picomolar or more. In certain embodiments, the antigen binding polypeptides disclosed herein inhibit the interaction of VEGF_A or a fragment thereof and the VEGF receptor at least 1-fold, 2-fold, 3-fold, 4-fold, 5-fold, 6-fold, 7-fold, 8-fold, 9-fold, or 10-fold stronger than Faricimab inhibits the interaction. In certain embodiments, the VEGF receptor is VEGF receptor 2/kinase insert domain receptor (KDR).

In certain embodiments, the antigen binding polypeptides are anti-Human VEGF-A/ANG-2/VEGF-C trispecific antigen binding polypeptides which bind to human VEGF-A at a K_{D} or EC₅₀ of 100, 75, 60, 50, 40, 30, or 25 picomolar or less. In certain embodiments, the antigen binding polypeptides are anti-Human VEGF/ANG-2 bispecific antigen binding polypeptides which bind to human VEGF-A at a K_{D} or EC₅₀ of 1, 2, 3, 4, 5, 6, 7, 8, 9, or 10 picomolar or more.

In certain embodiments, the antigen binding polypeptides are anti-Human VEGF-A/ANG-2/VEGF-C trispecific antigen binding polypeptides which bind to human ANG-2 at a K_{D} or EC₅₀ of 100, 75, 60, 50, 40, 30, or 25 picomolar or less. In certain embodiments, the antigen binding polypeptides are anti-Human VEGF/ANG-2/VEGF-C trispecific antigen binding polypeptides which bind to human ANG-2 at a K_{D} or EC₅₀ of 1, 2, 3, 4, 5, 6, 7, 8, 9, or 10 picomolar or more.

In certain embodiments, the antigen binding polypeptides are anti-Human VEGF-A/ANG-2/VEGF-C trispecific antigen binding polypeptides which bind to human VEGF-C at a K_{D} or EC₅₀ of 100, 75, 60, 50, 40, 30, or 25 picomolar or less. In certain embodiments, the antigen binding polypeptides are anti-Human VEGF/ANG-2/VEGF-C trispecific antigen binding polypeptides which bind to human VEGF-C at a K_{D} or EC₅₀ of 1, 2, 3, 4, 5, 6, 7, 8, 9, or 10 picomolar or more.

In some embodiments, the antigen binding polypeptides disclosed herein are IgG antigen binding polypeptides. In some embodiments, the antigen binding polypeptides disclosed herein are IgE antigen binding polypeptides. In some embodiments, the antigen binding polypeptides disclosed herein are IgM antigen binding polypeptides. In some embodiments, the antigen binding polypeptides disclosed herein are IgA antigen binding polypeptides. In some embodiments, the antigen binding polypeptides disclosed herein are Fab, F(ab)₂, single-domain antibody, or single chain variable fragment (scFv).

In some embodiments, paired with one or more surrogate light chains, the antigen binding polypeptides disclosed herein are surrobodies. In some embodiments, the antigen binding fragment thereof is a surrobody antigen-binding fragment (Sab) or single chain variable fragment of a surrobody (scSv).

In some embodiments, one or more amino acid modifications may be introduced into the Fc region of an antigen binding polypeptide provided herein, thereby generating an Fc region variant. An Fc region herein is a C-terminal region of an immunoglobulin heavy chain that contains at least a portion of the constant region. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some instances, the Fc region of an immunoglobulin is important for many important antibody functions (e.g. effector functions), such as antigen-dependent cellular cytotoxicity (ADCC), complement dependent cytotoxicity (CDC), and antibody-dependent cell-mediated phagocytosis (ADCP), result in killing of target cells, albeit by different mechanisms. Accordingly, in some embodiments, the antigen binding polypeptides described herein comprise the variable domains of the invention combined with constant domains comprising different Fc regions, selected based on the biological activities of the antibody for the intended use. In certain instances, Human IgGs, for example, can be classified into four subclasses, IgG1, IgG2, IgG3, and IgG4, and each these of these comprises an Fc region having a unique profile for binding to one or more of Fcγ receptors (activating receptors FcγRI (CD64), FcγRIIA, FcγRIIC (CD32); FcγRIIIA and FcγRIIIB (CD16) and inhibiting receptor FcγRIIB), and for the first component of complement (C1q). Human IgG1 and IgG3 bind to all Fcγ receptors; IgG2 binds to FcγRIIA_{H131}, and with lower affinity to FcγRIIA_{R131} FcγRIIIA_{V158}; IgG4 binds to FcγRI, FcγRIIA, FcγRIIB, FcγRIIC, and FcγRIIIA_{V158}; and the inhibitory receptor FcγRIIB has a lower affinity for IgG1, IgG2 and IgG3 than all other Fcγ receptors. Studies have shown that FcγRI does not bind to IgG2, and FcγRIIIB does not bind to IgG2 or IgG4. Id. In general, with regard to ADCC activity, human IgG1≥IgG3>>IgG4≥IgG2.

In some embodiments, the antigen binding polypeptides of this disclosure are variants that possess reduced effector functions, which make it a desirable candidate for applications in which certain effector functions (such as complement fixation and ADCC) are unnecessary or deleterious. Such antibodies can have decreased complement-dependent cytotoxicity (CDC), antibody-dependent cell cytotoxicity (ADCC), or antibody dependent cellular phagocytosis (ADCP). In some embodiments, the antigen binding polypeptides of this disclosure are variants that possess increased effector functions for applications in which increased immunogenicity would be beneficial. Such antigen binding polypeptides can have increased CDC, ADCC, or ADCP, or a combination thereof. Non-limiting examples of in vitro assays to assess ADCC activity of a molecule of interest is described in U.S. Pat. Nos. 5,500,362 and 5,821,337. Alternatively, non-radioactive assays methods may be employed (e.g., ACTI™ and CytoTox 96® non-radioactive cytotoxicity assays). Useful effector cells for such assays include peripheral blood mononuclear cells (PBMC), monocytes, macrophages, and Natural Killer (NK) cells.

Antigen binding polypeptides can have increased half-lives and improved binding to the neonatal Fc receptor (FcRn) (See e.g., US 2005/0014934). Such antigen binding polypeptides can comprise an Fc region with one or more substitutions therein which improve binding of the Fc region to FcRn, and include those with substitutions at one or more of Fc region residues: 238, 256, 265, 272, 286, 303, 305, 307, 311, 312, 317, 340, 356, 360, 362, 376, 378, 380, 382, 413, 424 or 434 according to the EU numbering system (See e.g., U.S. Pat. No. 7,371,826). Other examples of Fc region variants are also contemplated (See e.g., Duncan & Winter, Nature 322:738-40 (1988); U.S. Pat. Nos. 5,648,260 and 5,624,821; and WO94/29351).

In some embodiments, it may be desirable to create cysteine engineered antigen binding polypeptides, e.g., “thioMAbs,” in which one or more residues of an antibody are substituted with cysteine residues. In some embodiments, the substituted residues occur at accessible sites of the antibody. Reactive thiol groups can be positioned at sites for conjugation to other moieties, such as drug moieties or linker drug moieties, to create an immunoconjugate. In some embodiments, any one or more of the following residues may be substituted with cysteine: V205 (Kabat numbering) of the light chain; A118 (EU numbering) of the heavy chain; and 5400 (EU numbering) of the heavy chain Fc region.

In some embodiments, an antigen binding polypeptide provided herein may be further modified to contain additional nonproteinaceous moieties that are known and available. The moieties suitable for derivatization of the antigen binding polypeptide include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone)polyethylene glycol, polypropylene glycol homopolymers, polypropylen oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antigen binding polypeptide may vary, and if two or more polymers are attached, they can be the same or different molecules.

The antigen binding polypeptides described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a genomic region. In certain embodiments, the expression vector is a plasmid. In certain embodiments, the expression vector is a lentivirus, adenovirus, or adeno-associated virus. In certain embodiments, the expression vector is an adenovirus. In certain embodiments, the expression vector is an adeno-associated virus. In certain embodiments, the expression vector is a lentivirus. In certain embodiments, the expression vector described herein is used in a gene therapy. Accordingly, in some embodiments, the expression vector described herein is formulated for intravenous administration. In some embodiments, the expression vector described herein is formulated for intravitreal administration.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

The nucleic acids encoding the antigen binding polypeptides described herein can be used to infect, transfect, transform, or otherwise render a suitable cell transgenic for the nucleic acid, thus enabling the production of antigen binding polypeptides for commercial or therapeutic uses. Standard cell lines and methods for the production of antigen binding polypeptides from a large scale cell culture are known in the art. See e.g., Li et al., “Cell culture processes for monoclonal antibody production.” Mabs. 2010 September-October; 2(5): 466-477. In certain embodiments, the cell is a Eukaryotic cell. In certain embodiments, the Eukaryotic cell is a mammalian cell. In certain embodiments, the mammalian cell is a cell line useful for producing antigen binding polypeptides is a Chines Hamster Ovary cell (CHO) cell, an NS0 murine myeloma cell, or a PER.C6® cell. In certain embodiments, the nucleic acid encoding the antigen binding polypeptide is integrated into a genomic locus of a cell useful for producing antigen binding polypeptides. In certain embodiments, described herein is a method of making an antigen binding polypeptide comprising culturing a cell comprising a nucleic acid encoding an antigen binding polypeptide under conditions in vitro sufficient to allow production and secretion of said antigen binding polypeptide.

In certain embodiments, described herein, is a master cell bank comprising: (a) a mammalian cell line comprising a nucleic acid encoding an antigen binding polypeptide or bispecific antigen binding polypeptide described herein integrated at a genomic location; and (b) a cryoprotectant. In certain embodiments, the cryoprotectant comprises glycerol or DMSO. In certain embodiments, the master cell bank comprises: (a) a CHO cell line comprising a nucleic acid encoding an antigen binding polypeptide or bispecific antigen binding polypeptide described herein integrated at a genomic location; and (b) a cryoprotectant. In certain embodiments, the cryoprotectant comprises glycerol or DMSO. In certain embodiments, the master cell bank is contained in a suitable vial or container able to withstand freezing by liquid nitrogen.

Also described herein are methods of making an antigen binding polypeptide described herein. Such methods comprise incubating a cell or cell-line comprising a nucleic acid encoding the antigen binding polypeptide in a cell culture medium under conditions sufficient to allow for expression and secretion of the antigen binding polypeptide, and further harvesting the antigen binding polypeptide from the cell culture medium. The harvesting can further comprise one or more purification steps to remove live cells, cellular debris, non-antigen binding polypeptide proteins or polypeptides, undesired salts, buffers, and medium components. In certain embodiments, the additional purification step(s) include centrifugation, ultracentrifugation, protein A, protein G, protein A/G, or protein L purification, and/or ion exchange chromatography.

“Treat,” “treatment,” or “treating,” as used herein refers to, e.g., a deliberate intervention to a physiological disease state resulting in the reduction in severity of a disease or condition; the reduction in the duration of a condition course; the amelioration or elimination of one or more symptoms associated with a disease or condition; or the provision of beneficial effects to a subject with a disease or condition. Treatment does not require curing the underlying disease or condition.

A “therapeutically effective amount,” “effective dose,” “effective amount,” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

As used herein, “pharmaceutically acceptable” with reference to a carrier” “excipient” or “diluent” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound, i.e., antibody, can be coated in a material to protect the compound from the action of acids and other natural conditions that can inactivate the compound.

The pharmaceutical compounds described herein can include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

### Angiopoietin-2 (ANG-2) Binding Polypeptide

Described herein is an angiopoietin 2 (ANG-2) antigen binding polypeptide or antigen binding fragment thereof comprising an ANG-2 heavy chain variable region paired to a light chain polypeptide, wherein the ANG-2 heavy chain variable region comprises: a heavy chain complementarity determining region 1 (HCDR1) comprising the amino acid sequence set forth in any one of SEQ ID NOs: 13, 41, 44, and 47; a heavy chain complementarity determining region 2 (HCDR2) comprising the amino acid sequence set forth in any one of SEQ ID NOs: 14, 42, 45, and 48; and a heavy chain complementarity determining region 3 (HCDR3) comprising the amino acid sequence set forth in any one of SEQ ID NOs: 15, 43, 46, and 49.

In some embodiments, the ANG-2 antigen binding polypeptide or antigen binding fragment comprises a heavy chain variable region comprising an amino acid sequence comprising at least 85% identity to the amino acid sequence set forth in SEQ ID NO: 11. In some embodiments, the ANG-2 antigen binding polypeptide or antigen binding fragment comprises a heavy chain variable region comprising an amino acid sequence comprising at least 90% identity to the amino acid sequence set forth in SEQ ID NO: 11. In some embodiments, the ANG-2 antigen binding polypeptide or antigen binding fragment comprises a heavy chain variable region comprising an amino acid sequence comprising at least 95% identity to the amino acid sequence set forth in SEQ ID NO: 11. In some embodiments, the ANG-2 antigen binding polypeptide or antigen binding fragment comprises a heavy chain variable region comprising an amino acid sequence comprising at least 96% identity to the amino acid sequence set forth in SEQ ID NO: 11. In some embodiments, the ANG-2 antigen binding polypeptide or antigen binding fragment comprises a heavy chain variable region comprising an amino acid sequence comprising at least 97% identity to the amino acid sequence set forth in SEQ ID NO: 11. In some embodiments, the ANG-2 antigen binding polypeptide or antigen binding fragment comprises a heavy chain variable region comprising an amino acid sequence comprising at least 98% identity to the amino acid sequence set forth in SEQ ID NO: 11. In some embodiments, the ANG-2 antigen binding polypeptide or antigen binding fragment comprises a heavy chain variable region comprising an amino acid sequence comprising at least 99% identity to the amino acid sequence set forth in SEQ ID NO: 11. In some embodiments, the ANG-2 antigen binding polypeptide or antigen binding fragment comprises a heavy chain variable region comprising an amino acid sequence set forth in SEQ ID NO: 11.

In some embodiments, the ANG-2 antigen binding polypeptide or antigen binding fragment comprises a heavy chain variable region comprising an amino acid sequence comprising at least 85% identity to the amino acid sequence set forth in SEQ ID NO: 12. In some embodiments, the ANG-2 antigen binding polypeptide or antigen binding fragment comprises a heavy chain variable region comprising an amino acid sequence comprising at least 90% identity to the amino acid sequence set forth in SEQ ID NO: 12. In some embodiments, the ANG-2 antigen binding polypeptide or antigen binding fragment comprises a heavy chain variable region comprising an amino acid sequence comprising at least 95% identity to the amino acid sequence set forth in SEQ ID NO: 12. In some embodiments, the ANG-2 antigen binding polypeptide or antigen binding fragment comprises a heavy chain variable region comprising an amino acid sequence comprising at least 96% identity to the amino acid sequence set forth in SEQ ID NO: 12. In some embodiments, the ANG-2 antigen binding polypeptide or antigen binding fragment comprises a heavy chain variable region comprising an amino acid sequence comprising at least 97% identity to the amino acid sequence set forth in SEQ ID NO: 12. In some embodiments, the ANG-2 antigen binding polypeptide or antigen binding fragment comprises a heavy chain variable region comprising an amino acid sequence comprising at least 98% identity to the amino acid sequence set forth in SEQ ID NO: 12. In some embodiments, the ANG-2 antigen binding polypeptide or antigen binding fragment comprises a heavy chain variable region comprising an amino acid sequence comprising at least 99% identity to the amino acid sequence set forth in SEQ ID NO: 12. In some embodiments, the ANG-2 antigen binding polypeptide or antigen binding fragment comprises a heavy chain variable region comprising an amino acid sequence set forth in SEQ ID NO: 12.

In some embodiments, the light chain polypeptide is non-covalently associated with the ANG-2 heavy chain variable region. In some embodiments, the light chain polypeptide is covalently associated with the ANG-2 heavy chain variable region. In some embodiments, the light chain polypeptide is covalently associated with the ANG-2 heavy chain variable region by one or more disulfide bond.

In some embodiments, the light chain polypeptide is a surrogate light chain polypeptide. In some embodiments, the surrogate light chain polypeptide comprises a VpreB sequence conjugated to a λ5 sequence.

In some embodiments, the VpreB sequence comprises an amino acid sequence comprising at least 90% sequence identity to the amino acid sequence set forth in SEQ ID NO: 101. In some embodiments, the VpreB sequence comprises an amino acid sequence comprising at least 95% sequence identity to the amino acid sequence set forth in SEQ ID NO: 101. In some embodiments, the VpreB sequence comprises an amino acid sequence comprising at least 97% sequence identity to the amino acid sequence set forth in SEQ ID NO: 101. In some embodiments, the VpreB sequence comprises an amino acid sequence comprising at least 98% sequence identity to the amino acid sequence set forth in SEQ ID NO: 101. In some embodiments, the VpreB sequence comprises an amino acid sequence comprising at least 99% sequence identity to the amino acid sequence set forth in SEQ ID NO: 101. In some embodiments, the VpreB sequence comprises an amino acid sequence set forth in SEQ ID NO: 101.

In some embodiments, the λ5 sequence comprises an amino acid sequence comprising at least 90% sequence identity to the amino acid sequence set forth in SEQ ID NO: 106. In some embodiments, the λ5 sequence comprises an amino acid sequence comprising at least 95% sequence identity to the amino acid sequence set forth in SEQ ID NO: 106. In some embodiments, the λ5 sequence comprises an amino acid sequence comprising at least 97% sequence identity to the amino acid sequence set forth in SEQ ID NO: 106. In some embodiments, the λ5 sequence comprises an amino acid sequence comprising at least 98% sequence identity to the amino acid sequence set forth in SEQ ID NO: 106. In some embodiments, the λ5 sequence comprises an amino acid sequence comprising at least 99% sequence identity to the amino acid sequence set forth in SEQ ID NO: 106. In some embodiments, the λ5 sequence comprises an amino acid sequence set forth in SEQ ID NO: 106.

In some embodiments, the surrogate light chain polypeptide comprises: a light chain complementarity determining region 1 (LCDR1) comprising the amino acid sequence set forth in SEQ ID NO: 103; a light chain complementarity determining region 2 (LCDR2) comprising the amino acid sequence set forth in SEQ ID NO: 104; and a light chain complementarity determining region 3 (LCDR3) comprising the amino acid sequence set forth in SEQ ID NO: 105.

In some embodiments, the surrogate light chain polypeptide comprises an amino acid sequence comprising at least 85% sequence identity to the amino acid sequence set forth in SEQ ID NO: 102. In some embodiments, the surrogate light chain polypeptide comprises an amino acid sequence comprising at least 90% sequence identity to the amino acid sequence set forth in SEQ ID NO: 102. In some embodiments, the surrogate light chain polypeptide comprises an amino acid sequence comprising at least 95% sequence identity to the amino acid sequence set forth in SEQ ID NO: 102. In some embodiments, the surrogate light chain polypeptide comprises an amino acid sequence comprising at least 96% sequence identity to the amino acid sequence set forth in SEQ ID NO: 102. In some embodiments, the surrogate light chain polypeptide comprises an amino acid sequence comprising at least 97% sequence identity to the amino acid sequence set forth in SEQ ID NO: 102. In some embodiments, the surrogate light chain polypeptide comprises an amino acid sequence comprising at least 98% sequence identity to the amino acid sequence set forth in SEQ ID NO: 102. In some embodiments, the surrogate light chain polypeptide comprises an amino acid sequence comprising at least 99% sequence identity to the amino acid sequence set forth in SEQ ID NO: 102. In some embodiments, the surrogate light chain polypeptide comprises an amino acid sequence set forth in SEQ ID NO: 102.

In some embodiments, the light chain polypeptide is a common light chain polypeptide.

### Multispecific Antigen Binding Polypeptide

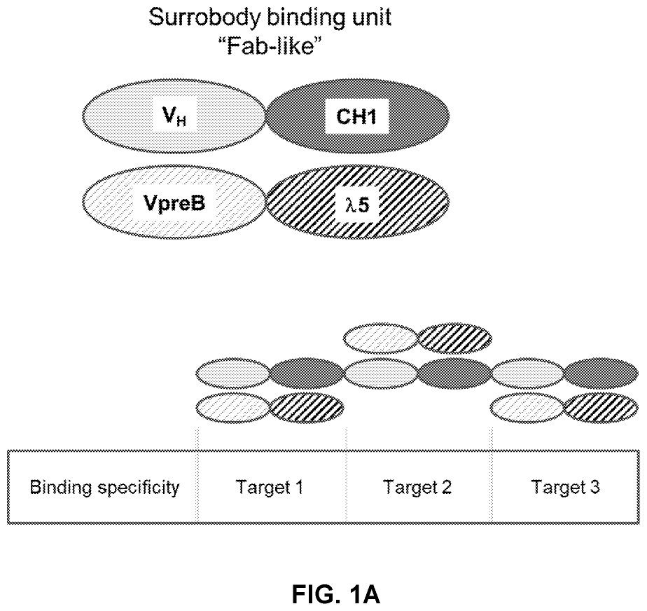

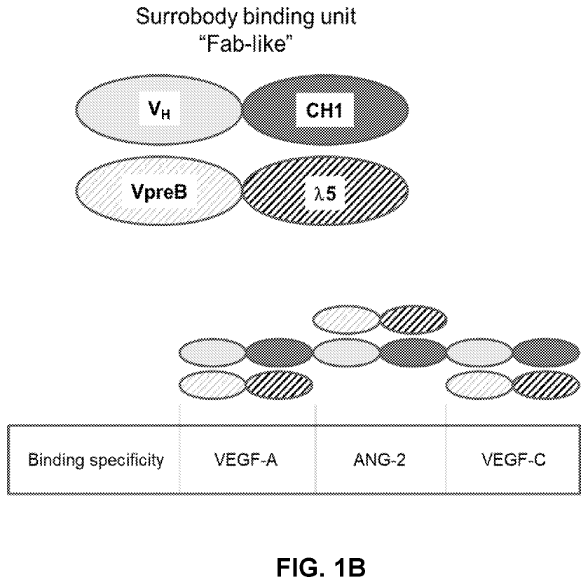

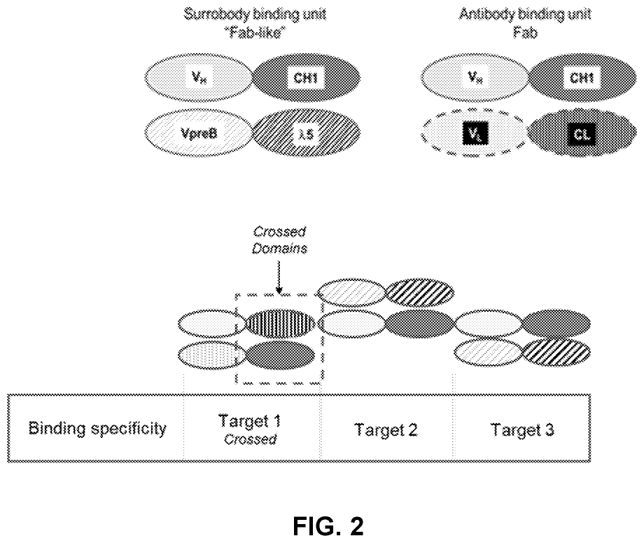

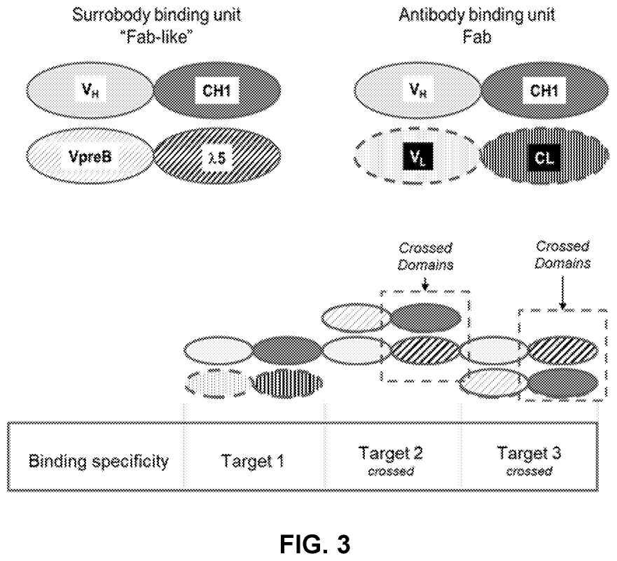

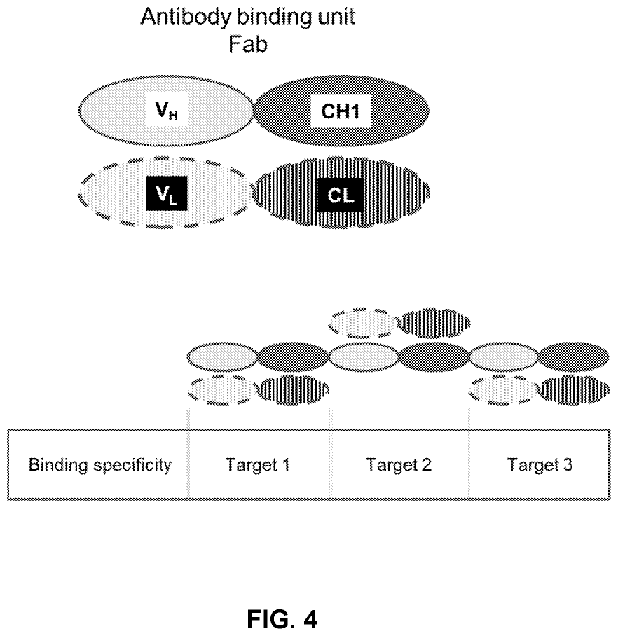

Described herein are multispecific antigen binding polypeptides. Exemplary multispecific antigen binding polypeptides are illustrated in FIGS. 1A, 1B, and 2-4. A trispecific binding polypeptide can comprise a single polypeptide chain comprising three repeats of an antigen binding moiety comprising a heavy chain variable region (VH) and a 1^{st} constant domain of a heavy chain immunoglobulin (CH1). All three VH/CH1s may be the same creating three binding domains for a single antigen, or different creating one binding domain for three different antigens.

In some embodiments, the first antigen binding moiety comprises a crossed domain. In some embodiments, the second antigen binding moiety comprises a crossed domain. In some embodiments, the third antigen binding moiety comprises a crossed domain. In some embodiments, each of the first antigen binding moiety and the second antigen binding moiety comprises a crossed domain. In some embodiments, each of the first antigen binding moiety and the third antigen binding moiety comprises a crossed domain. In some embodiments, each of the second antigen binding moiety and the third antigen binding moiety comprises a crossed domain.

In some embodiments, each of the first antigen binding moiety, the second antigen binding moiety and the third antigen binding moiety is non-covalently paired to the surrogate light chain polypeptide. In some embodiments, each of the first antigen binding moiety, the second antigen binding moiety and the third antigen binding moiety is covalently paired to the surrogate light chain polypeptide. In some embodiments, each of the first antigen binding moiety, the second antigen binding moiety and the third antigen binding moiety is covalently paired to the surrogate light chain polypeptide by one or more disulfide bond.

### VEGF-A/ANG-2/VEGF-C Trispecific Antigen Binding Polypeptide

Described herein in one aspect, is a multispecific antigen binding polypeptide comprising: an ANG-2 heavy chain variable region paired to a light chain polypeptide, wherein the ANG-2 heavy chain variable region comprises: a heavy chain complementarity determining region 1 (HCDR1) comprising the amino acid sequence set forth in any one of SEQ ID NOs: 13, 41, 44, and 47; a heavy chain complementarity determining region 2 (HCDR2) comprising the amino acid sequence set forth in any one of SEQ ID NOs: 14, 42, 45, and 48; and a heavy chain complementarity determining region 3 (HCDR3) comprising the amino acid sequence set forth in any one of SEQ ID NOs: 15, 43, 46, and 49.

In some embodiments, the ANG-2 antigen binding polypeptide or antigen binding fragment comprises a heavy chain variable region comprising an amino acid sequence comprising at least 85% identity to the amino acid sequence set forth in SEQ ID NO: 11. In some embodiments, the ANG-2 antigen binding polypeptide or antigen binding fragment comprises a heavy chain variable region comprising an amino acid sequence comprising at least 90% identity to the amino acid sequence set forth in SEQ ID NO: 11. In some embodiments, the ANG-2 antigen binding polypeptide or antigen binding fragment comprises a heavy chain variable region comprising an amino acid sequence comprising at least 95% identity to the amino acid sequence set forth in SEQ ID NO: 11. In some embodiments, the ANG-2 antigen binding polypeptide or antigen binding fragment comprises a heavy chain variable region comprising an amino acid sequence comprising at least 96% identity to the amino acid sequence set forth in SEQ ID NO: 11. In some embodiments, the ANG-2 antigen binding polypeptide or antigen binding fragment comprises a heavy chain variable region comprising an amino acid sequence comprising at least 97% identity to the amino acid sequence set forth in SEQ ID NO: 11. In some embodiments, the ANG-2 antigen binding polypeptide or antigen binding fragment comprises a heavy chain variable region comprising an amino acid sequence comprising at least 98% identity to the amino acid sequence set forth in SEQ ID NO: 11. In some embodiments, the ANG-2 antigen binding polypeptide or antigen binding fragment comprises a heavy chain variable region comprising an amino acid sequence comprising at least 99% identity to the amino acid sequence set forth in SEQ ID NO: 11. In some embodiments, the ANG-2 antigen binding polypeptide or antigen binding fragment comprises a heavy chain variable region comprising an amino acid sequence set forth in SEQ ID NO: 11.

In some embodiments, the ANG-2 antigen binding polypeptide or antigen binding fragment comprises a heavy chain variable region comprising an amino acid sequence comprising at least 85% identity to the amino acid sequence set forth in SEQ ID NO: 12. In some embodiments, the ANG-2 antigen binding polypeptide or antigen binding fragment comprises a heavy chain variable region comprising an amino acid sequence comprising at least 90% identity to the amino acid sequence set forth in SEQ ID NO: 12. In some embodiments, the ANG-2 antigen binding polypeptide or antigen binding fragment comprises a heavy chain variable region comprising an amino acid sequence comprising at least 95% identity to the amino acid sequence set forth in SEQ ID NO: 12. In some embodiments, the ANG-2 antigen binding polypeptide or antigen binding fragment comprises a heavy chain variable region comprising an amino acid sequence comprising at least 96% identity to the amino acid sequence set forth in SEQ ID NO: 12. In some embodiments, the ANG-2 antigen binding polypeptide or antigen binding fragment comprises a heavy chain variable region comprising an amino acid sequence comprising at least 97% identity to the amino acid sequence set forth in SEQ ID NO: 12. In some embodiments, the ANG-2 antigen binding polypeptide or antigen binding fragment comprises a heavy chain variable region comprising an amino acid sequence comprising at least 98% identity to the amino acid sequence set forth in SEQ ID NO: 12. In some embodiments, the ANG-2 antigen binding polypeptide or antigen binding fragment comprises a heavy chain variable region comprising an amino acid sequence comprising at least 99% identity to the amino acid sequence set forth in SEQ ID NO: 12. In some embodiments, the ANG-2 antigen binding polypeptide or antigen binding fragment comprises a heavy chain variable region comprising an amino acid sequence set forth in SEQ ID NO: 12.

In some embodiments, the light chain polypeptide is non-covalently associated with the ANG-2 heavy chain variable region. In some embodiments, the light chain polypeptide is covalently associated with the ANG-2 heavy chain variable region. In some embodiments, the light chain polypeptide is covalently associated with the ANG-2 heavy chain variable region by one or more disulfide bond.

In some embodiments, the multispecific antigen binding polypeptide comprises a VEGF-A binding moiety. In some embodiments, the VEGF-A binding moiety comprises a VEGF-A heavy chain variable region paired with a light chain polypeptide.

In some embodiments, the VEGF-A heavy chain variable region comprises an immunoglobulin heavy chain variable region comprising: a heavy chain complementarity determining region 1 (HCDR1) comprising the amino acid sequence set forth in SEQ ID NO: 3, 31, 34, and 37; a heavy chain complementarity determining region 2 (HCDR2) comprising the amino acid sequence set forth in SEQ ID NO: 4, 32, 35, and 38; and/or a heavy chain complementarity determining region 3 (HCDR3) comprising the amino acid sequence set forth in SEQ ID NO: 5, 33, 36, and 39.

In some embodiments, the VEGF-A heavy chain variable region comprises an amino acid sequence comprising at least 85% identity to the amino acid sequence set forth in SEQ ID NO: 1. In some embodiments, the VEGF-A heavy chain variable region comprises an amino acid sequence comprising at least 90% identity to the amino acid sequence set forth in SEQ ID NO: 1. In some embodiments, the VEGF-A heavy chain variable region comprises an amino acid sequence comprising at least 95% identity to the amino acid sequence set forth in SEQ ID NO: 1. In some embodiments, the VEGF-A heavy chain variable region comprises an amino acid sequence comprising at least 96% identity to the amino acid sequence set forth in SEQ ID NO: 1. In some embodiments, the VEGF-A heavy chain variable region comprises an amino acid sequence comprising at least 97% identity to the amino acid sequence set forth in SEQ ID NO: 1. In some embodiments, the VEGF-A heavy chain variable region comprises an amino acid sequence comprising at least 98% identity to the amino acid sequence set forth in SEQ ID NO: 1. In some embodiments, the VEGF-A heavy chain variable region comprises an amino acid sequence comprising at least 99% identity to the amino acid sequence set forth in SEQ ID NO: 1. In some embodiments, the VEGF-A heavy chain variable region comprises an amino acid sequence set forth in SEQ ID NO: 1.

In some embodiments, the VEGF-A binding moiety comprises an amino acid sequence comprising at least 85% identity to the amino acid sequence set forth in SEQ ID NO: 2. In some embodiments, the VEGF-A binding moiety comprises an amino acid sequence comprising at least 90% identity to the amino acid sequence set forth in SEQ ID NO: 2. In some embodiments, the VEGF-A binding moiety comprises an amino acid sequence comprising at least 95% identity to the amino acid sequence set forth in SEQ ID NO: 2. In some embodiments, the VEGF-A binding moiety comprises an amino acid sequence comprising at least 96% identity to the amino acid sequence set forth in SEQ ID NO: 2. In some embodiments, the VEGF-A binding moiety comprises an amino acid sequence comprising at least 97% identity to the amino acid sequence set forth in SEQ ID NO: 2. In some embodiments, the VEGF-A binding moiety comprises an amino acid sequence comprising at least 98% identity to the amino acid sequence set forth in SEQ ID NO: 2. In some embodiments, the VEGF-A binding moiety comprises an amino acid sequence comprising at least 99% identity to the amino acid sequence set forth in SEQ ID NO: 2. In some embodiments, the VEGF-A binding moiety comprises an amino acid sequence set forth in SEQ ID NO: 2.

In some embodiments, the light chain polypeptide is non-covalently associated with the VEGF-A heavy chain variable region. In some embodiments, the light chain polypeptide is covalently associated with the VEGF-A heavy chain variable region. In some embodiments, the light chain polypeptide is covalently associated with the VEGF-A heavy chain variable region by one or more disulfide bond.

In some embodiments, the multispecific antigen binding polypeptide comprises a VEGF-C binding moiety. In some embodiments, the VEGF-C binding moiety comprises a VEGF-C heavy chain variable region paired with a light chain polypeptide.

In some embodiments, the VEGF-C heavy chain variable region comprises an immunoglobulin heavy chain variable region comprising: a heavy chain complementarity determining region 1 (HCDR1) comprising the amino acid sequence set forth in SEQ ID NO: 23, 51, 54, and 57; a heavy chain complementarity determining region 2 (HCDR2) comprising the amino acid sequence set forth in SEQ ID NO: 24, 52, 55, and 58; and/or a heavy chain complementarity determining region 3 (HCDR3) comprising the amino acid sequence set forth in SEQ ID NO: 25, 53, 56, and 59.

In some embodiments, the VEGF-C heavy chain variable region comprises an amino acid sequence comprising at least 85% identity to the amino acid sequence set forth in SEQ ID NO: 21. In some embodiments, the VEGF-C heavy chain variable region comprises an amino acid sequence comprising at least 90% identity to the amino acid sequence set forth in SEQ ID NO: 21. In some embodiments, the VEGF-C heavy chain variable region comprises an amino acid sequence comprising at least 95% identity to the amino acid sequence set forth in SEQ ID NO: 21. In some embodiments, the VEGF-C heavy chain variable region comprises an amino acid sequence comprising at least 96% identity to the amino acid sequence set forth in SEQ ID NO: 21. In some embodiments, the VEGF-C heavy chain variable region comprises an amino acid sequence comprising at least 97% identity to the amino acid sequence set forth in SEQ ID NO: 21. In some embodiments, the VEGF-C heavy chain variable region comprises an amino acid sequence comprising at least 98% identity to the amino acid sequence set forth in SEQ ID NO: 21. In some embodiments, the VEGF-C heavy chain variable region comprises an amino acid sequence comprising at least 99% identity to the amino acid sequence set forth in SEQ ID NO: 21. In some embodiments, the VEGF-C heavy chain variable region comprises an amino acid sequence set forth in SEQ ID NO: 21.

In some embodiments, the VEGF-C binding moiety comprises an amino acid sequence comprising at least 85% identity to the amino acid sequence set forth in SEQ ID NO: 22. In some embodiments, the VEGF-C binding moiety comprises an amino acid sequence comprising at least 90% identity to the amino acid sequence set forth in SEQ ID NO: 22. In some embodiments, the VEGF-C binding moiety comprises an amino acid sequence comprising at least 95% identity to the amino acid sequence set forth in SEQ ID NO: 22. In some embodiments, the VEGF-C binding moiety comprises an amino acid sequence comprising at least 96% identity to the amino acid sequence set forth in SEQ ID NO: 22. In some embodiments, the VEGF-C binding moiety comprises an amino acid sequence comprising at least 97% identity to the amino acid sequence set forth in SEQ ID NO: 22. In some embodiments, the VEGF-C binding moiety comprises an amino acid sequence comprising at least 98% identity to the amino acid sequence set forth in SEQ ID NO: 22. In some embodiments, the VEGF-C binding moiety comprises an amino acid sequence comprising at least 99% identity to the amino acid sequence set forth in SEQ ID NO: 22. In some embodiments, the VEGF-C binding moiety comprises an amino acid sequence set forth in SEQ ID NO: 22.

In some embodiments, the light chain polypeptide is non-covalently associated with the VEGF-C heavy chain variable region. In some embodiments, the light chain polypeptide is covalently associated with the VEGF-C heavy chain variable region. In some embodiments, the light chain polypeptide is covalently associated with the VEGF-C heavy chain variable region by one or more disulfide bond.

In some embodiments, the multispecific antigen binding polypeptide comprises an amino acid sequence comprising at least 70% identity to the amino acid sequence set forth in SEQ ID NO: 201. In some embodiments, the multispecific antigen binding polypeptide comprises an amino acid sequence comprising at least 75% identity to the amino acid sequence set forth in SEQ ID NO: 201. In some embodiments, the multispecific antigen binding polypeptide comprises an amino acid sequence comprising at least 80% identity to the amino acid sequence set forth in SEQ ID NO: 201. In some embodiments, the multispecific antigen binding polypeptide comprises an amino acid sequence comprising at least 85% identity to the amino acid sequence set forth in SEQ ID NO: 201. In some embodiments, the multispecific antigen binding polypeptide comprises an amino acid sequence comprising at least 90% identity to the amino acid sequence set forth in SEQ ID NO: 201. In some embodiments, the multispecific antigen binding polypeptide comprises an amino acid sequence comprising at least 95% identity to the amino acid sequence set forth in SEQ ID NO: 201. In some embodiments, the multispecific antigen binding polypeptide comprises an amino acid sequence comprising at least 96% identity to the amino acid sequence set forth in SEQ ID NO: 201. In some embodiments, the multispecific antigen binding polypeptide comprises an amino acid sequence comprising at least 97% identity to the amino acid sequence set forth in SEQ ID NO: 201. In some embodiments, the multispecific antigen binding polypeptide comprises an amino acid sequence comprising at least 98% identity to the amino acid sequence set forth in SEQ ID NO: 201. In some embodiments, the multispecific antigen binding polypeptide comprises an amino acid sequence comprising at least 99% identity to the amino acid sequence set forth in SEQ ID NO: 201. In some embodiments, the multispecific antigen binding polypeptide comprises the amino acid sequence set forth in SEQ ID NO: 201.

In some embodiments, the light chain polypeptide is a common light chain polypeptide. In some embodiments, the light chain polypeptide is a surrogate light chain polypeptide. In some embodiments, the surrogate light chain polypeptide comprises a VpreB sequence conjugated to a λ5 sequence.

In some embodiments, the VpreB sequence comprises an amino acid sequence comprising at least 90% sequence identity to the amino acid sequence set forth in SEQ ID NO: 101. In some embodiments, the VpreB sequence comprises an amino acid sequence comprising at least 95% sequence identity to the amino acid sequence set forth in SEQ ID NO: 101. In some embodiments, the VpreB sequence comprises an amino acid sequence comprising at least 97% sequence identity to the amino acid sequence set forth in SEQ ID NO: 101. In some embodiments, the VpreB sequence comprises an amino acid sequence comprising at least 98% sequence identity to the amino acid sequence set forth in SEQ ID NO: 101. In some embodiments, the VpreB sequence comprises an amino acid sequence comprising at least 99% sequence identity to the amino acid sequence set forth in SEQ ID NO: 101. In some embodiments, the VpreB sequence comprises an amino acid sequence set forth in SEQ ID NO: 101.

In some embodiments, the λ5 sequence comprises an amino acid sequence comprising at least 90% sequence identity to the amino acid sequence set forth in SEQ ID NO: 106. In some embodiments, the λ5 sequence comprises an amino acid sequence comprising at least 95% sequence identity to the amino acid sequence set forth in SEQ ID NO: 106. In some embodiments, the λ5 sequence comprises an amino acid sequence comprising at least 97% sequence identity to the amino acid sequence set forth in SEQ ID NO: 106. In some embodiments, the λ5 sequence comprises an amino acid sequence comprising at least 98% sequence identity to the amino acid sequence set forth in SEQ ID NO: 106. In some embodiments, the λ5 sequence comprises an amino acid sequence comprising at least 99% sequence identity to the amino acid sequence set forth in SEQ ID NO: 106. In some embodiments, the λ5 sequence comprises an amino acid sequence set forth in SEQ ID NO: 106.

In some embodiments, the surrogate light chain polypeptide comprises: a light chain complementarity determining region 1 (LCDR1) comprising the amino acid sequence set forth in SEQ ID NO: 103; a light chain complementarity determining region 2 (LCDR2) comprising the amino acid sequence set forth in SEQ ID NO: 104; and a light chain complementarity determining region 3 (LCDR3) comprising the amino acid sequence set forth in SEQ ID NO: 105.

In some embodiments, the surrogate light chain polypeptide comprises an amino acid sequence comprising at least 85% sequence identity to the amino acid sequence set forth in SEQ ID NO: 102. In some embodiments, the surrogate light chain polypeptide comprises an amino acid sequence comprising at least 90% sequence identity to the amino acid sequence set forth in SEQ ID NO: 102. In some embodiments, the surrogate light chain polypeptide comprises an amino acid sequence comprising at least 95% sequence identity to the amino acid sequence set forth in SEQ ID NO: 102. In some embodiments, the surrogate light chain polypeptide comprises an amino acid sequence comprising at least 96% sequence identity to the amino acid sequence set forth in SEQ ID NO: 102. In some embodiments, the surrogate light chain polypeptide comprises an amino acid sequence comprising at least 97% sequence identity to the amino acid sequence set forth in SEQ ID NO: 102. In some embodiments, the surrogate light chain polypeptide comprises an amino acid sequence comprising at least 98% sequence identity to the amino acid sequence set forth in SEQ ID NO: 102. In some embodiments, the surrogate light chain polypeptide comprises an amino acid sequence comprising at least 99% sequence identity to the amino acid sequence set forth in SEQ ID NO: 102. In some embodiments, the surrogate light chain polypeptide comprises an amino acid sequence set forth in SEQ ID NO: 102.

Described herein, in another aspect, is a multispecific antigen binding polypeptide comprising: (a) a VEGF-C binding moiety, wherein the VEGF-C binding moiety comprises a VEGF-C heavy chain variable region paired to a first surrogate light chain polypeptide; (b) a VEGF-A binding moiety, wherein the VEGF-A binding moiety comprises a VEGF-A heavy chain variable region paired to a second surrogate light chain polypeptide; and (c) an ANG-2 binding moiety, wherein the ANG-2 binding moiety comprises a ANG-2 heavy chain variable region paired to a third surrogate light chain polypeptide; wherein the VEGF-C heavy chain variable region comprises: (a) a heavy chain complementarity determining region 1 (HCDR1) comprising the amino acid sequence set forth in SEQ ID NO: 23, 51, 54, and 57; (b) a heavy chain complementarity determining region 2 (HCDR2) comprising the amino acid sequence set forth in SEQ ID NO: 24, 52, 55, and 58; and/or (c) a heavy chain complementarity determining region 3 (HCDR3) comprising the amino acid sequence set forth in SEQ ID NO: 25, 53, 56, and 59; wherein the VEGF-A heavy chain variable region comprises: a heavy chain complementarity determining region 1 (HCDR1) comprising the amino acid sequence set forth in SEQ ID NO: 3, 31, 34, and 37; a heavy chain complementarity determining region 2 (HCDR2) comprising the amino acid sequence set forth in SEQ ID NO: 4, 32, 35, and 38; and/or a heavy chain complementarity determining region 3 (HCDR3) comprising the amino acid sequence set forth in SEQ ID NO: 5, 33, 36, and 39; wherein the ANG-2 heavy chain variable region comprises: a heavy chain complementarity determining region 1 (HCDR1) comprising the amino acid sequence set forth in any one of SEQ ID NOs: 13, 41, 44, and 47; a heavy chain complementarity determining region 2 (HCDR2) comprising the amino acid sequence set forth in any one of SEQ ID NOs: 14, 42, 45, and 48; and a heavy chain complementarity determining region 3 (HCDR3) comprising the amino acid sequence set forth in any one of SEQ ID NOs: 15, 43, 46, and 49; and wherein the first, second, and third surrogate light chain polypeptide comprises a light chain complementarity determining region 1 (LCDR1) comprising the amino acid sequence set forth in SEQ ID NO: 103; a light chain complementarity determining region 2 (LCDR2) comprising the amino acid sequence set forth in SEQ ID NO: 104; and a light chain complementarity determining region 3 (LCDR3) comprising the amino acid sequence set forth in SEQ ID NO: 105.

In some embodiments, the ANG-2 binding moiety comprises a heavy chain first constant region (CH1). In some embodiments, the VEGF-A binding moiety comprises a heavy chain first constant region (CH1). In some embodiments, the VEGF-C binding moiety comprises a heavy chain first constant region (CH1). In some embodiments, the ANG-2 binding moiety, the VEGF-A binding moiety, and the VEGF-C binding moiety comprises a heavy chain first constant region (CH1).

In some embodiments, the heavy chain first constant region comprises an amino acid sequence with at least 80% identity to the amino acid sequence set forth in SEQ ID NO: 141. In some embodiments, the heavy chain first constant region comprises an amino acid sequence with at least 90% identity to the amino acid sequence set forth in SEQ ID NO: 141. In some embodiments, the heavy chain first constant region comprises an amino acid sequence with at least 95% identity to the amino acid sequence set forth in SEQ ID NO: 141. In some embodiments, the heavy chain first constant region comprises an amino acid sequence with at least 97% identity to the amino acid sequence set forth in SEQ ID NO: 141. In some embodiments, the heavy chain first constant region comprises an amino acid sequence with at least 98% identity to the amino acid sequence set forth in SEQ ID NO: 141. In some embodiments, the heavy chain first constant region comprises an amino acid sequence with at least 99% identity to the amino acid sequence set forth in SEQ ID NO: 141. In some embodiments, the heavy chain first constant region comprises the amino acid sequence set forth in SEQ ID NO: 141.

In some embodiments, the multispecific antigen binding polypeptide comprises an amino acid sequence comprising at least 70% identity to the amino acid sequence set forth in SEQ ID NO: 201. In some embodiments, the multispecific antigen binding polypeptide comprises an amino acid sequence comprising at least 75% identity to the amino acid sequence set forth in SEQ ID NO: 201. In some embodiments, the multispecific antigen binding polypeptide comprises an amino acid sequence comprising at least 80% identity to the amino acid sequence set forth in SEQ ID NO: 201. In some embodiments, the multispecific antigen binding polypeptide comprises an amino acid sequence comprising at least 85% identity to the amino acid sequence set forth in SEQ ID NO: 201. In some embodiments, the multispecific antigen binding polypeptide comprises an amino acid sequence comprising at least 90% identity to the amino acid sequence set forth in SEQ ID NO: 201. In some embodiments, the multispecific antigen binding polypeptide comprises an amino acid sequence comprising at least 95% identity to the amino acid sequence set forth in SEQ ID NO: 201. In some embodiments, the multispecific antigen binding polypeptide comprises an amino acid sequence comprising at least 96% identity to the amino acid sequence set forth in SEQ ID NO: 201. In some embodiments, the multispecific antigen binding polypeptide comprises an amino acid sequence comprising at least 97% identity to the amino acid sequence set forth in SEQ ID NO: 201. In some embodiments, the multispecific antigen binding polypeptide comprises an amino acid sequence comprising at least 98% identity to the amino acid sequence set forth in SEQ ID NO: 201. In some embodiments, the multispecific antigen binding polypeptide comprises an amino acid sequence comprising at least 99% identity to the amino acid sequence set forth in SEQ ID NO: 201. In some embodiments, the multispecific antigen binding polypeptide comprises the amino acid sequence set forth in SEQ ID NO: 201.

In some embodiments, the first, second, and third surrogate light chain polypeptide comprises an amino acid sequence comprising at least 80% sequence identity to the amino acid sequence set forth in SEQ ID NO: 102. In some embodiments, the first, second, and third surrogate light chain polypeptide comprises an amino acid sequence comprising at least 85% sequence identity to the amino acid sequence set forth in SEQ ID NO: 102. In some embodiments, the first, second, and third surrogate light chain polypeptide comprises an amino acid sequence comprising at least 90% sequence identity to the amino acid sequence set forth in SEQ ID NO: 102. In some embodiments, the first, second, and third surrogate light chain polypeptide comprises an amino acid sequence comprising at least 95% sequence identity to the amino acid sequence set forth in SEQ ID NO: 102. In some embodiments, the first, second, and third surrogate light chain polypeptide comprises an amino acid sequence comprising at least 96% sequence identity to the amino acid sequence set forth in SEQ ID NO: 102. In some embodiments, the first, second, and third surrogate light chain polypeptide comprises an amino acid sequence comprising at least 97% sequence identity to the amino acid sequence set forth in SEQ ID NO: 102. In some embodiments, the first, second, and third surrogate light chain polypeptide comprises an amino acid sequence comprising at least 98% sequence identity to the amino acid sequence set forth in SEQ ID NO: 102. In some embodiments, the first, second, and third surrogate light chain polypeptide comprises an amino acid sequence comprising at least 99% sequence identity to the amino acid sequence set forth in SEQ ID NO: 102. In some embodiments, the first, second, and third surrogate light chain polypeptide comprises an amino acid sequence set forth in SEQ ID NO: 102.

In some embodiments, the multispecific antigen binding polypeptide binds to ANG-2. In some embodiments, the first target, the second target and/or the third target is ANG-2 or a fragment thereof. In some embodiments, the multispecific antigen binding polypeptide binds to ANG-2 or a fragment thereof with an EC50 of about 1000, 500, 200, 100, 50, 20, 10, 8, 5, 3, 2, or 1 picomolar or less. In some embodiments, the multispecific antigen binding polypeptide binds to ANG-2 or a fragment thereof with an EC50 of about 99 picomolar or less. In some embodiments, the multispecific antigen binding polypeptide binds to VEGF-A or a fragment thereof at least 1.5-fold, at least 2-fold, at least 5-fold, at least 10-fold, at least 20-fold, at least 50-fold, at least 100-fold, at least 200-fold, or at least 300-fold stronger than faricimab.

In some embodiments, the multispecific antigen binding polypeptide binds to VEGF-A or a fragment thereof. In some embodiments, the multispecific antigen binding polypeptide binds to VEGF-A or a fragment thereof with an EC50 of about 1000, 500, 200, 100, 50, 20, 10, 8, 5, 3, 2, or 1 picomolar or less. In some embodiments, the multispecific antigen binding polypeptide binds to VEGF-A or a fragment thereof with an EC50 of about 19 picomolar or less. In some embodiments, the multispecific antigen binding polypeptide binds to VEGF-A or a fragment thereof at least 2-fold, at least 3-fold, at least 5-fold, at least 8-fold, at least 10-fold, at least 20-fold, or at least 50-fold stronger than faricimab.

In some embodiments, the multispecific antigen binding polypeptide binds to VEGF-C. In some embodiments, the first target, the second target and/or the third target is VEGF-C or a fragment thereof. In some embodiments, the multispecific antigen binding polypeptide binds to VEGF-C or a fragment thereof with an EC50 of about 1000, 500, 200, 100, 50, 20, 10, 8, 5, 3, 2, or 1 picomolar or less. In some embodiments, the multispecific antigen binding polypeptide binds to VEGF-C or a fragment thereof with an EC50 of about 63 picomolar or less.

In some embodiments, the multispecific antigen binding polypeptide inhibits the interaction of ANG-2 and ANG-2 receptor. In some embodiments, the multispecific antigen binding polypeptide inhibits the interaction of ANG-2 and ANG-2 receptor with an IC50 of about 1000, 500, 200, 100, 50, 20, 10, 8, 5, 3, 2, or 1 nanomolar or less. In some embodiments, the multispecific antigen binding polypeptide inhibits the interaction of ANG-2 and ANG-2 receptor with an IC50 of about 13.6 nanomolar or less.

In some embodiments, the multispecific antigen binding polypeptide inhibits the interaction of VEGF-A and VEGF-A receptor. In some embodiments, the multispecific antigen binding polypeptide inhibits the interaction of VEGF-A and VEGF-A receptor with an IC50 of about 1000, 500, 200, 100, 50, 20, 10, 8, 5, 3, 2, or 1 nanomolar or less. In some embodiments, the multispecific antigen binding polypeptide inhibits the interaction of VEGF-A and VEGF-A receptor with an IC50 of about 41 nanomolar or less.

In some embodiments, the multispecific antigen binding polypeptide inhibits the interaction of VEGF-C and VEGF-C receptor. In some embodiments, the multispecific antigen binding polypeptide inhibits the interaction of VEGF-C and VEGF-C receptor with an IC50 of about 1000, 500, 200, 100, 50, 20, 10, 8, 5, 3, 2, or 1 nanomolar or less. In some embodiments, the multispecific antigen binding polypeptide inhibits the interaction of VEGF-C and VEGF-C receptor with an IC50 of about 9.8 nanomolar or less.

For an intravitreal therapeutic, ANG-1 binding is not a favorable attribute. Binding to ANG-1 activates the ANG-1 receptor Tie2, which stabilizes the vasculature and reduces leak in the vascular bed. In addition, Ang-1 increases outflow through the trabecular meshwork and reduces the intraocular pressure in the eye. In non-human primates antibodies that bind Ang-1 cause increase in intraocular pressure through this mechanism. In some embodiments, the VEGF-A/ANG-2/VEGF-C multispecific antigen binding polypeptide does not bind to ANG-1. In some embodiments, the VEGF-A/ANG-2/VEGF-C multispecific antigen binding polypeptide does not inhibit the interaction between ANG-1 and the Tie2 receptor. In some embodiments, the VEGF-A/ANG-2/VEGF-C multispecific antigen binding polypeptide has a much weaker binding affinity to ANG-1 than the VEGF-A/ANG-2/VEGF-C multispecific antigen binding polypeptide RP-019.

Elevated levels of pre-existing anti-drug antibodies (ADAs) in patients may predict adverse outcomes in response to intravitreal injections with anti-vascular endothelial growth factor (anti-VEGF) agents. Higher levels of pre-existing ADAs detected may be correlated with a greater incidence of adverse events such as severe intraocular inflammation (IOI) including occlusive retinal vasculitis upon treatment. In some embodiments, the pre-existing anti-drug antibodies (ADAs) titer in the pooled human serum against the multispecific binding polypeptide is at least 1:100, 1:200, 1:300, 1:400, 1:500, 1:600, 1:800, 1:1000, 1:1500, 1:1800, 1:2000, 1:1500, 1:3000, 1:5000, or 1:10,000. In some embodiments, the pre-existing anti-drug antibodies (ADAs) titer in the pooled human serum against the multispecific binding polypeptide is at least 1:2700.

### Vectors

Some embodiments disclosed herein relate to nucleic acids encoding the ANG-2 binding antibody described herein or the multispecific antigen binding polypeptide as disclosed herein. Some embodiments disclosed herein relate to expression vectors comprising the nucleic acids as disclosed herein. In some instances, the expression vector is an RNA vector. In some instances, the expression vector is plasmid or linearized DNA vector. In some instances, the expression vector is a viral vector. Some embodiments disclosed herein relate to cells comprising the expression vectors as disclosed herein. In some instances, the cell is a eukaryotic cell. In some instances, the cell is a Chinese Hamster Ovary (CHO) cell.

The term “viral vector” is widely used to refer either to a nucleic acid molecule that includes virus-derived nucleic acid elements that typically facilitate transfer of the nucleic acid molecule or integration into the genome of a cell, or to a viral particle that mediates nucleic acid transfer. In some embodiments, the viral vector is an adenovirus. In some embodiments, the viral vector is an adeno-associated virus. In some embodiments, the viral vector is a lentivirus virus.

The nucleic acids described above can be contained within a vector that is capable of directing their expression in, for example, a cell that has been transduced with the vector. Suitable vectors for use in eukaryotic cells are known in the art and are commercially available or readily prepared by a skilled artisan. Additional vectors can also be found, for example, in Ausubel, F. M., et al., Current Protocols in Molecular Biology, (Current Protocol, 1994) and Sambrook et al., “Molecular Cloning: A Laboratory Manual,” 2nd Ed. (1989).

Accordingly, in some instances, the multispecific antigen binding polypeptides of the present disclosure can be expressed from vectors, generally expression vectors. The vectors are useful for autonomous replication in a host cell or may be integrated into the genome of a host cell upon introduction into the host cell, and thereby are replicated along with the host genome (e.g., non-episomal mammalian vectors). Expression vectors are capable of directing the expression of coding sequences to which they are operably linked. In general, expression vectors of utility in recombinant DNA techniques are often in the form of plasmids (vectors). However, other forms of expression vectors, such as viral vectors (e.g., replication defective retroviruses, adenoviruses, and adeno-associated viruses) are also included.

DNA vectors can be introduced into eukaryotic cells via conventional transformation or transfection techniques. Suitable methods for transforming or transfecting host cells can be found in Sambrook et al., (1989) Molecular Cloning: A Laboratory Manual (2nd ed., Cold Spring Harbor Laboratory Press, Plainview, N.Y.) and other standard molecular biology laboratory manuals.

Viral particles typically include viral components, and sometimes also host cell components, in addition to nucleic acid(s). Retroviral vectors used herein contain structural and functional genetic elements, or portions thereof, that are primarily derived from a retrovirus. Retroviral lentivirus vectors contain structural and functional genetic elements, or portions thereof including LTRs, that are primarily derived from a lentivirus (a sub-type of retrovirus).

In some instances, the nucleic acid molecules are delivered by viral or non-viral delivery vehicles known in the art. For example, the nucleic acid molecule can be stably integrated in the host genome, or can be episomally replicating, or present in the recombinant host cell as a mini-circle expression vector for stable or transient expression. Accordingly, in some instances, the nucleic acid molecule is maintained and replicated in the recombinant host cell as an episomal unit. In some instances, the nucleic acid molecule is stably integrated into the genome of the recombinant cell. Stable integration can also be accomplished using classical random genomic recombination techniques or with more precise genome editing techniques such as using guide RNA-directed CRISPR/Cas9, DNA-guided endonuclease genome editing NgAgo (Natronobacterium gregoryi Argonaute), or TALENs genome editing (transcription activator-like effector nucleases). In some instances, the nucleic acid molecule is present in the recombinant host cell as a mini circle expression vector for stable or transient expression.

The nucleic acid molecules can be encapsulated in a viral capsid or a lipid nanoparticle. Alternatively, endonuclease polypeptide(s) can be delivered by viral or non-viral delivery vehicles known in the art, such as electroporation or lipid nanoparticles. For example, introduction of nucleic acids into cells may be achieved using viral transduction methods. In a non-limiting example, adeno-associated virus (AAV) is a non-enveloped virus that can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral systems are also useful for nucleic acid delivery and gene therapy via viral transduction. Lentiviral vectors offer several attractive properties as gene-delivery vehicles, including: (i) sustained gene delivery through stable vector integration into the host cell genome; (ii) the ability to infect both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile (e.g., by targeting a site for integration that has little or no oncogenic potential); and (vii) a relatively easy system for vector manipulation and production.

### Therapeutic Methods

In certain embodiments, the antigen binding polypeptides can be administered to a subject in need thereof by any route suitable for the administration of antigen binding polypeptide-containing pharmaceutical compositions, such as, for example, topically, subcutaneously, intravenously, or intravitreally. In certain embodiments, the antigen binding polypeptide or pharmaceutical composition comprising the antigen binding polypeptide is administered intravenously. In certain embodiments, the antigen binding polypeptide or pharmaceutical composition comprising the antigen binding polypeptide is administered intravitreally. In certain embodiments, the antigen binding polypeptide or pharmaceutical composition comprising the antigen binding polypeptide is administered topically. In certain embodiments, the antigen binding polypeptide or pharmaceutical composition comprising the antigen binding polypeptide is administered subcutaneously.

In certain embodiments, the antigen binding polypeptides are administered on a suitable dosage schedule, for example, weekly, twice weekly, monthly, twice monthly, once every two weeks, once every three weeks, or once a month, once every two months, once every three months, once every four months, once every five months, or once every six months, etc. The antigen binding polypeptides can be administered in any therapeutically effective amount. In certain embodiments, the therapeutically acceptable amount is between about 0.1 mg/kg and about 50 mg/kg. In certain embodiments, the therapeutically acceptable amount is between about 1 mg/kg and about 40 mg/kg. In certain embodiments, the therapeutically acceptable amount is between about 1 mg/kg and about 20 mg/kg. In certain embodiments, the therapeutically acceptable amount is between about 1 mg/kg and about 10 mg/kg. In certain embodiments, the therapeutically acceptable amount is between about 5 mg/kg and about 30 mg/kg. In certain embodiments, the therapeutically acceptable amount is between about 5 mg/kg and about 20 mg/kg. Intravitreal dosage concentrations can range from 50 mg/ml to 150 mg/ml for intravitreal use. Therapeutically effective amounts include amounts sufficient to ameliorate one or more symptoms associated with the disease or affliction to be treated.

In certain embodiments, disclosed herein, are antigen binding polypeptides useful for the treatment of a disease of the eye. In certain embodiments, the disease of the eye is associated with age or another comorbidity such as diabetes mellitus. In certain embodiments, the disease of the eye is macular degeneration. In certain embodiments, the macular degeneration is age related. In certain embodiments, the macular degeneration is diabetes related. In certain embodiments, the macular degeneration is wet macular degeneration. In certain embodiments, the disease of the eye is branch retinal vein occlusion. In certain embodiments, the disease of the eye is central retinal vein occlusion.

In certain embodiments, disclosed herein, are multispecific antigen binding polypeptides useful for the treatment of a cancer.

### Pharmaceutically Acceptable Excipients, Carriers, and Diluents

In certain embodiments, the multispecific antigen binding polypeptides of the current disclosure are included in a pharmaceutical composition comprising one or more pharmaceutically acceptable excipients, carriers, and diluents. Pharmaceutically acceptable excipients, carriers and diluents can be included to increase shelf-life, stability, or the administrability of the antigen binding polypeptide. Such compounds include salts, pH buffers, detergents, anti-coagulants, and preservatives. In certain embodiments, the antigen binding polypeptides of the current disclosure are administered suspended in a sterile solution. In certain embodiments, the solution comprises about 0.9% NaCl. In certain embodiments, the solution comprises about 5.0% dextrose. In certain embodiments, the solution further comprises one or more of buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine, arginine, histidine, or methionine; antioxidants, for example, ascorbic acid, methionine; or chelating agents, for example, EDTA or EGTA.

In certain embodiments, the antigen binding polypeptides of the current disclosure can be shipped/stored lyophilized and reconstituted before administration. In certain embodiments, lyophilized antigen binding polypeptide formulations comprise a bulking agent such as, mannitol, sorbitol, sucrose, trehalose, dextran 40, or combinations thereof. The lyophilized formulation can be contained in a vial comprised of glass or other suitable non-reactive material. The antigen binding polypeptides when formulated, whether reconstituted or not, can be buffered at a certain pH, generally less than 7.0. In certain embodiments, the pH can be between 4.5 and 7.0, 4.5 and 6.5, 4.5 and 6.0, 4.5 and 5.5, 4.5 and 5.0, or 5.0 and 6.0.

Also described herein are kits comprising one or more of the antigen binding polypeptides described herein in a suitable container and one or more additional components selected from: instructions for use; a diluent, an excipient, a carrier, and a device for administration.

In certain embodiments, described herein is a method of preparing a treatment for a disease of the eye comprising admixing one or more pharmaceutically acceptable excipients, carriers, or diluents and an antigen binding polypeptide of the current disclosure. In certain embodiments, described herein is a method of making the ANG-2 binding antibody or the multispecific antigen binding polypeptide described herein comprising a) culturing a cell comprising the nucleic acids described herein under conditions sufficient for expression of the nucleic acids; and b) isolating or purifying the ANG-2 binding antibody or the multispecific antigen binding polypeptide from the cell or a supernatant thereof. In certain embodiments, described herein is a method of preparing a cancer treatment for storage or shipping comprising lyophilizing one or more antigen binding polypeptides of the current disclosure.

### LIST OF THE NUMBERED EMBODIMENTS

Embodiment 1. An Angiopoietin-2 (ANG-2) binding antibody or antigen binding fragment thereof comprising an ANG-2 heavy chain variable region paired to a light chain polypeptide,
wherein the ANG-2 heavy chain variable region comprises:
   a. a heavy chain complementarity determining region 1 (HCDR1) comprising the amino acid sequence set forth in any one of SEQ ID NOs: 13, 41, 44, and 47;
   b. a heavy chain complementarity determining region 2 (HCDR2) comprising the amino acid sequence set forth in any one of SEQ ID NOs: 14, 42, 45, and 48; and
   c. a heavy chain complementarity determining region 3 (HCDR3) comprising the amino acid sequence set forth in any one of SEQ ID NOs: 15, 43, 46, and 49.

Embodiment 2. The ANG-2 binding antibody or antigen binding fragment thereof of embodiment 1, wherein the ANG-2 heavy chain variable region comprises an amino acid sequence comprising at least 85%, 90%, 95%, 97%, 98%, or 99% identity to the amino acid sequence set forth in SEQ ID NO: 11.

Embodiment 3. The ANG-2 binding antibody or antigen binding fragment thereof of embodiment 1 or 2, wherein the ANG-2 heavy chain variable region comprises the amino acid sequence set forth in SEQ ID NO: 11.

Embodiment 4. The ANG-2 binding antibody or antigen binding fragment thereof of any one of embodiments 1-3, wherein the ANG-2 binding moiety comprises an amino acid sequence comprising at least 85%, 90%, 95%, 97%, 98%, or 99% identity to the amino acid sequence set forth in SEQ ID NO: 12.

Embodiment 5. The ANG-2 binding antibody or antigen binding fragment thereof of any one of embodiments 1-3, wherein the ANG-2 binding moiety comprises the amino acid sequence set forth in SEQ ID NO: 12.

Embodiment 6. The ANG-2 binding antibody or antigen binding molecule of any one of embodiments 1-5, wherein the light chain polypeptide is non-covalently associated with the ANG-2 heavy chain variable region.

Embodiment 7. The ANG-2 binding antibody or antigen binding molecule of any one of embodiments 1-5, wherein the light chain polypeptide is covalently associated with the ANG-2 heavy chain variable region.

Embodiment 8. The ANG-2 binding antibody or antigen binding molecule of embodiment 7, wherein the light chain polypeptide is covalently associated with the ANG-2 heavy chain variable region by one or more disulfide bonds.

Embodiment 9. The ANG-2 binding antibody or antigen binding fragment thereof of any one of embodiments 1-8, wherein the light chain polypeptide is a surrogate light chain polypeptide.

Embodiment 10. The ANG-2 binding antibody or antigen binding fragment thereof of embodiment 9, wherein the surrogate light chain polypeptide comprises a VpreB sequence fused to a λ5 sequence.

Embodiment 11. The ANG-2 binding antibody or antigen binding fragment thereof of embodiment 10, wherein the VpreB sequence comprises an amino acid sequence comprising at least 80%, at least 85%, at least 90%, at least 95%, at least 97%, at least 98%, at least 99%, or at least 100% sequence identity to the amino acid sequence set forth in SEQ ID NO: 101.

Embodiment 12. The ANG-2 binding antibody or antigen binding fragment thereof of any one of embodiments 10-11, wherein the λ5 sequence comprises an amino acid sequence comprising at least 80%, at least 85%, at least 90%, at least 95%, at least 97%, at least 98%, at least 99%, or at least 100% sequence identity to the amino acid sequence set forth in SEQ ID NO: 106.

Embodiment 13. The ANG-2 binding antibody or antigen binding fragment thereof of any one of embodiments 9-12, wherein the surrogate light chain polypeptide comprises:
a. a light chain complementarity determining region 1 (LCDR1) comprising the amino acid sequence set forth in SEQ ID NO: 103;
b. a light chain complementarity determining region 2 (LCDR2) comprising the amino acid sequence set forth in SEQ ID NO: 104; and
c. a light chain complementarity determining region 3 (LCDR3) comprising the amino acid sequence set forth in SEQ ID NO: 105.

Embodiment 14. The ANG-2 binding antibody or antigen binding fragment thereof of any one of embodiments 9-13, wherein the surrogate light chain polypeptide comprises an amino acid sequence comprising at least 80%, at least 85%, at least 90%, at least 95%, at least 97%, at least 98%, or at least 99% identity to the amino acid sequence set forth in SEQ ID NO: 102.

Embodiment 15. The ANG-2 binding antibody or antigen binding fragment thereof of any one of embodiments 9-13, wherein the surrogate light chain polypeptide comprises the amino acid sequence set forth in SEQ ID NO: 102.

Embodiment 16. The ANG-2 binding antibody or antigen binding fragment thereof of any one of embodiments 1-5, wherein the light chain polypeptide is a common light chain polypeptide.

Embodiment 17. A multispecific antigen binding molecule comprising an ANG-2 binding moiety, wherein the ANG-2 binding moiety comprises an ANG-2 heavy chain variable region paired to a light chain polypeptide;
wherein the ANG-2 heavy chain variable region comprises:
a. a heavy chain complementarity determining region 1 (HCDR1) comprising the amino acid sequence set forth in any one of SEQ ID NOs: 13, 41, 44, and 47;
b. a heavy chain complementarity determining region 2 (HCDR2) comprising the amino acid sequence set forth in any one of SEQ ID NOs: 14, 42, 45, and 48; and
c. a heavy chain complementarity determining region 3 (HCDR3) comprising the amino acid sequence set forth in any one of SEQ ID NOs: 15, 43, 46, and 49.

Embodiment 18. The multispecific antigen binding molecule of embodiment 17, wherein the ANG-2 heavy chain variable region comprises an amino acid sequence comprising at least 85%, 90%, 95%, 97%, 98%, or 99% identity to the amino acid sequence set forth in SEQ ID NO: 11.

Embodiment 19. The multispecific antigen binding molecule of embodiment 17, wherein the ANG-2 heavy chain variable region comprises the amino acid sequence set forth in SEQ ID NO: 11.

Embodiment 20. The multispecific antigen binding molecule of any one of embodiments 17-19, wherein the ANG-2 binding moiety comprises an amino acid sequence comprising at least 85%, 90%, 95%, 97%, 98%, or 99% identity to the amino acid sequence set forth in SEQ ID NO: 12.

Embodiment 21. The multispecific antigen binding molecule of any one of embodiments 17-19, wherein the ANG-2 binding moiety comprises the amino acid sequence set forth in SEQ ID NO: 12.

Embodiment 22. The multispecific antigen binding molecule of any one of embodiments 17-21, wherein the light chain polypeptide is non-covalently associated with the ANG-2 heavy chain variable region.

Embodiment 23. The multispecific antigen binding molecule of any one of embodiments 17-21, wherein the light chain polypeptide is covalently associated with the ANG-2 heavy chain variable region.

Embodiment 24. The multispecific antigen binding molecule of embodiment 23, wherein the light chain polypeptide is covalently associated with the ANG-2 heavy chain variable region by one or more disulfide bonds.

Embodiment 25. The multispecific antigen binding molecule of any one of embodiments 17-24, comprising a VEGF-A binding moiety, wherein the VEGF-A binding moiety comprises a VEGF-A heavy chain variable region paired with a light chain polypeptide.

Embodiment 26. The multispecific antigen binding molecule of embodiment 25, wherein the VEGF-A heavy chain variable region comprises:
a. a heavy chain complementarity determining region 1 (HCDR1) comprising the amino acid sequence set forth in any one of SEQ ID NOs: 3, 31, 34, and 37;
b. a heavy chain complementarity determining region 2 (HCDR2) comprising the amino acid sequence set forth in any one of SEQ ID NOs: 4, 32, 35, and 38; and
c. a heavy chain complementarity determining region 3 (HCDR3) comprising the amino acid sequence set forth in any one of SEQ ID NOs: 5, 33, 36, and 39.

Embodiment 27. The multispecific antigen binding molecule of any one of embodiments 25-26, wherein the VEGF-A heavy chain variable region comprises an amino acid sequence comprising at least 85%, 90%, 95%, 97%, 98%, or 99% identity to the amino acid sequence set forth in SEQ ID NO: 1.

Embodiment 28. The multispecific antigen binding molecule of any one of embodiments 25-26, wherein the VEGF-A heavy chain variable region comprises the amino acid sequence set forth in SEQ ID NO: 1.

Embodiment 29. The multispecific antigen binding molecule of any one of embodiments 25-28, wherein the VEGF-A binding moiety comprises an amino acid sequence comprising at least 85%, 90%, 95%, 97%, 98%, or 99% identity to the amino acid sequence set forth in SEQ ID NO: 2.

Embodiment 30. The multispecific antigen binding molecule of any one of embodiments 25-28, wherein the VEGF-A binding moiety comprises the amino acid sequence set forth in SEQ ID NO: 2.

Embodiment 31. The multispecific antigen binding molecule of any one of embodiments 25-30, wherein the light chain polypeptide is non-covalently associated with the VEGF-A heavy chain variable region.

Embodiment 32. The multispecific antigen binding molecule of any one of embodiments 25-30, wherein the light chain polypeptide is covalently associated with the VEGF-A heavy chain variable region.

Embodiment 33. The multispecific antigen binding molecule of embodiment 32, wherein the light chain polypeptide is covalently associated with the VEGF-A heavy chain variable region by one or more disulfide bonds.

Embodiment 34. The multispecific antigen binding molecule of any one of embodiments 17-33, comprising a VEGF-C binding moiety, wherein the VEGF-C binding moiety comprises a VEGF-C heavy chain variable region paired with a light chain polypeptide.

Embodiment 35. The multispecific antigen binding molecule of embodiment 34, wherein the VEGF-C heavy chain variable region comprises:
a. a heavy chain complementarity determining region 1 (HCDR1) comprising the amino acid sequence set forth in any one of SEQ ID NOs: 23, 51, 54, and 57;
b. a heavy chain complementarity determining region 2 (HCDR2) comprising the amino acid sequence set forth in any one of SEQ ID NOs: 24, 52, 55, and 58; and
c. a heavy chain complementarity determining region 3 (HCDR3) comprising the amino acid sequence set forth in any one of SEQ ID NOs: 25, 53, 56, and 59.

Embodiment 36. The multispecific antigen binding molecule of any one of embodiments 34-35, wherein the VEGF-C heavy chain variable region comprises an amino acid sequence comprising at least 85%, 90%, 95%, 97%, 98%, or 99% identity to the amino acid sequence set forth in SEQ ID NO: 21.

Embodiment 37. The multispecific antigen binding molecule of any one of embodiments 34-35, wherein the VEGF-C heavy chain variable region comprises the amino acid sequence set forth in SEQ ID NO: 21.

Embodiment 38. The multispecific antigen binding molecule of any one of embodiments 34-37, wherein the VEGF-C binding moiety comprises an amino acid sequence comprising at least 85%, 90%, 95%, 97%, 98%, or 99% identity to the amino acid sequence set forth in SEQ ID NO: 22.

Embodiment 39. The multispecific antigen binding molecule of any one of embodiments 34-37, wherein the VEGF-C binding moiety comprises the amino acid sequence set forth in SEQ ID NO: 22.

Embodiment 40. The multispecific antigen binding molecule of any one of embodiments 34-39, wherein the light chain polypeptide is non-covalently associated with the VEGF-C heavy chain variable region.

Embodiment 41. The multispecific antigen binding molecule of any one of embodiments 34-39, wherein the light chain polypeptide is covalently associated with the VEGF-C heavy chain variable region.

Embodiment 42. The multispecific antigen binding molecule of embodiment 41, wherein the light chain polypeptide is covalently associated with the VEGF-C heavy chain variable region by one or more disulfide bonds.

Embodiment 43. The multispecific antigen binding molecule of any one of embodiments 17-42, wherein the multispecific antigen binding molecule comprises an amino acid sequence comprising at least 85%, 90%, 95%, 97%, 98%, or 99% identity to the amino acid sequence set forth in SEQ ID NO: 201.

Embodiment 44. The multispecific antigen binding molecule of any one of embodiments 17-42, wherein the multispecific antigen binding molecule comprises the amino acid sequence set forth in SEQ ID NO: 201.

Embodiment 45. The multispecific antigen binding molecule of any one of embodiments 17-44, wherein the light chain polypeptide is a common light chain polypeptide.

Embodiment 46. The multispecific antigen binding molecule of any one of embodiments 17-44, wherein the light chain polypeptide is a surrogate light chain polypeptide.

Embodiment 47. The multispecific antigen binding molecule of embodiment 46, wherein the surrogate light chain polypeptide comprises a VpreB sequence fused to a λ5 sequence.

Embodiment 48. The multispecific antigen binding molecule of embodiment 47, wherein the VpreB sequence comprises an amino acid sequence comprising at least 80%, at least 85%, at least 90%, at least 95%, at least 97%, at least 98%, at least 99%, or at least 100% sequence identity to the amino acid sequence set forth in SEQ ID NO: 101.

Embodiment 49. The multispecific antigen binding molecule of any one of embodiments 47-48, wherein the λ5 sequence comprises an amino acid sequence comprising at least 80%, at least 85%, at least 90%, at least 95%, at least 97%, at least 98%, at least 99%, or at least 100% sequence identity to the amino acid sequence set forth in SEQ ID NO: 106.

Embodiment 50. The multispecific antigen binding molecule of any one of embodiments 46-49, wherein the surrogate light chain polypeptide comprises:
a. a light chain complementarity determining region 1 (LCDR1) comprising the amino acid sequence set forth in SEQ ID NO: 103;
b. a light chain complementarity determining region 2 (LCDR2) comprising the amino acid sequence set forth in SEQ ID NO: 104; and
c. a light chain complementarity determining region 3 (LCDR3) comprising the amino acid sequence set forth in SEQ ID NO: 105.

Embodiment 51. The multispecific antigen binding molecule of any one of embodiments 17-50, wherein the surrogate light chain polypeptide comprises an amino acid sequence comprising at least 80%, at least 85%, at least 90%, at least 95%, at least 97%, at least 98%, or at least 99% identity to the amino acid sequence set forth in SEQ ID NO: 102.

Embodiment 52. The multispecific antigen binding molecule of any one of embodiments 17-50, wherein the surrogate light chain polypeptide comprises the amino acid sequence set forth in SEQ ID NO: 102.

Embodiment 53. A multispecific antigen binding molecule comprising: (a) a VEGF-C binding moiety, wherein the VEGF-C binding moiety comprises a VEGF-C heavy chain variable region paired to a first surrogate light chain polypeptide; (b) a VEGF-A binding moiety, wherein the VEGF-A binding moiety comprises a VEGF-A heavy chain variable region paired to a second surrogate light chain polypeptide; and (c) an ANG-2 binding moiety, wherein the ANG-2 binding moiety comprises a ANG-2 heavy chain variable region paired to a third surrogate light chain polypeptide;

wherein the VEGF-C heavy chain variable region comprises:
a. a heavy chain complementarity determining region 1 (HCDR1) comprising the amino acid sequence set forth in any one of SEQ ID NOs: 23, 51, 54, and 57;
b. a heavy chain complementarity determining region 2 (HCDR2) comprising the amino acid sequence set forth in any one of SEQ ID NOs: 24, 52, 55, and 58; and
c. a heavy chain complementarity determining region 3 (HCDR3) comprising the amino acid sequence set forth in any one of SEQ ID NOs: 25, 53, 56, and 59;
   wherein the VEGF-A heavy chain variable region comprises:
a. a heavy chain complementarity determining region 1 (HCDR1) comprising the amino acid sequence set forth in any one of SEQ ID NOs: 3, 31, 34, and 37;
b. a heavy chain complementarity determining region 2 (HCDR2) comprising the amino acid sequence set forth in any one of SEQ ID NOs: 4, 32, 35, and 38; and
c. a heavy chain complementarity determining region 3 (HCDR3) comprising the amino acid sequence set forth in any one of SEQ ID NOs: 5, 33, 36, and 39;
   wherein the ANG-2 heavy chain variable region comprises:
a. a heavy chain complementarity determining region 1 (HCDR1) comprising the amino acid sequence set forth in any one of SEQ ID NOs: 13, 41, 44, and 47;
b. a heavy chain complementarity determining region 2 (HCDR2) comprising the amino acid sequence set forth in any one of SEQ ID NOs: 14, 42, 45, and 48; and
c. a heavy chain complementarity determining region 3 (HCDR3) comprising the amino acid sequence set forth in any one of SEQ ID NOs: 15, 43, 46, and 49;
   and wherein the first, second, and third surrogate light chain polypeptide comprises:
a. a light chain complementarity determining region 1 (LCDR1) comprising the amino acid sequence set forth in SEQ ID NO: 103;
b. a light chain complementarity determining region 2 (LCDR2) comprising the amino acid sequence set forth in SEQ ID NO: 104; and
c. a light chain complementarity determining region 3 (LCDR3) comprising the amino acid sequence set forth in SEQ ID NO: 105.

Embodiment 54. The multispecific antigen binding molecule of any one of embodiments 34-53, wherein the ANG-2 binding moiety, the VEGF-A binding moiety, and/or the VEGF-C binding moiety comprises a heavy chain first constant region (CH1).

Embodiment 55. The multispecific antigen binding molecule of embodiment 54, wherein the heavy chain first constant region comprises an amino acid sequence with at least 80%, at least 90%, at least 95%, at least 97%, at least 98%, at least 99%, or at least 100% identity to the amino acid sequence set forth in SEQ ID NO: 141.

Embodiment 56. The multispecific antigen binding molecule of any one of embodiments 53-55, wherein the multispecific antigen binding molecule comprises an amino acid sequence comprising at least 85%, at least 90%, at least 95%, at least 97%, at least 98%, or at least 99% identity to the amino acid sequence set forth in SEQ ID NO: 201.

Embodiment 57. The multispecific antigen binding molecule of any one of embodiments 53-55, wherein the multispecific antigen binding molecule comprises the amino acid sequence set forth in SEQ ID NO: 201.

Embodiment 58. The multispecific antigen binding molecule of any one of embodiments 53-57, wherein the first, second, and third surrogate light chain polypeptide comprises an amino acid sequence comprising at least 85%, at least 90%, at least 95%, at least 97%, at least 98%, or at least 99% identity to the amino acid sequence set forth in SEQ ID NO: 102.

Embodiment 59. The multispecific antigen binding molecule of any one of embodiments 53-57, wherein the first, second, and third surrogate light chain polypeptide comprises the amino acid sequence set forth in SEQ ID NO: 102.

Embodiment 60. The multispecific antigen binding molecule of any one of embodiments 17-59, wherein the multispecific antigen binding molecule binds to ANG-2 or a fragment thereof with an EC50 of about 99 picomolar or less.

Embodiment 61. The multispecific antigen binding molecule of any one of embodiments 17-60, wherein the multispecific antigen binding molecule binds to VEGF-A or a fragment thereof with an EC50 of about 19 picomolar or less.

Embodiment 62. The multispecific antigen binding molecule of any one of embodiments 17-61, wherein the multispecific antigen binding molecule binds to VEGF-C or a fragment thereof with an EC50 of about 63 picomolar or less.

Embodiment 63. The multispecific antigen binding molecule of any one of embodiments 17-62, wherein the multispecific antigen binding molecule inhibits the interaction of ANG-2 and the ANG-2 receptor with an IC50 of about 13.6 nanomolar or less.

Embodiment 64. The multispecific antigen binding molecule of any one of embodiments 17-63, wherein the multispecific antigen binding molecule inhibits the interaction of VEGF-A and the VEGF-A receptor 2 with an IC50 of about 41 nanomolar or less.

Embodiment 65. The multispecific antigen binding molecule of any one of embodiments 17-64, wherein the multispecific antigen binding molecule inhibits the interaction of VEGF-C and the VEGF-C receptor with an IC50 of about 9.8 nanomolar or less.

Embodiment 66. The multispecific antigen binding molecule of any one of embodiments 17-65, wherein the pre-existing anti-drug antibodies (ADAs) titer in the pooled human serum against the multispecific binding polypeptide is less than 1:2700.

Embodiment 67. A nucleic acid or plurality of nucleic acids encoding the ANG-2 binding antibody or antigen binding fragment thereof of any one of embodiments 1-16 or the multispecific antigen binding molecule of any one of embodiments 17-66.

Embodiment 68. An expression vector comprising the nucleic acid of embodiment 67.

Embodiment 69. The expression vector of embodiment 68, wherein the expression vector is RNA.

Embodiment 70. The expression vector of embodiment 68, wherein the expression vector is plasmid or linearized DNA.

Embodiment 71. The expression vector of embodiment 68, wherein the expression vector is a viral vector.

Embodiment 72. The expression vector of embodiment 71, wherein the viral vector is an adenovirus.

Embodiment 73. The expression vector of embodiment 71, wherein the viral vector is an adeno-associated virus.

Embodiment 74. The expression vector of embodiment 71, wherein the viral vector is a lentivirus virus.

Embodiment 75. A cell comprising the expression vector of any one of embodiments 68-74.

Embodiment 76. The cell of embodiment 75, wherein the cell is eukaryotic.

Embodiment 77. The cell of embodiment 75, wherein the cell is Chinese Hamster Ovary (CHO) cell.

Embodiment 78. A pharmaceutical composition comprising the ANG-2 binding antibody or antigen binding fragment thereof of any one of embodiments 1-16, the multispecific antigen binding molecule of any one of embodiments 17-66 or the nucleic acid or plurality of nucleic acids encoding the multispecific antigen binding molecule of any one of embodiments 67 to 74 and a pharmaceutically acceptable carrier, excipient, or diluent.

Embodiment 79. The pharmaceutical composition of embodiment 78 formulated for intravenous administration.

Embodiment 80. The pharmaceutical composition of embodiment 78 formulated for intravitreal administration.

Embodiment 81. A method of treating an ocular disease in an individual comprising administering to the individual the ANG-2 binding antibody or antigen binding fragment thereof of any one of embodiments 1-16 or the multispecific antigen binding molecule of any one of embodiments 17-66, thereby treating the ocular disease.

Embodiment 82. The method of embodiment 81, wherein the ocular disease comprises exudative macular degeneration, diabetic macular edema, or retinal vein occlusion.

Embodiment 83. A method of making the ANG-2 binding antibody or antigen binding fragment thereof of any one of embodiments 1-16 or the multispecific antigen binding molecule or the antigen binding molecule of any one of embodiments 17-66, comprising culturing a cell comprising the nucleic acid of embodiment 67 under conditions sufficient for expression of the nucleic acid and isolating or purifying the ANG-2 binding antibody or antigen binding fragment thereof or the multispecific antigen binding molecule or the antigen binding molecule from the cell or a supernatant there of.

Embodiment 84. The multispecific antigen binding molecule of embodiment 53, wherein the multispecific antigen binding molecule comprises from N-terminus to C-terminus the VEGF-A binding moiety, the ANG-2 binding moiety, and the VEGF-C binding moiety.

Embodiment 85. The method of embodiment 82, wherein the macular degeneration is wet neovascular macular degeneration.

### EXAMPLES

The following illustrative examples are representative of embodiments of compositions and methods described herein and are not meant to be limiting in any way.

### Example 1—Production of VEGF-A/ANG-2/VEGF-C Trispecific Antigen Binding Polypeptides

The nucleic acid sequence encoding polypeptide corresponding to SEQ ID NO: 201 and SEQ ID NO: 102 were cloned into plasmid vectors to direct eukaryotic cell expression. Protein expression vectors were co-transfected into HEK293 cells.

Transfectants were isolated from supernatants by Protein A chromatography or CaptureSelect IgG-CH1 resin. Purified proteins were respectively analyzed for uniformity, purity, and quantity by SEC-HPLC, SDS-PAGE and UV spectrophotometric analysis.

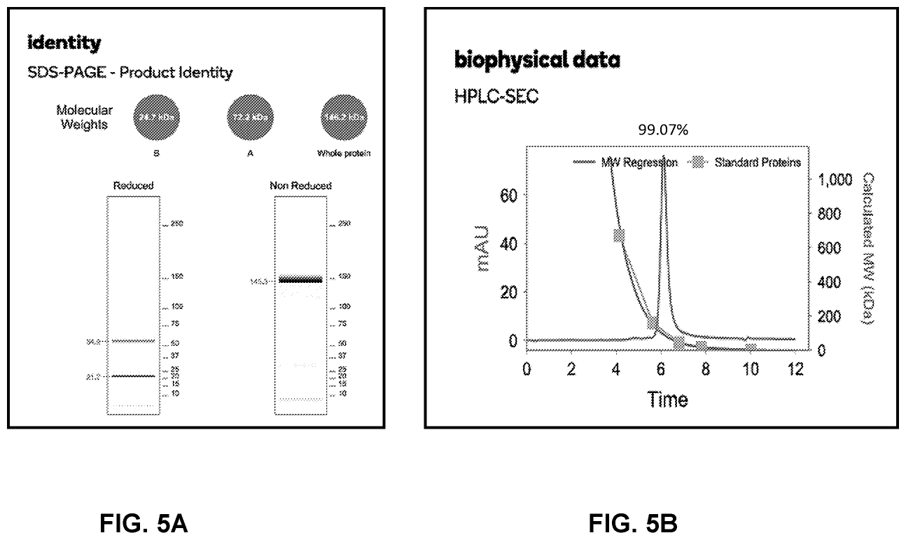

For SDS-PAGE analysis, reduced and non-reduced samples of the RP-043 (SEQ ID NO: 201) were electrophoresed. FIG. 5A confirms the presence of products and good levels of purity were observed. Under non-reducing conditions, protein bands around 145.3 kDa were seen. Under reducing conditions, bands corresponding to the molecular weight of light chain (~21.2 kDa) and heavy chains (~54.9 kDa) were observed. The purified RP-043 proteins were also analyzed in SE-HPLC. The sample proteins showed 99.07% single peak, indicating 99.07% purity (see FIG. 5B).

### Example 2—Binding of RP-043 VEGF-A/ANG-2/VEGF-C Trispecific Antigen Polypeptide

The nucleic acid sequences encoding polypeptides corresponding to SEQ ID NO: 201 and SEQ ID NO: 202 were cloned into plasmid vectors to direct eukaryotic cell expression. Trispecific antigen polypeptides RP-043 (SEQ ID NO: 201) and RP-019 (SEQ ID NO: 202) prepared according to example 1 were tested for their binding affinity to target antigen.

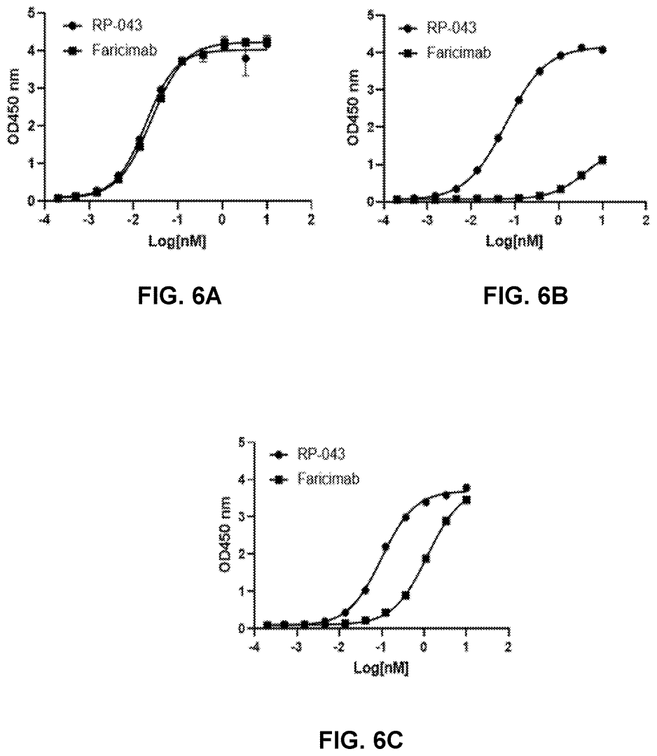

VEGF-A ELISA—Recombinant human VEGF-A (Peprotech, Cat #100-20) was used to coat plates at 1 μg/mL. The products were diluted in blocking buffer at a starting concentration of 10 nM and then 1:3 serial dilutions were tested. The binding was detected by biotinylated anti-IgG-CH1 antibody (ThermoFisher, Cat #7103202500) and HRP-conjugated streptavidin (Jackson ImmunoReserach Lab., Cat #016-030-084). Faricimab were run alongside RP-043 as reference material. OD450 nm readings were measured by Victor 3 (PerkinElmer 1420 Multilabel Counter). As illustrated in FIG. 6A and Table 1, the results showed that RP-043 has a similar binding affinity with EC₅₀ value at 18.9 pM to faricimab with EC₅₀ value at 25 pM.

VEGF-C ELISA Recombinant human VEGF-C (Peprotech, Cat #100-20CD) was used to coat plates at 1 μg/mL. The products were diluted in blocking buffer at a starting concentration of 10 nM and then 1:3 serial dilutions were tested. The binding was detected by biotinylated anti-IgG-CH1 antibody (ThermoFisher, Cat #7103202500) and HRP-conjugated streptavidin (Jackson ImmunoReserach Lab., Cat #016-030-084). Faricimab were run alongside RP-043 as reference material OD450 nm readings were measured by Victor 3 (PerkinElmer 1420 Multilabel Counter). As illustrated in FIG. 6B and Table 1, the results showed that RP-043 has a high binding affinity with EC₅₀ value at 62.8 pM, while faricimab has a much lower binding affinity (EC50 >10 nM). When using medium binding plate (Corning Cat #9017), faricimab did not show binding to rhVEGF-C with either HRP-donkey anti-human IgG-Fcγ (Jackson ImmunoResearch. Labs, Cat #709-035-098) or biotinylated anti-IgG-CH1/HRP-streptavidin (Jackson ImmunoResearch Lab, Cat #016-030-084).

ANG-2 ELISA Recombinant human_ANG-2 (Acro Biosystems, Cat #AN2-H52H4) was used to coat plates at 1 μg/mL. The products were diluted in blocking buffer at a starting concentration of 10 nM and then 1:3 serial dilutions were tested. The binding was detected by biotinylated anti-IgG-CH1 antibody (ThermoFisher, Cat #7103202500) and HRP-conjugated streptavidin (Jackson ImmunoReserach Lab., Cat #016-030-084). Faricimab were run alongside RP-043 as reference material. OD450 nm readings were measured by Victor 3 (PerkinElmer 1420 Multilabel Counter). As illustrated in FIG. 6C and Table 1, the results showed that RP-043 has a 12-fold stronger binding affinity with EC₅₀ value at 99 pM than faricimab with EC₅₀ value at 1201 pM.

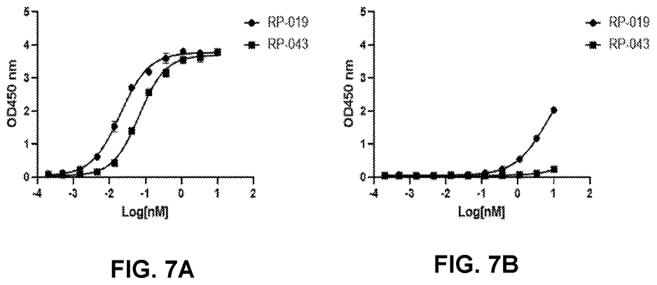

Recombinant human_ANG-2 (Acro Biosystems, Cat #AN2-H52H4) was used to coat plates at 1 μg/mL. The products were diluted in blocking buffer at a starting concentration of 10 nM and then 1:3 serial dilutions were tested. The binding was detected by biotinylated ant-VpreB antibody and HRP-conjugated streptavidin (Jackson ImmunoReserach Lab., Cat #016-030-084). OD450 nm readings were measured by Victor 3 (PerkinElmer 1420 Multilabel Counter). As illustrated in FIG. 7A and Table 1, RP-043 has a strong binding affinity with EC₅₀ value at 66.7 pM, comparable to RP-019.

ANG-1 ELISA Recombinant human_ANG-1 (R&D Systems Cat #923-AN-025/CF was used to coat plates at 1 μg/mL. The products were diluted in blocking buffer at a starting concentration of 10 nM and then 1:3 serial dilutions were tested. The binding was detected by biotinylated anti-VpreB antibody and HRP-conjugated streptavidin (Jackson ImmunoReserach Lab., Cat #016-030-084). OD450 nm readings were measured by Victor 3 (PerkinElmer 1420 Multilabel Counter). As illustrated in FIG. 7B and Table 1, RP-043 does not bind to ANG-1, whereas VEGF-A/ANG-2/VEGF-C trispecific polypeptide RP-019 has a weak binding affinity to ANG-1.

TABLE 1
RP-043 and faricimab EC₅₀ binding affinity

| | Monospecific Target Binding (EC₅₀ nM) | | | | |
|---|---|---|---|---|---|
| | Biotin-aVpreB | | Biotin-aIgG-CH1 | | |
| | Detection | | Detection | | |
| Test | rhAng- | rhAng-1- | rhAng- | rhVEGF- | rhVEGF- |
| Protein | 2-cap | cap | 2-cap | A-cap | C-cap |
| RP-043 | 0.0667 | Not bind | 0.099 | 0.0189 | 0.0628 |
| Faricimab | Not done | Not done | 1.201 | 0.0250 | >10 |

Therefore, the tested trispecific RP-043 multispecific binding polypeptides showed a similar affinity to VEGF-A, higher affinity to ANG-2, and unique binding specificity to VEGF-C, when compared to the existing VEGF/ANG-2 bispecific antigen binding agent faricimab.

### Example 3—Binding of VEGF-A/ANG-2/VEGF-C Trispecific Antigen Binding Polypeptides to Both VEGF-A and ANG-2 or ANG-1 at the Same Time

The trispecific antigen binding polypeptide RP-043 prepared according to methods in example 1 were tested for their dual binding capacity to target antigens VEGF-A and ANG-2 (or ANG-1).

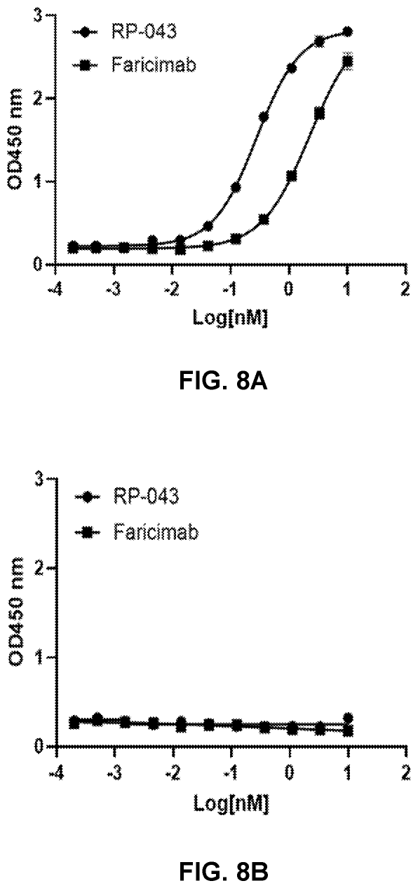

ANG-2-binding ELISA—Recombinant human ANG-2 (Acro Biosystems, Cat #AN2-H52H4) was used to coat plates at 1 μg/mL. The products were diluted in blocking buffer at a starting concentration of 10 nM and then 1:3 dilutions were tested. The binding was detected by 100 ng/mL biotinylated rhVEGF-A (Acro Biosystems, Cat #VE5-H8210) followed by 1:5,000 dilution of HRP-conjugated streptavidin (Jackson ImmuoResearch Lab, Cat #016-030-084). OD450 nm readings were measured by BioTek Synergy HTX Microplate Reader. As illustrated in FIG. 8A and Table 2, RP-043 has a much higher binding affinity with EC₅₀ value at 280 pM as compared to faricimab with EC₅₀ value at 2.28 nM.

ANG-1-binding ELISA—Recombinant human ANG-1 (R&D Systems Cat #923-AN-025/CF) was used to coat plates at 1 μg/mL. The products were diluted in blocking buffer at a starting concentration of 10 nM and then 1:3 dilutions were tested. The binding was detected by biotinylated rhVEGF-A (Acro Biosystems, Cat #VE5-H8210) followed by 1:5,000 dilution of HRP-conjugated streptavidin (Jackson ImmuoResearch Lab, Cat #016-030-084). OD450 nm readings were measured by OD450 nm readings were measured by BioTek Synergy HTX Microplate Reader. As illustrated in FIG. 8B and Table 2, neither RP-043 nor faricimab binds to ANG-1.

TABLE 2
RP-043 and faricimab dual binding affinity

| | |
|---|---|
| | Bispecific Target Binding |
| | (EC₅₀ nM) |
| | Bispecific |
| Test | (Biotin-VEGF-A-detection) |

| Protein | rhAng-2-cap | rhAng-1-cap |
|---|---|---|
| RP-043 | 0.2779 | Not bind |
| Faricimab | 2.284 | Not bind |

### Example 4—the Half Maximal Inhibitory Concentration (IC₅₀) of RP-043 VEGF-A/ANG-2/VEGF-C Trispecific Antigen Polypeptide

Trispecific antigen polypeptides RP-043 prepared according to example 1 were tested for their IC₅₀ of binding to target antigen. Inhibition of single target and receptor interaction binding ELISA was done using RP-043 and faricimab as the competitor, respectively.

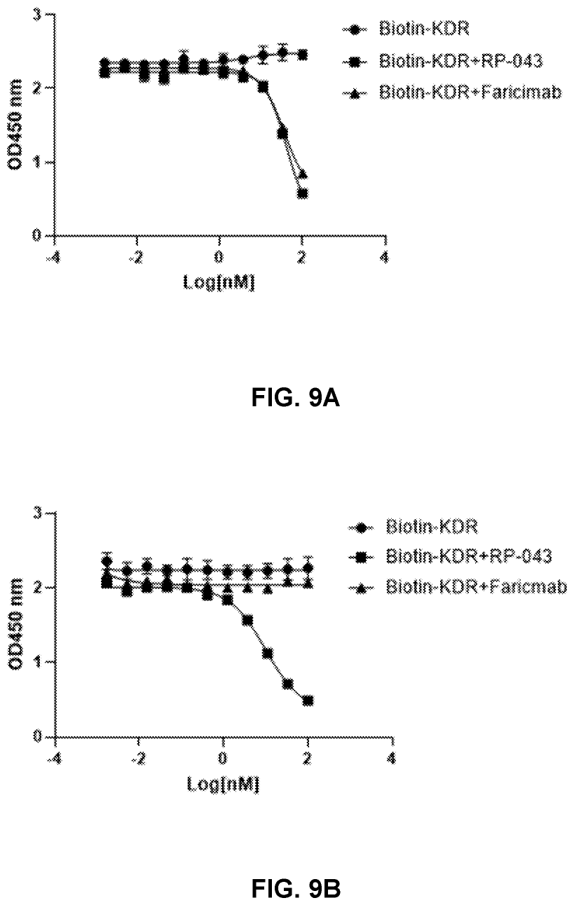

VEGF-A inhibition ELISA—The trispecific RP-043 were diluted in blocking buffer at a starting concentration of 200 nM and then 1:3 serial dilutions were tested. This 2× RP-043 dilutions were mixed with 2× biotinylated VEGF R2/KDR receptor (Acro Biosystems, Cat #VE5-H82Q0). Then 1× mixtures of RP-043 and biotinylated VEGF R2/KDR were added to plate wells. The binding was detected through a biotinylated receptor VEGF R2/KDR and 1:5,000 dilution of HRP-conjugated streptavidin (Jackson ImmunoResearch Lab, Cat #016-030-084). Faricimab was run as an inhibitor using the same protocol. OD450 nm readings were measured by BioTek Synergy HTX Microplate Reader As illustrated in FIG. 9A and Table 3, IC₅₀ of RP-043 was 40.66 nM, which was comparable to faricimab.

VEGF-C inhibition ELISA—Recombinant human_VEGF-C (Peprotech, Cat #100-20CD) was used to coat plates at 1 μg/mL. The trispecific RP-043 were diluted in blocking buffer at a starting concentration of 200 nM and then 1:3 serial dilutions were tested. This 2× RP-043 dilutions were mixed with 2× biotinylated VEGF R2/KDR receptor (Acro Biosystems, Cat #VE5-H82Q0). Then 1× mixtures of RP-043 and biotinylated VEGF R2/KDR were added to plate wells. The binding was detected through a biotinylated receptor VEGF R2/KDR and 1:5,000 dilution of HRP-conjugated streptavidin (Jackson ImmunoResearch Lab, Cat #016-030-084). Faricimab was run as an inhibitor using the same protocol. OD450 nm readings were measured by BioTek Synergy HTX Microplate Reader. As illustrated in FIG. 9B and Table 3, IC₅₀ of RP-043 was 9.82 nM and the inhibition to VEGF-C and receptor binding was unique to RP-043.

TABLE 3
The IC₅₀ value of RP-043 and faricimab
to VEGF/VEGF R2 interaction

| | Detected by Biotin-VEGF R2/KDR - IC50 (nM) | |
|---|---|---|
| Test Protein | rhVEGF-A-cap | rhVEGF-C-cap |
| Biotin-KDR | No inhibition | No inhibition |
| Biotin-KDR + | 36.84 | No inhibition |
| Faricimab | | |
| Biotin-KDR + RP-043 | 40.66 | 9.823 |

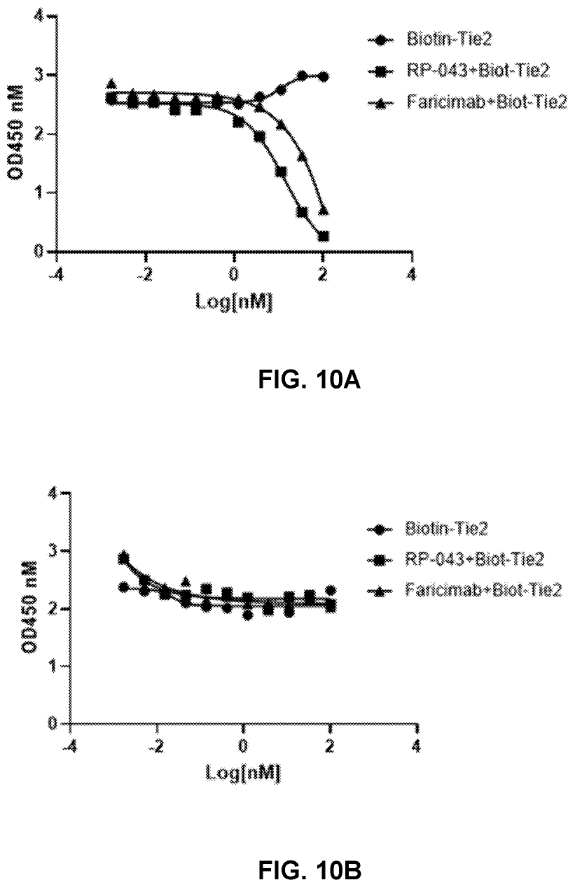

ANG-2 inhibition ELISA—Recombinant human_ANG-2 (Acro Biosystems, Cat #AN2-H52H4) was used to coat plates at 1 μg/mL. The trispecific RP-043 were diluted in blocking buffer at a starting concentration of 200 nM and then 1:3 serial dilutions were tested. This 2× RP-043 dilutions were mixed with 2× biotinylated Tie2 receptor (R&D Systems, Cat #AVI10727). Then 1× mixtures of RP-043 and biotinylated Tie2 were added to plate wells. The binding was detected through a biotinylated receptor Tie2 and 1:5,000 dilution of HRP-conjugated streptavidin (Jackson ImmunoResearch Lab, Cat #016-030-084). Faricimab was run as an inhibitor using the same protocol. OD450 nm readings were measured by Victor 3 (PerkinElmer 1420 Multilabel Counter). As illustrated in FIG. 10A and Table 4, IC₅₀ of RP-043 was 13.63 nM, and IC₅₀ of faricimab was between 30-100 nM.

ANG-1 inhibition ELISA—Recombinant human_ANG-1 (R&D Systems Cat #923-AN-025/CF) was used to coat plates at 1 μg/mL. The trispecific RP-043 were diluted in blocking buffer at a starting concentration of 200 nM and then 1:3 serial dilutions were tested. This 2× RP-043 dilutions were mixed with 2× biotinylated Tie2 receptor (R&D Systems, Cat #AVI10727). Then 1× mixtures of RP-043 and biotinylated Tie2 were added to plate wells. The binding was detected through a biotinylated receptor Tie2 and 1:5,000 dilution of HRP-conjugated streptavidin (Jackson ImmunoResearch Lab, Cat #016-030-084). Faricimab was run as an inhibitor using the same protocol. OD450 nm readings were measured by Victor 3 (PerkinElmer 1420 Multilabel Counter). As shown in FIG. 10B and Table 4, neither RP-043 nor faricimab inhibits ANG-1 binding to the Tie2 receptor.

TABLE 4
The IC₅₀ value of RP-043 and faricimab to ANG/Tie2 interaction

| | |
|---|---|
| Test | Biotin-Tie2 Detection - IC₅₀ (nM) |

| Protein | rhAng-2-cap | rhAng-1-cap |
|---|---|---|
| RP-043 | 13.63 | No inhibition |
| Faricimab | ~30-100 | No inhibition |

### Example 5—Inhibition of VEGF-A and VEGF-C Stimulated Pathways in VEGF Bioassay

Trispecific antigen polypeptides RP-043 prepared according to example 1 were tested for their IC₅₀ of inhibiting VEGF-A and VEGF-C stimulated pathways.

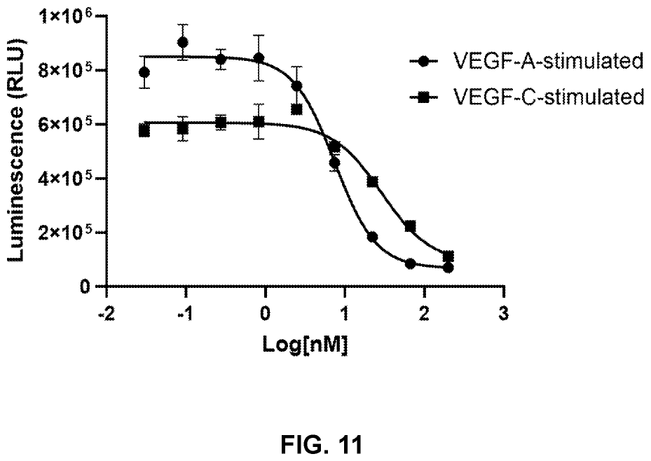

The trispecific RP-043 were diluted in buffer at a starting concentration of 200 nM and then 1:3 serial dilutions were tested in triplicates. The inhibition of VEGF bioassay either stimulated by rhVEGF-A (VEGF-A pathway) or rhVEGF-C (VEGF-C pathway) was carried out in Promega's VEGF R2/KDR-expressing HEK293 bioassay by following Promega's protocol. Briefly, 25 μL/well of KDR/NFAT-RE HEK293 cells were seeded to plate and 25 μL/well of 1:3 serial diluted 3× RP-043 starting at 600 nM was added. After cell stimulations with 25 μL of rhVEGF-A or rhVEGF-C stimulator, the Bio-Glo Reagent was added following Promega's instruction and the plates were read on GloMax (Promega) luminometer. The results were analyzed by GraphPad Prism. FIG. 11 and Table 5 show the IC₅₀ of VEGF-A and VEGF-C trispecific antigen binding polypeptide RP-043 were 7.451 nM and 15.95 nM, respectively.

TABLE 5
The IC₅₀ of RP-043 inhibition of VEGF-
A and VEGF-C stimulated pathways

| Bioassay | IC50 (nM) |
|---|---|
| VEGF-A | 7.451 |
| VEGF-C | 15.95 |

### Example 6—Inhibition of ANG-2 Stimulated Pathways in VEGF Bioassay

Trispecific antigen polypeptides RP-043 prepared according to example 1 were tested for their IC₅₀ of inhibiting ANG-2 stimulated pathways.

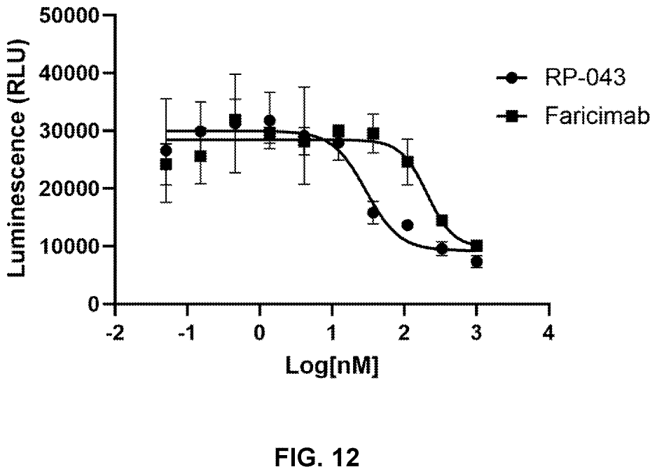

The inhibition of the trispecific RP-043 in Ang-2 bioassay was tested in the PathHunter HEK293 Tie2 Functional Assay (DiscoverX/Eurofin, Cat #93-1024C) by following DiscoverX/Eurofin's protocol. All cell culture media and detection kits were obtained from DiscoverX/Eurofin as described in the protocol of PathHunter HEK293 Tie2 Functional Assay. rhAng-2 was from Acro Biosystems (Cat #AN2-H52H4). Briefly, the cells were seeded on the day before assay for 24 hours. The trispecific RP-043 was serially diluted at 1:3 with a final starting concentration at 1 M and added to cells in triplicates, followed by the addition of rhAng-2 stimulator. After incubation, the detection reagents were added following DiscoverX/Eurofin's protocol. Plates were read and luminescence were measured by GloMax (Promega). Faricimab was tested as a control alongside. The results were analyzed for IC₅₀ by GraphPad Prism. As shown in Table 6 and FIG. 12, RP-043 inhibited ANG-2 interaction with Tie2 receptor with an IC₅₀ of 29.45 nM, which is seven-fold more potent than faricimab (IC50 206.2 nM).

TABLE 6
The IC₅₀ of RP-043 inhibition of ANG-
2 interaction with Tie2 receptor

| Bioassay | IC50 (nM) |
|---|---|
| RP-043 | 29.45 |
| Faricimab | 206.2 |

### Example 7-VEGF-A/ANG-2/VEGF-C Trispecific Antigen Binding Polypeptide RP-043 does not Inhibit Cynomolgus Monkey Tie2 Binding to ANG-1

Human ANG-1 sequence (SEQ ID NO:303) is 100% identical to one of the two reported cynomolgus monkey ANG-1 and 99.8% identical to the second cynomolgus monkey ANG-1 (SEQ ID NO: 304). VEGF-A/ANG-2/VEGF-C trispecific antigen-binding polypeptide RP-043 prepared according to example 1 were tested for its inhibition of cynomolgus monkey Tie2 binding to human ANG-1.

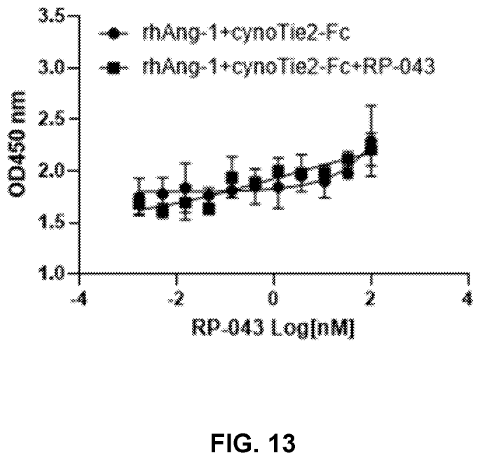

ANG-1 inhibition ELISA—Recombinant human_ANG-1 (R&D Systems Cat #923-AN-025/CF) was used to coat plates at 1 μg/mL. The trispecific RP-043 were diluted in blocking buffer at a starting concentration of 200 nM and then 1:3 serial dilutions were tested. This 2× RP-043 dilutions were mixed with 2× biotinylated cynomolgus monkey Tie2-hFc receptor (R&D Systems, Cat #10376-T2-100). Then 1× mixtures of RP-043 and biotinylated Tie2 were added to plate wells. The binding was detected through 1:5,000 dilution of HRP-conjugated donkey anti-human IgG-Fcγ (Jackson ImmunoResearch Lab, Cat #709-035-098). OD450 nm readings were measured by BioTek Synergy HTX Microplate Reader. As shown in FIG. 13, RP-043 does not inhibit ANG-1 binding to cynomolgus monkey Tie2 receptor.

### Example 8—Binding of VEGF-A/ANG-2/VEGF-C Trispecific Antigen Binding Polypeptide to VEGF-A, ANG-2, and VEGF-C from Different Species

VEGF-A/ANG-2/VEGF-C trispecific antigen-binding polypeptide RP-043 prepared according to example 1 were tested for their binding capacity to VEGF-A, ANG-2, and VEGF-C from different species.

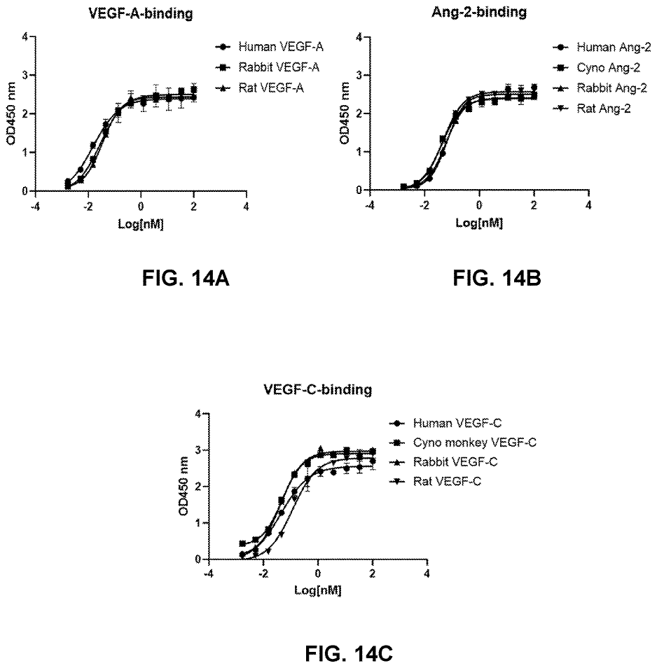

VEGF-A ELISA—Recombinant human (Peprotech, Cat #100-20), rabbit (Kingfisher, Cat #RP1025U), and rat (Sino Biological, Cat #80006-RNAB). VEGF-A were used to coat plates at 1 μg/mL. The products were diluted in blocking buffer at a starting concentration of 100 nM and then 1:3 dilutions were tested. The binding was detected by 100 ng/mL biotinylated anti-VpreB Ab followed by 1:5,000 dilution of HRP-conjugated streptavidin (Jackson ImmuoResearch Lab, Cat #016-030-084). OD450 nm readings were measured by BioTek Synergy HTX Microplate. As illustrated in FIG. 14A and Table 7, the results showed that the RP-043 antigen-binding polypeptide binds to VEGF-A from different species with comparable EC₅₀ between 15.3 pM and 34.6 pM.

ANG-2 ELISA—Recombinant human (Acro Biosystems, Cat #AN2-H52H4), monkey (Sino Biological, Cat #90026-C08H), rabbit (Speed Biosystems, Cat #YCP8015-100 ug), and rat (Acro Biosystems, Cat #AN2-R5244) ANG-2 were used to coat plates at 1 μg/mL. The products were diluted in blocking buffer at a starting concentration of 100 nM and then 1:3 dilutions were tested. The binding was detected by 100 ng/mL biotinylated anti-VpreB Ab followed by 1:5,000 dilution of HRP-conjugated streptavidin (Jackson ImmuoResearch Lab, Cat #016-030-084). OD450 nm readings were measured by BioTek Synergy HTX Microplate. As illustrated in FIG. 14B and Table 7, the results showed that the RP-043 antigen-binding polypeptide binds to ANG-2 from different species with comparable EC₅₀ between 41.1 pM and 65.2 pM.

VEGF-C ELISA—Recombinant human (Peprotech, Cat #100-20CD), monkey (made in house; SEQ ID NO: 301), rabbit (made in house; SEQ ID NO: 302), and rat (Sino Biological, Cat #80103-R08H) VEGF-C were used to coat plates at 1 μg/mL. The products were diluted in blocking buffer at a starting concentration of 100 nM and then 1:3 dilutions were tested. The binding was detected by 100 ng/mL biotinylated anti-VpreB Ab followed by 1:5,000 dilution of HRP-conjugated streptavidin (Jackson ImmuoResearch Lab, Cat #016-030-084). OD450 nm readings were measured by BioTek Synergy HTX Microplate. As illustrated in FIG. 14C and Table 7, the results showed that the RP-043 antigen-binding polypeptide binds to VEGF-C from different species with comparable EC₅₀ between 43.3 pM and 116.7 pM.

TABLE 7
EC₅₀ value of VEGF-A/ANG-2/VEGF-C trispecific polypeptides
to VEGF-A, ANG-2, and VEGF-C from different species

EC₅₀ (nM)

| X-species Target | VEGF-A | Ang-2 | VEGF-C |
|---|---|---|---|
| Human | 0.01525 | 0.0652 | 0.0433 |
| Cyno Monkey | Identical sequence | 0.0414 | 0.0490 |
| | to human | | |
| Rabbit | 0.0291 | 0.0592 | 0.0429 |
| Rat | 0.0346 | 0.0473 | 0.1167 |

### Example 9—Assessment of Pre-Existing Anti-Drug Antibodies (ADAs) Against RP-043 VEGF-A/ANG-2/VEGF-C Trispecific Antigen Polypeptide

Elevated levels of pre-existing anti-drug antibodies (ADAs) in patients may predict adverse outcomes in response to intravitreal injections with anti-vascular endothelial growth factor (anti-VEGF) agents. Higher levels of pre-existing ADAs detected against brolucizumab than ranibizumab may be correlated with a greater incidence of adverse events such as severe intraocular inflammation (101) including occlusive retinal vasculitis upon treatment with brolucizumab. The trispecific antigen polypeptide RP-043 prepared according to methods in example 1 were tested against pre-existing ADAs.

To detect ADAs, pooled human serum from Fisher Scientific (Cat #BP2657) was analyzed in an enzyme-linked immunosorbent assay (ELISA). Plate wells were coated with 100 μL of 1 μg/mL of the known most immunogenic agent (brolucizumab), the least immunogenic agent (ranibizumab), and our trispecific protein RP-043 made in 1× coating buffer (BioFx/Surmodics, Cat #COAT-1000-01). All plates were incubated overnight at room temperature. Wells were washed with PBS-Tween, blocked with 300 μL 1% BSA-PBST blocking buffer, washed three times again, and incubated with 100 μL of 1:3 serially diluted pooled human serum for 1 hour at room temperature. Samples were tested in duplicates. Wells were washed and then incubated with HRP-conjugated anti-human IgG-Fcγ secondary antibody (Jackson ImmunoResearch. Labs, Cat #709-035-098) for 1 hour at room temperature. After six washes, the TMB substrate and stop solution were added. ELISA results were visualized at OD450 nm by Biotek Synergy HTX plate reader. The ADA titer was determined as three-fold OD450 reading over background control.

As shown in Table 8, the ADA titer in pooled human serum against RP-043 was at least 1:2700. As a comparison, the ADA titers against brolucizumab was at least 1:8100, and the ADA titers against ranibizumab was at least 1:900. The result suggested that RP-043 have the lower amounts of pre-existing ADA in human serum than brolucizumab, and are therefore less likely than brolucizumab to induce adverse immunogenic responses in patients.

TABLE 8
Pre-existing ADA Titer in pooled human serum

| Test Protein | ADA Titer |
|---|---|
| Brolucizumab (most | >1:8100 |
| immunogenic) | |
| RP-043 | >1:2700 |
| Ranibizumab (Least | >1:900 |
| immunogenic) | |

### Example 10—Assessment of VEGF-A/ANG-2/VEGF-C Trispecific Antigen Polypeptide Target Binding Affinity by the Bio-Layer Interferometry Affinity Analysis

Trispecific antigen polypeptide RP-043 prepared according to example 1 were tested for their binding to target antigen to VEGF-A, ANG-2, VEGF-C, and ANG-1. Trispecific antigen polypeptides RP-019 was also prepared according to example 1 and run alongside RP-043.

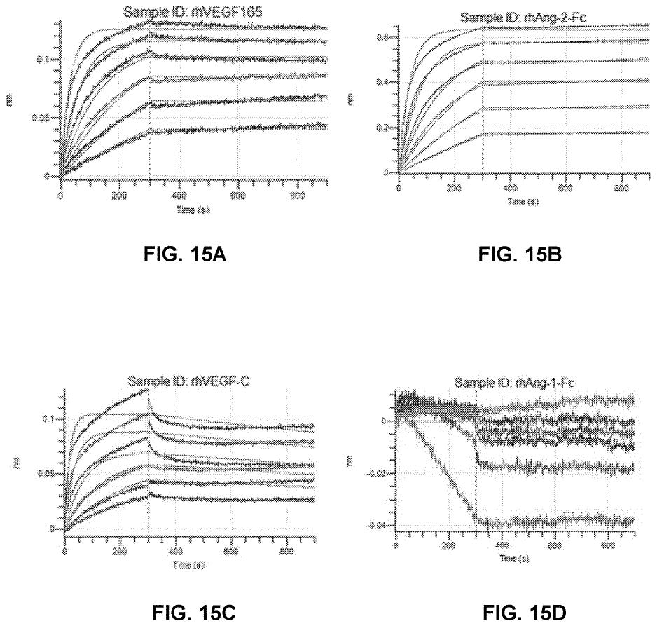

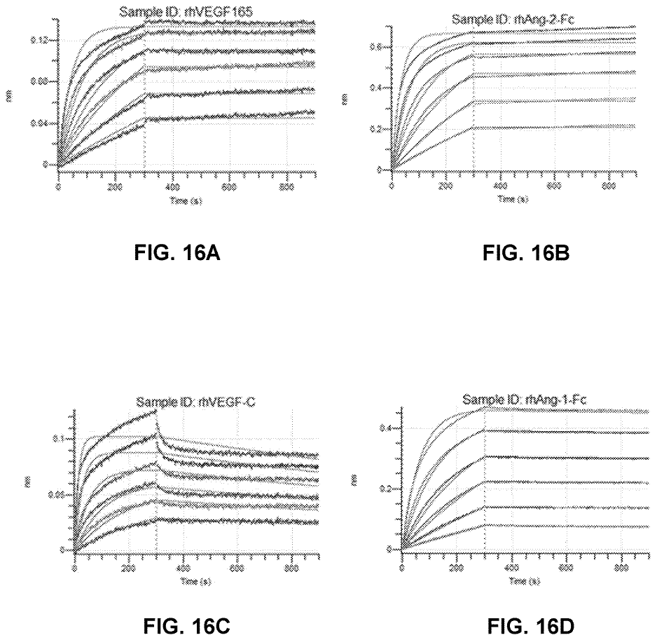

The Bio-Layer Interferometry (BLI) affinity analysis was carried out by Sino Biological using Octet R8 Protein Analysis System (Sartorius). RP-043 and control RP-019 were immobilized by Sartorius' streptavidin sensors via biotinylated anti-SLC/VpreB Ab and analyzed for affinities to rhVEGF165 (Sino Biological, Cat #11066-HNAH), rhAng-2-Fc (Sino Biological, Cat #10691-H02H), rhVEGF-C-His (Sino Biological, Cat #10542-H08H) and rhAng-1-Fc (Sino Biological, Cat #13667-H02H1). FIGS. 15A-15D show the target binding affinities of RP-043. FIGS. 16A-16D show the target binding affinities of RP-019. Table 9 summarizes the binding affinities of multispecific antigen binding polypeptides RP-043 and RP-019 to the targets and ANG-1. As shown in Table 9, both RP-043 and RP-019 showed strong binding affinity to VEGF-A and ANG-2 with a KD<1 pM. Both RP-043 and RP-019 have a strong binding affinity to VEGF-C with KD value of 497 pM and 484 pM, respectively. RP-019 binds to ANG-1 with a KD of 182 pM, whereas RP-043 does not bind to ANG-1.

TABLE 9
RP-043 and RP-019 binding affinity
to VEGF-A, VEGF-C, ANG-2, and ANG-1

| Target | | RP-043 | RP-019 |
|---|---|---|---|
| rhVEGF165 | KD (M) | <1.0E−12 | <1.0E−12 |
| (VEGF-A) | Kon (1/Ms) | 3.24E+05 | 2.31E+05 |
| | Kdis (1/s) | <1.0E−07 | <1.0E−07 |
| | Full R | 0.99 | 0.99 |
| rhVEGF-C | KD (M) | 4.97E−10 | 4.84E−10 |
| | Kon (1/Ms) | 7.44E+05 | 5.85E+05 |
| | Kdis (1/s) | 3.70E−04 | 2.86E−04 |
| | Full R | 0.97 | 0.96 |
| rhAng-2 | KD (M) | <1.0E−12 | <1.0E−12 |
| | Kon (1/Ms) | 2.58E+05 | 3.04+05 |
| | Kdis (1/s) | <1.0E−07 | <1.0E−07 |
| | Full R | 0.99 | 0.99 |
| rhAng-1 | KD (M) | — | 1.82E−10 |
| | Kon (1/Ms) | — | 1.59E+05 |
| | Kdis (1/s) | — | 2.9E−05 |
| | Full R | — | 1.00 |

### Example 11—Efficacy and Safety Analysis of RP-019 VEGF-A/ANG/2/VEGF-C Trispecific Antigen Polypeptide in Nonhuman Primate

Trispecific antigen polypeptides RP-019 (SEQ ID NO: 202) prepared according to example 1 was tested for their efficacy and safety in the nonhuman primate model of laser-induced choroidal neovascularization (LCNV).

Eight cynomolgus macaques received baseline retinal imaging followed by 9 LCNV lesions induced on the macula in both eyes on Day 0. Lesions were allowed to develop over a 14-day period and were then evaluated through a retinal exam and comprehensive retinal imaging including fluorescein angiography. Each lesion was then graded and assigned a score on a clinical severity scale on Day 14. On day 15, each eye received an intravitreal injection with animals divided into vehicle control (n=2), low dose VEGF-A/ANG-2/VEGF-C trispecific antigen polypeptide (0.27 mg; n=3), and high dose VEGF-A/ANG-2/VEGF-C trispecific antigen polypeptide (1.09 mg; n=3) groups. Two weeks later (Day 28), each animal received another retinal exam and additional retinal imaging, and every lesion was regraded. Clinical severity scores (grade 1—no hyperfluorescence to grade 4—clinically relevant leakage) were compared pre and post dosing across treatment groups. Mid-phase fluorescein angiogram images were also aligned using i2K retina, and pre and post dose lesion size was compared using ImageJ.

Pretreatment lesion grading resulted in 4% of lesions scored as Grade 1, 30% scored Grade 2, 19% scored Grade 3, and 47% scored Grade 4. Injection of vehicle, low (0.27 mg/eye), and high dose (1.09 mg/eye) was well tolerated. One animal in the low dose group experienced mild inflammation that self-resolved. Grade 1 and Grade 2 lesions remained without leakage.

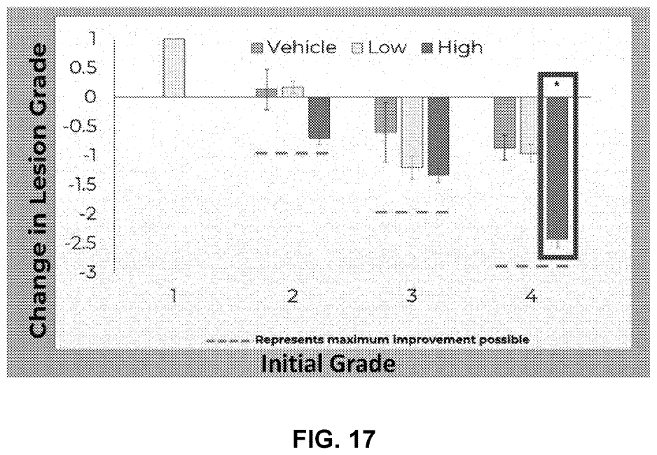

FIG. 17 shows changes in lesion grade after treatment in different grade groups. As shown in the figure, high dose treated eyes outperformed other treatments where on average Grade 4 lesions improved ~2.5 grades, Grade 3 lesions improved 1.5 grades, and Grade 2 lesions improved 1 grade. Low dose treated eyes outperformed vehicle controls in the Grade 3 lesion group with an average of 1 grade improvement. Lesion size was not different among treatment groups within initially scored Grade 1, 2, or 3 lesion scores. Both low and high dose treated eyes resulted in a 15-40% reduction in lesion area among Grade 4 lesions when compared to vehicle controls.

Overall, the result shows that VEGF-A/ANG-2/VEGF-C trispecific antigen polypeptide significantly reduced vascular leakage and lesion area when compared to controls in the non-human primate laser-induced choroidal neovascularization model.

### Example 12—In Vivo Anti-Angiogenic Efficacy of RP-043 in a Rat Model

Multispecific VEGF-A/ANG-2/VEGF-C antigen binding polypeptides RP-043 (SEQ ID NO: 201) were prepared according to example 1.

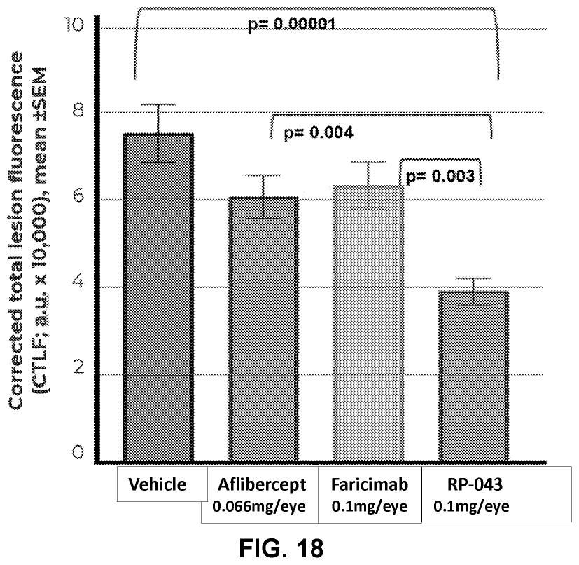

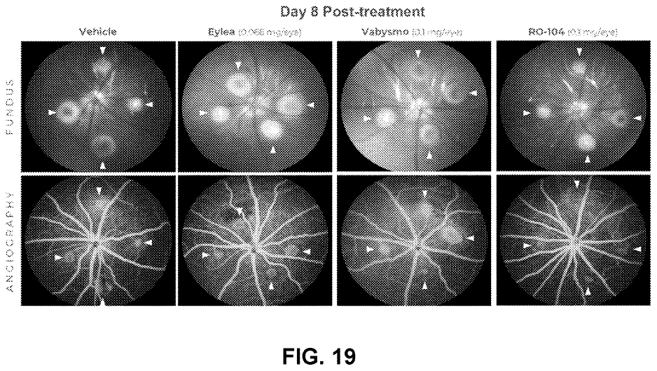

Rats were treated with laser to produce multiple Bruch's membrane injuries to induce neovascularized lesions. Following laser injury, rats were injected intravitreally with either vehicle, 0.1 mg of faricimab, 0.066 mg of aflibercept, or 0.1 mg of RP-043. After 8 days rats were intravenously injected with fluorescein and retinal vascular images were taken by fluoragraphical methods. FIG. 18 shows the mean corrected total lesion fluorescence (CTLF) in each group. FIG. 19 shows the representative retinal vascular images. Compared to the vehicle group, the rats treated with faricimab, and the rats treated with aflibercept, the CTLF in rats injected with RP-043 were significantly reduced. The result suggests that the trispecific RP-043 surpasses faricimab and aflibercept in effectively treating neovascular growth.

### Example 13—Efficacy and Safety Analysis of VEGF-A/ANG/2/VEGF-C Trispecific Antigen Polypeptide in Nonhuman Primate

Trispecific antigen polypeptides RP-043 (SEQ ID NO: 201) prepared according to example 1 is tested for their efficacy and safety in the nonhuman primate model of laser-induced choroidal neovascularization (LCNV).

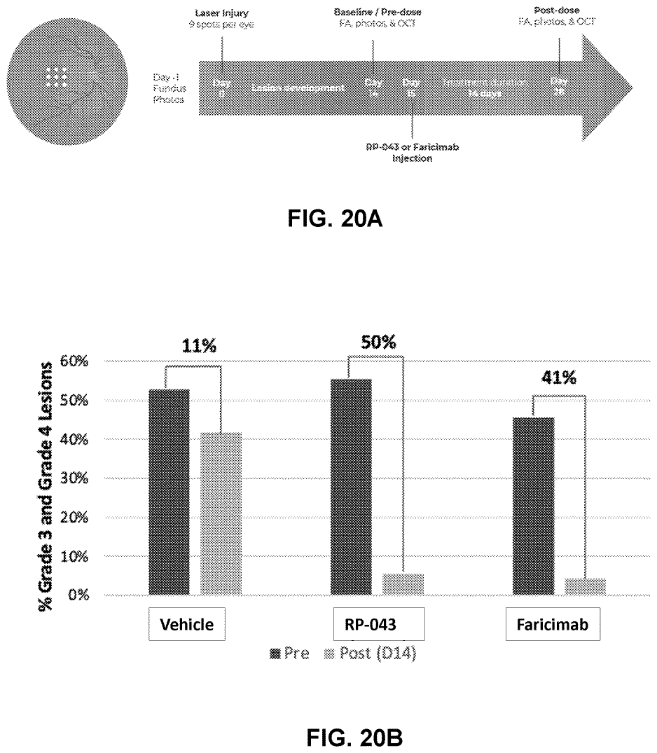

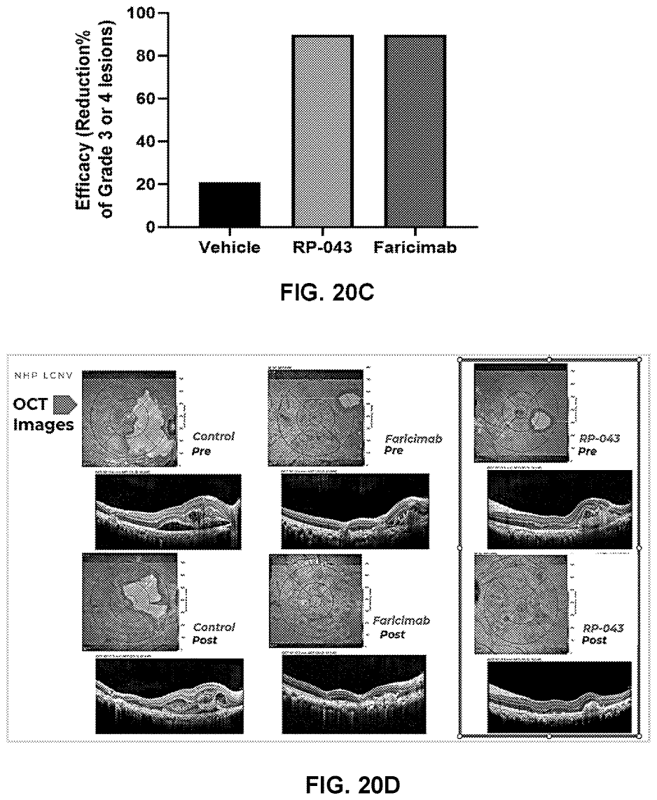

Twelve cynomolgus macaques received baseline retinal imaging followed by 9 LCNV lesions induced on the macula in both eyes on Day 0. Lesions were allowed to develop over a 14-day period and were then evaluated through a retinal exam and comprehensive retinal imaging including fluorescein angiography. Each lesion was then graded and assigned a score on a clinical severity scale on Day 14. On day 15, each eye received an intravitreal injection with animals divided into vehicle control (n=2), VEGF-A/ANG-2/VEGF-C RP-043 trispecific antigen polypeptide (1 mg; n=5), and faricimab (1 mg; n=5) groups. Two weeks later (Day 28), each animal received another retinal exam and additional retinal imaging, and every lesion was regraded. Clinical severity scores (grade 1—no hyperfluorescence; grade 2—hyperfluorescence, no leak; grade 3—hyperfluorescence, late leak; grade 4—bright hyperfluorescence with late leak beyond spot, clinically relevant) were compared pre and post dosing across treatment groups. Mid-phase fluorescein angiogram images were also aligned using i2K retina, and pre and post dose lesion size was compared using ImageJ. FIG. 20A illustrates the LCNV study protocol design.

Table 10 summarizes the frequency of laser lesions in different treatment groups. FIG. 20B shows changes in percentage of grade 3 and grade 4 lesions after treatment in different grade groups. FIG. 20C shows the efficacy in percentage reduction. FIG. 20D shows the pre-dose and post-dose OCT images of the eyes in different groups treated with RP-043, faricimab, or vehicle control. As shown in FIG. 20C, RP-043 demonstrated strong efficacy, producing a 90% reduction in clinically significant grade 3-4 FA lesions. The result demonstrated that VEGF-A/ANG-2/VEGF-C trispecific antigen polypeptide RP-043 significantly reduced high grade lesions in a non-human primate model and has comparable efficacy to faricimab. RP-043 also showed earlier anatomic resolution of laser lesions, with improvement observed by Day 1 compared to Day 8 for faricimab and minimal resolution in controls by Day 15. OCT imaging showed substantial post-dose resolution of pigment epithelial detachment and subretinal/intraretinal fluid in the RP-043 group.

TABLE 10
Anatomic resolution of laser spots on Indirect Ophthalmoscopy.
Frequency of Flattening of Laser Lesions on Indirect Ophthalmoscopy

| | Predose | Day 1 | Day 3 | Day 8 | Day 15 |
|---|---|---|---|---|---|
| Vehicle Control | — | — | — | — | 1 |
| Faricimab | — | — | — | 7 | 9 |
| RP-043 | — | 2 | 3 | 6 | 9 |

### Example 14—Pharmacokinetic Analysis of RP-043 in Rabbit Eye Vitreous

Trispecific antigen polypeptides RP-043 (SEQ ID NO: 201) prepared according to example 1 was tested for their pharmacokinetic profile in vivo.

Fourteen Dutch Belted Rabbits received a single intravitreal (IVT) injection of RP-043 at a dosage of 1 mg/eye in both eyes. Two Dutch Belted Rabbits received no test article as the controls. The concentration of RP-043 was measured across a 29-day period. To determine the concentration of RP-043 bound to rhVEGF-A in serum and ocular tissues (vitreous humor, aqueous humor, and retina), rabbits were sacrificed on each time points on days 1, 3, 6, 9, 12, 22 and 29. The collected retina tissues were homogenized, and the tissue debris were removed by centrifugation. All experiments were performed in accordance with the ARVO statement for Use of Animals in Ophthalmic and Vision Research.

Detection of RP-043 in the sample matrices was carried out in rhVEGF-binding ELISA. Serum samples, ocular tissue matrices or RP-043 standard were serially diluted, and the concentrations were determined by GraphPad Prism using the RP-043 standard. The PK profile was analyzed by Phoenix WinNonlin v8.5. Table 11 shows the PK parameters for each matrix.

TABLE 11
Mean pharmacokinetic parameters in Dutch Belted
Rabbits following a single IVT injection

| | Tₘₐₓ | Cₘₐₓ | AUCₗₐₛₜ | MRTₗₐₛₜ | T_{1/2} | Tₗₐₛₜ | Cₗₐₛₜ | AUC_{inf} |
|---|---|---|---|---|---|---|---|---|
| Matrix | (h) | (ng/mL) | (h*ng/mL) | (h) | (h) | (h) | (ng/mL) | (h*ng/mL) |
| Serum | 48 | 563 | 48,900 | 68.4 | NC | 120 | 417 | NC |
| AH | 72 | 24,800 | 4,060,000 | 112 | 95.3 | 264 | 6890 | 5,010,000 |
| Retina | 24 | 176,000 | 22,200,000 | 108 | NR | 264 | 37,400 | NR |
| VH | 24 | 724,000 | 80,600,000 | 95.7 | 82.2 | 264 | 97,400 | 92,900,000 |

NC = Not Calculated,
NR = Not Reported (not reported due to insufficient fit),
AH = Aqueous Humor,
VH = Vitreous Humor

### Example 15—Pharmacokinetic Analysis of Multidose of RP-043 in Nonhuman Primate

Trispecific antigen polypeptides RP-043 (SEQ ID NO: 201) prepared according to example 1 was tested for their pharmacokinetic profile in vivo.

RP-043 was administered in the right eye of the cynomolgus monkey via intravitreal injection on days 1, 29, and 57 with a dose of 0 (buffer), 0.5 mg/eye, 1.06 mg/eye, or 2.92 mg/eye. Each dosage group contained 2 cynomolgus monkeys, 1 male and 1 female. Blood samples were collected from all animals on Days 1, 29, and 57 at pre-dose and approximately 8, 24, 48, 96, 144, 240, and 336 hours post-dose. Ocular tissues were collected and processed on the terminal Day 71. The quantitation of rhVEGF-A-binding RP-043 was performed in binding ELISA, and the concentrations were determined by GraphPad Prism using the RP-043 standard. The PK profile was analyzed by Phoenix WinNonlin.

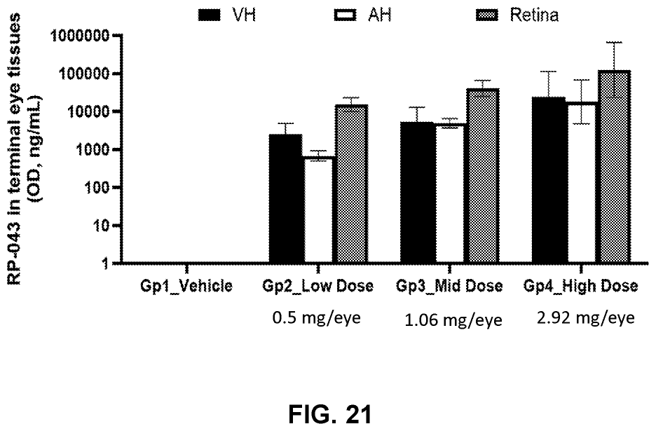

Table 12 summarizes the serum PK parameters. FIG. 21 shows the concentrations of VEGF-A in eye tissues on Day 14 post the 3^{rd} dosing. As shown in FIG. 21, RP-043 was detected at 14-days post the 3^{rd} IVT injection in vitreous humor (VH), aqueous humor (AH), and retina ocular tissues.

TABLE 12
Summary of the mean RP-043 TK parameters in monkey serum

| | | Dose | | | | | |
|---|---|---|---|---|---|---|---|
| | | Level | | | | | |
| Interval | Dose | (mg/eye/ | | Cₘₐₓ | Tₘₐₓ | AUC₀₋₃₃₆ | t_{1/2} |
| (Day) | Group | dose) | Sex | (ng/mL) | (h) | (h*ng/mL) | (h) |
| 1 | 2 | 0.5 | MF | 185 | 16.0 | 21200 | 71.0 |
| | 3 | 1.06 | MF | 625 | 8.00 | 30700 | 74.7 |
| | 4 | 2.92 | MF | 996 | 8.00 | 74100 | 80.4 |
| 29 | 2 | 0.5 | MF | 153 | 24.0 | 18200 | 71.8 |
| | 3 | 1.06 | MF | 321 | 16.0 | 39400 | 87.0 |
| | 4 | 2.92 | MF | 704 | 72.0 | 83400 | NA |
| 57 | 2 | 0.5 | MF | 124 | 24.0 | 16500 | 71.9 |
| | 3 | 1.06 | MF | 442 | 24.0 | 55500 | NA |

### Example 16—Exemplary Multispecific Antigen Binding Polypeptide Sequences

Exemplary sequences of the multispecific antigen binding polypeptides and its components are listed in Table 13.

TABLE 13
Exemplary sequences of the multispecific antigen binding polypeptides and its
components

| SEQ ID | | |
|---|---|---|
| NO. | Sequence | Construct |
| 1 | QVQLVQSGAEVKKPGASVKVSCKASGYTFSIYTIHWVR | anti-VEGFA- |
| | QAPGQGLEWMGGINPYNGNTKYAQKFQGRVTMTRDT | VH |
| | SISTAYMELSRLRSDDTAVYYCAKAPAVFWWTGLDYW | |
| | GQGTLVTVSS | |
| 2 | QVQLVQSGAEVKKPGASVKVSCKASGYTFSIYTIHWVR | anti-VEGFA- |
| | QAPGQGLEWMGGINPYNGNTKYAQKFQGRVTMTRDT | HC |
| | SISTAYMELSRLRSDDTAVYYCAKAPAVFWWTGLDYW | |
| | GQGTLVTVSSASTKGPSVFPLAPSSKSTSGGTAALGCLV | |
| | KDYFPEPVTVSWNSGALTSGVHTFPAVLQSSGLYSLSSV | |
| | VTVPSSSLGTQTYICNVNHKPSNTKVDKKVEPKSC | |
| 3 | SIYTIH | anti-VEGFA- |
| | | CDR1 |
| 4 | WMGGINPYNGNTK | anti-VEGFA- |
| | | CDR2 |
| 5 | AKAPAVFWWTGLD | anti-VEGFA- |
| | | CDR3 |
| 31 | IYTIH | anti-VEGFA- |
| | | CDR1 (Kabat) |
| 32 | GINPYNGNTKYAQKFQG | anti-VEGFA- |
| | | CDR2 (Kabat) |
| 33 | APAVFWWTGLDY | anti-VEGFA- |
| | | CDR3 (Kabat) |
| 34 | GYTFSIYT | anti-VEGFA- |
| | | CDR1 (IMGT) |
| 35 | INPYNGNT | anti-VEGFA- |
| | | CDR2 (IMGT) |
| 36 | AKAPAVFWWTGLDY | anti-VEGFA- |
| | | CDR3 (IMGT) |
| 37 | GYTFSIY | anti-VEGFA- |
| | | CDR1 |
| | | (Chothia) |
| 38 | NPYNGN | anti-VEGFA- |
| | | CDR2 |
| | | (Chothia) |
| 39 | APAVFWWTGLDY | anti-VEGFA- |
| | | CDR3 |
| | | (Chothia) |
| 11 | EVQLLESGGGLVQPGGSLRLSCAASGFTFSSFSMHWVR | anti-ANG-2- |
| | QAPGKGLEWVAGISGDGSDKNYADSVKGRFTISRDNSK | VH |
| | NTLYLQMNSLRAEDTAVYYCAKEFISYVYDRDWIDYW | |
| | GQGTLVTVSS | |
| 12 | EVQLLESGGGLVQPGGSLRLSCAASGFTFSSFSMHWVR | anti-ANG-2-HC |
| | QAPGKGLEWVAGISGDGSDKNYADSVKGRFTISRDNSK | |
| | NTLYLQMNSLRAEDTAVYYCAKEFISYVYDRDWIDYW | |
| | GQGTLVTVSSASTKGPSVFPLAPSSKSTSGGTAALGCLV | |
| | KDYFPEPVTVSWNSGALTSGVHTFPAVLQSSGLYSLSSV | |
| | VTVPSSSLGTQTYICNVNHKPSNTKVDKKVEPKSC | |
| 13 | SSFSMH | anti-ANG-2- |
| | | CDR1 |
| 14 | WVAGISGDGSDKN | anti-ANG-2- |
| | | CDR2 |
| 15 | AKEFISYVYDRDWID | anti-ANG-2- |
| | | CDR3 |
| 41 | SFSMH | anti-ANG-2- |
| | | CDR1 (Kabat) |
| 42 | GISGDGSDKNYADSVKG | anti-ANG-2- |
| | | CDR2 (Kabat) |
| 43 | EFISYVYDRDWIDY | anti-ANG-2- |
| | | CDR3 (Kabat) |
| 44 | GFTFSSFS | anti-ANG-2- |
| | | CDR1 (IMGT) |
| 45 | ISGDGSDK | anti-ANG-2- |
| | | CDR2 (IMGT) |
| 46 | AKEFISYVYDRDWIDY | anti-ANG-2- |
| | | CDR3 (IMGT) |
| 47 | GFTFSSF | anti-ANG-2- |
| | | CDR1 |
| | | (Chothia) |
| 48 | SGDGSD | anti-ANG-2- |
| | | CDR2 |
| | | (Chothia) |
| 49 | EFISYVYDRDWIDY | anti-ANG-2- |
| | | CDR3 |
| | | (Chothia) |
| 21 | EVQLLESGGGLVQPGGSLRLSCAASGFTFSSYGMSWVR | anti-VEGFC- |
| | QAPGKGLEWVARINGDGGYKNYADSVKGRFTISRDNS | VH |
| | KNTLYLQMNSLRAEDTAVYYCARELWAFINDFSFGLD | |
| | YWGQGTLVTVSS | |
| 22 | EVQLLESGGGLVQPGGSLRLSCAASGFTFSSYGMSWVR | anti-VEGFC- |
| | QAPGKGLEWVARINGDGGYKNYADSVKGRFTISRDNS | HC |
| | KNTLYLQMNSLRAEDTAVYYCARELWAFINDFSFGLD | |
| | YWGQGTLVTVSSASTKGPSVFPLAPSSKSTSGGTAALG | |
| | CLVKDYFPEPVTVSWNSGALTSGVHTFPAVLQSSGLYS | |
| | LSSVVTVPSSSLGTQTYICNVNHKPSNTKVDKKVEPKSC | |
| 23 | SSYGMS | anti-VEGFC- |
| | | CDR1 |
| 24 | WVARINGDGGYKN | anti-VEGFC- |
| | | CDR2 |
| 25 | ARELWAFINDFSFGLD | anti-VEGFC- |
| | | CDR3 |
| 51 | SYGMS | anti-VEGFC- |
| | | CDR1 (Kabat) |
| 52 | RINGDGGYKNYADSVKG | anti-VEGFC- |
| | | CDR2 (Kabat) |
| 53 | ELWAFINDFSFGLDY | anti-VEGFC- |
| | | CDR3 (Kabat) |
| 54 | GFTFSSYG | anti-VEGFC- |
| | | CDR1 (IMGT) |
| 55 | INGDGGYK | anti-VEGFC- |
| | | CDR2 (IMGT) |
| 56 | ARELWAFINDFSFGLDY | anti-VEGFC- |
| | | CDR3 (IMGT) |
| 57 | GFTFSSY | anti-VEGFC- |
| | | CDR1 |
| | | (Chothia) |
| 58 | NGDGGY | anti-VEGFC- |
| | | CDR2 |
| | | (Chothia) |
| 59 | ELWAFINDFSFGLDY | anti-VEGFC- |
| | | CDR3 |
| | | (Chothia) |
| 101 | QPVLHQPPAMSSALGTTIRLTCTLRNDHDIGVYSVYWY | VpreB |
| | QQRPGHPPRFLLRYFSQSDKSQGPQVPPRFSGSKDVARN | |
| | RGYLSISELQPEDEAMYYCAMGARSSVTHVFGSGTQLT | |
| | VL | |
| 102 | QPVLHQPPAMSSALGTTIRLTCTLRNDHDIGVYSVYWY | SLC-Fus1 |
| | QQRPGHPPRFLLRYFSQSDKSQGPQVPPRFSGSKDVARN | |
| | RGYLSISELQPEDEAMYYCAMGARSSVTHVFGSGTQLT | |
| | VLSQPKATPSVTLFPPSSEELQANKATLVCLMNDFYPGI | |
| | LTVTWKADGTPITQGVEMTTPSKQSNNKYAASSYLSLT | |
| | PEQWRSRRSYSCQVMHEGSTVEKTVAPAECS | |
| 103 | NDHDIGVYS | SLC-Fus1 |
| | | CDR1 |
| 104 | YFSQSDK | SLC-Fus1 |
| | | CDR2 |
| 105 | AMGARSSVTH | SLC-Fus1 |
| | | CDR3 |
| 106 | SQPKATPSVTLFPPSSEELQANKATLVCLMNDFYPGILT | 2.5 |
| | VTWKADGTPITQGVEMTTPSKQSNNKYAASSYLSLTPE | |
| | QWRSRRSYSCQVMHEGSTVEKTVAPAECS | |
| 141 | ASTKGPSVFPLAPSSKSTSGGTAALGCLVKDYFPEPVTV | CH1 |
| | SWNSGALTSGVHTFPAVLQSSGLYSLSSVVTVPSSSLGT | |
| | QTYICNVNHKPSNTKVDKKVEPKSC | |
| 151 | GGGGS | Linker |
| 201 | QVQLVQSGAEVKKPGASVKVSCKASGYTFSIYTIHWVR | Trispecific VA- |
| | QAPGQGLEWMGGINPYNGNTKYAQKFQGRVTMTRDT | A2-VC_HC |
| | SISTAYMELSRLRSDDTAVYYCAKAPAVFWWTGLDYW | (RP-043) |
| | GQGTLVTVSSASTKGPSVFPLAPSSKSTSGGTAALGCLV | |
| | KDYFPEPVTVSWNSGALTSGVHTFPAVLQSSGLYSLSSV | |
| | VTVPSSSLGTQTYICNVNHKPSNTKVDKKVEPKSCEVQ | |
| | LLESGGGLVQPGGSLRLSCAASGFTFSSFSMHWVRQAP | |
| | GKGLEWVAGISGDGSDKNYADSVKGRFTISRDNSKNTL | |
| | YLQMNSLRAEDTAVYYCAKEFISYVYDRDWIDYWGQG | |
| | TLVTVSSASTKGPSVFPLAPSSKSTSGGTAALGCLVKDY | |
| | FPEPVTVSWNSGALTSGVHTFPAVLQSSGLYSLSSVVTV | |
| | PSSSLGTQTYICNVNHKPSNTKVDKKVEPKSCEVQLLES | |
| | GGGLVQPGGSLRLSCAASGFTFSSYGMSWVRQAPGKG | |
| | LEWVARINGDGGYKNYADSVKGRFTISRDNSKNTLYL | |
| | QMNSLRAEDTAVYYCARELWAFINDFSFGLDYWGQGT | |
| | LVTVSSASTKGPSVFPLAPSSKSTSGGTAALGCLVKDYF | |
| | PEPVTVSWNSGALTSGVHTFPAVLQSSGLYSLSSVVTVP | |
| | SSSLGTQTYICNVNHKPSNTKVDKKVEPKSC | |
| 202 | QVQLVQSGAEVKKPGASVKVSCKASGYTFSIYTIHWVR | Trispecific VA- |
| | QAPGQGLEWMGGINPYNGNTKYAQKFQGRVTMTRDT | Ang2-VC_HC |
| | SISTAYMELSRLRSDDTAVYYCAKAPAVFWWTGLDYW | (RP-019) |
| | GQGTLVTVSSASTKGPSVFPLAPSSKSTSGGTAALGCLV | |
| | KDYFPEPVTVSWNSGALTSGVHTFPAVLQSSGLYSLSSV | |
| | VTVPSSSLGTQTYICNVNHKPSNTKVDKKVEPKSCEVQ | |
| | LVESGGGLVKPGGSLRLSCAASGFTFSSYGMHWVRQAP | |
| | GKGLEWVAGISADSGDKNYADSVKGRFTISRDNAKNSL | |
| | YLQMNSLRAEDTAVYYCAKEFISWIYTFDYLDYWGQG | |
| | TLVTVSSASTKGPSVFPLAPSSKSTSGGTAALGCLVKDY | |
| | FPEPVTVSWNSGALTSGVHTFPAVLQSSGLYSLSSVVTV | |
| | PSSSLGTQTYICNVNHKPSNTKVDKKVEPKSCEVQLLES | |
| | GGGLVQPGGSLRLSCAASGFTFSSYGMSWVRQAPGKG | |
| | LEWVARINGDGGYKNYADSVKGRFTISRDNSKNTLYL | |
| | QMNSLRAEDTAVYYCARELWAFINDFSFGLDYWGQGT | |
| | LVTVSSASTKGPSVFPLAPSSKSTSGGTAALGCLVKDYF | |
| | PEPVTVSWNSGALTSGVHTFPAVLQSSGLYSLSSVVTVP | |
| | SSSLGTQTYICNVNHKPSNTKVDKKVEPKSC | |
| 301 | AHYNAEILKSIDNEWRKTQCMPREVCIDVGKEFGVATN | Cynomolgus |
| | TFFKPPCVSVYRCGGCCNSEGLQCMNTSTSYLSKTLFEI | monkey VEGF-C |
| | TVPLSQGPKPVTISFANHTSCRCMSKLDVYRQVHSIIRR | (RP-078) |
| | AAAHHHHHHHH | |
| 302 | AHYNAEILKSIDNEWRKTQCMPREVCIDVGKEFGAATN | Rabbit VEGF-C |
| | TFFKPPCVSVYRCGGCCNSEGQQCMNTSTSYLSKTLFEI | (RP-079) |
| | TVPLSQGPKPVTISFANHTSCRCMSKLDVYRQVHSIIRR | |
| | AAAHHHHHHHH | |
| 303 | SNQRRSPENSGRRYNRIQHGQCAYTFILPEHDGNCREST | Mature Human |
| | TDQYNTNALQRDAPHVEPDFSSQKLQHLEHVMENYTQ | Ang-1 |
| | WLQKLENTIVENMKSEMAQIQQNAVQNHTATMLEIGT | |
| | SLLSQTAEQTRKLTDVETQVLNQTSRLEIQLLENSLSTY | |
| | KLEKQLLQQTNEILKIHEKNSLLEHKILEMEGKHKEELD | |
| | TLKEEKENLQGLVTRQTYIIQELEKQLNRATTNNSVLQK | |
| | QQLELMDTVHNLVNLCTKEGVLLKGGKREEEKPFRDC | |
| | ADVYQAGFNKSGIYTIYINNMPEPKKVFCNMDVNGGG | |
| | WTVIQHREDGSLDFQRGWKEYKMGFGNPSGEYWLGN | |
| | EFIFAITSQRQYMLRIELMDWEGNRAYSQYDRFHIGNEK | |
| | QNYRLYLKGHTGTAGKQSSLILHGADFSTKDADNDNC | |
| | MCKCALMLTGGWWFDACGPSNLNGIKWHYFKGPSYS | |
| | LRSTTMMIRPLDF | |
| 304 | SNQRRSPENSGRRYNRIQHGQCAYTFILPEHDGNCREST | Mature |
| | TDQYNTNALQRDAPHVEPDFSSQKLQHLEHVMENYTQ | Cynomolgus |
| | WLQKLENTIVENMKSEMAQIQQNAVQNHTATMLEIGT | monkey Ang-1 |
| | SLLSQTAEQTRKLTDVETQVLNQTSRLEIQLLENSLSTY | |
| | KLEKQLLQQTNEILKIHEKNSLLEHKILEMEGKHKEELD | |
| | TLKEEKENLQGLVTRQTYIIQELEKQLNRATTNNSVLQK | |
| | QQLELMDTVHNLVNLCTKEVLLKGGKREEEKPFRDCA | |
| | DVYQAGFNKSGIYTIYINNMPEPKKVFCNMDVNGGGW | |
| | TVIQHREDGSLDFQRGWKEYKMGFGNPSGEYWLGNEF | |
| | IFAITSQRQYMLRIELMDWEGNRAYSQYDRFHIGNEKQ | |
| | NYRLYLKGHTGTAGKQSSLILHGADFSTKDADNDNCM | |
| | CKCALMLTGGWWFDACGPSNLNGIKWHYFKGPSYSLR | |
| | STTMMIRPLDF | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

1.-54. (canceled)

55. An Angiopoietin-2 (ANG-2) binding moiety comprising an ANG-2 heavy chain variable region paired to a light chain polypeptide,
wherein the ANG-2 heavy chain variable region comprises:
(a) a heavy chain complementarity determining region 1 (HCDR1) comprising the amino acid sequence set forth in SEQ ID NO: 13; a heavy chain complementarity determining region 2 (HCDR2) comprising the amino acid sequence set forth in SEQ ID NO: 14; and a heavy chain complementarity determining region 3 (HCDR3) comprising the amino acid sequence set forth in SEQ ID NO: 15;
(b) a heavy chain complementarity determining region 1 (HCDR1) comprising the amino acid sequence set forth in SEQ ID NO: 41; a heavy chain complementarity determining region 2 (HCDR2) comprising the amino acid sequence set forth in SEQ ID NO: 42; and a heavy chain complementarity determining region 3 (HCDR3) comprising the amino acid sequence set forth in SEQ ID NO: 43;
(c) a heavy chain complementarity determining region 1 (HCDR1) comprising the amino acid sequence set forth in SEQ ID NO: 44; a heavy chain complementarity determining region 2 (HCDR2) comprising the amino acid sequence set forth in SEQ ID NO: 45; and a heavy chain complementarity determining region 3 (HCDR3) comprising the amino acid sequence set forth in SEQ ID NO: 46; or
(d) a heavy chain complementarity determining region 1 (HCDR1) comprising the amino acid sequence set forth in SEQ ID NO: 47; a heavy chain complementarity determining region 2 (HCDR2) comprising the amino acid sequence set forth in SEQ ID NO: 48; and a heavy chain complementarity determining region 3 (HCDR3) comprising the amino acid sequence set forth in SEQ ID NO: 49.

56. The ANG-2 binding moiety of claim 55, wherein the ANG-2 heavy chain variable region comprises an amino acid sequence comprising at least 85% identity to the amino acid sequence set forth in SEQ ID NO: 11.

57. The ANG-2 binding moiety of claim 55, wherein the ANG-2 heavy chain variable region comprises the amino acid sequence set forth in SEQ ID NO: 11.

58. The ANG-2 binding moiety of claim 55, wherein the light chain polypeptide comprises a surrogate light chain polypeptide, optionally comprising:
(a) a light chain complementarity determining region 1 (LCDR1) comprising the amino acid sequence set forth in SEQ ID NO: 103;
(b) a light chain complementarity determining region 2 (LCDR2) comprising the amino acid sequence set forth in SEQ ID NO: 104; and
(c) a light chain complementarity determining region 3 (LCDR3) comprising the amino acid sequence set forth in SEQ ID NO: 105.

59. The ANG-2 binding moiety of claim 58, wherein the surrogate light chain polypeptide comprises an amino acid sequence comprising at least 80% identity to the amino acid sequence set forth in SEQ ID NO: 102.

60. The ANG-2 binding moiety of claim 58, wherein the surrogate light chain polypeptide comprises the amino acid sequence set forth in SEQ ID NO: 102.

61. A multispecific antigen binding molecule comprising the ANG-2 binding moiety of claim 55, a VEGF-A binding moiety, and a VEGF-C binding moiety.

62. The multispecific antigen binding molecule of claim 61, wherein the VEGF-A binding moiety comprises a VEGF-A heavy chain variable region paired to a first surrogate light chain polypeptide, wherein the ANG-2 binding moiety comprises the ANG-2 heavy chain variable region paired to a second surrogate light chain polypeptide, wherein the VEGF-C binding moiety comprises a VEGF-C heavy chain variable region paired to a third surrogate light chain polypeptide,
wherein the VEGF-A heavy chain variable region comprises:
(a) a heavy chain complementarity determining region 1 (HCDR1) comprising the amino acid sequence set forth in SEQ ID NO: 3; a heavy chain complementarity determining region 2 (HCDR2) comprising the amino acid sequence set forth in SEQ ID NO: 4; and a heavy chain complementarity determining region 3 (HCDR3) comprising the amino acid sequence set forth in SEQ ID NO: 5;
(b) a heavy chain complementarity determining region 1 (HCDR1) comprising the amino acid sequence set forth in SEQ ID NO: 31; a heavy chain complementarity determining region 2 (HCDR2) comprising the amino acid sequence set forth in SEQ ID NO: 32; and a heavy chain complementarity determining region 3 (HCDR3) comprising the amino acid sequence set forth in SEQ ID NO: 33;
(c) a heavy chain complementarity determining region 1 (HCDR1) comprising the amino acid sequence set forth in SEQ ID NO: 34; a heavy chain complementarity determining region 2 (HCDR2) comprising the amino acid sequence set forth in SEQ ID NO: 35; and a heavy chain complementarity determining region 3 (HCDR3) comprising the amino acid sequence set forth in SEQ ID NO: 36; or
(d) a heavy chain complementarity determining region 1 (HCDR1) comprising the amino acid sequence set forth in SEQ ID NO: 37; a heavy chain complementarity determining region 2 (HCDR2) comprising the amino acid sequence set forth in SEQ ID NO: 38; and a heavy chain complementarity determining region 3 (HCDR3) comprising the amino acid sequence set forth in SEQ ID NO: 39,
wherein the VEGF-C heavy chain variable region comprises:
(a) a heavy chain complementarity determining region 1 (HCDR1) comprising the amino acid sequence set forth in SEQ ID NO: 23; a heavy chain complementarity determining region 2 (HCDR2) comprising the amino acid sequence set forth in SEQ ID NO: 24; and a heavy chain complementarity determining region 3 (HCDR3) comprising the amino acid sequence set forth in SEQ ID NO: 25;
(b) a heavy chain complementarity determining region 1 (HCDR1) comprising the amino acid sequence set forth in SEQ ID NO: 51; a heavy chain complementarity determining region 2 (HCDR2) comprising the amino acid sequence set forth in SEQ ID NO: 52; and a heavy chain complementarity determining region 3 (HCDR3) comprising the amino acid sequence set forth in SEQ ID NO: 53;
(c) a heavy chain complementarity determining region 1 (HCDR1) comprising the amino acid sequence set forth in SEQ ID NO: 54; a heavy chain complementarity determining region 2 (HCDR2) comprising the amino acid sequence set forth in SEQ ID NO: 55; and a heavy chain complementarity determining region 3 (HCDR3) comprising the amino acid sequence set forth in SEQ ID NO: 56; or
(d) a heavy chain complementarity determining region 1 (HCDR1) comprising the amino acid sequence set forth in SEQ ID NO: 57; a heavy chain complementarity determining region 2 (HCDR2) comprising the amino acid sequence set forth in SEQ ID NO: 58; and a heavy chain complementarity determining region 3 (HCDR3) comprising the amino acid sequence set forth in SEQ ID NO: 59.
and wherein the first, second, and third surrogate light chain polypeptides comprise:
(a) a light chain complementarity determining region 1 (LCDR1) comprising the amino acid sequence set forth in SEQ ID NO: 103;
(b) a light chain complementarity determining region 2 (LCDR2) comprising the amino acid sequence set forth in SEQ ID NO: 104; and
(c) a light chain complementarity determining region 3 (LCDR3) comprising the amino acid sequence set forth in SEQ ID NO: 105.

63. The multispecific antigen binding molecule of claim 62, wherein the VEGF-A heavy chain variable region comprises an amino acid sequence comprising at least 85% identity to the amino acid sequence set forth in SEQ ID NO: 1.

64. The multispecific antigen binding molecule of claim 62, wherein the VEGF-A heavy chain variable region comprises the amino acid sequence set forth in SEQ ID NO: 1.

65. The multispecific antigen binding molecule of claim 62, wherein the VEGF-C heavy chain variable region comprises an amino acid sequence comprising at least 85% identity to the amino acid sequence set forth in SEQ ID NO: 21.

66. The multispecific antigen binding molecule of claim 62, wherein the VEGF-C heavy chain variable region comprises the amino acid sequence set forth in SEQ ID NO: 21.

67. The multispecific antigen binding molecule of claim 62, wherein the first, second, and third surrogate light chain polypeptide comprises an amino acid sequence comprising at least 80% identity to the amino acid sequence set forth in SEQ ID NO: 102.

68. The multispecific antigen binding molecule of claim 62, wherein the surrogate light chain polypeptide comprises the amino acid sequence set forth in SEQ ID NO: 102.

69. The multispecific antigen binding molecule of claim 62, wherein the multispecific antigen binding molecule comprises an amino acid sequence comprising at least 85% identity to the amino acid sequence set forth in SEQ ID NO: 201.

70. The multispecific antigen binding molecule of claim 62, wherein the multispecific antigen binding polypeptide comprises the amino acid sequence set forth in SEQ ID NO: 201.

71. A nucleic acid or plurality of nucleic acids encoding the ANG-2 binding moiety of claim 55.

72. A pharmaceutical composition comprising the ANG-2 binding moiety of claim 55, and a pharmaceutically acceptable carrier, excipient, or diluent.

73. A method of treating an ocular disease in an individual comprising administering to the individual the ANG-2 binding moiety of claim 55, thereby treating the ocular disease.

74. A method of making the ANG-2 binding moiety of claim 55, comprising culturing a cell comprising a nucleic acid or plurality of nucleic acids encoding the ANG-2 binding antibody or antigen binding fragment thereof under conditions sufficient for expression of the nucleic acid and isolating or purifying the ANG-2 binding moiety or the multispecific antigen binding polypeptide or the antigen binding polypeptide from the cell or a supernatant there of.


SEQUENCE LISTING (304 sequences)

SEQ ID NO: 1 - protein, 121 aa, synthetic construct
QVQLVQSGAEVKKPGASVKVSCKASGYTFSIYTIHWVRQAPGQGLEWMGGINPYNGNTKYAQKFQGRVTMTRDTSISTAYMELSRLRSDDTAVYYCAKAPAVFWWTGLDYWGQGTLVTVSS

SEQ ID NO: 2 - protein, 224 aa, synthetic construct
QVQLVQSGAEVKKPGASVKVSCKASGYTFSIYTIHWVRQAPGQGLEWMGGINPYNGNTKYAQKFQGRVTMTRDTSISTAYMELSRLRSDDTAVYYCAKAPAVFWWTGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 3 - protein, 6 aa, synthetic construct
SIYTIH

SEQ ID NO: 4 - protein, 13 aa, synthetic construct
WMGGINPYNGNTK

SEQ ID NO: 5 - protein, 13 aa, synthetic construct
AKAPAVFWWTGLD

SEQ ID NO: 6-10 - skipped (5 entries)

SEQ ID NO: 11 - protein, 123 aa, synthetic construct
EVQLLESGGGLVQPGGSLRLSCAASGFTFSSFSMHWVRQAPGKGLEWVAGISGDGSDKNYADSVKGRFTISRDNSKNTLYLQMNSLRAEDTAVYYCAKEFISYVYDRDWIDYWGQGTLVTVSS

SEQ ID NO: 12 - protein, 226 aa, synthetic construct
EVQLLESGGGLVQPGGSLRLSCAASGFTFSSFSMHWVRQAPGKGLEWVAGISGDGSDKNYADSVKGRFTISRDNSKNTLYLQMNSLRAEDTAVYYCAKEFISYVYDRDWIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 13 - protein, 6 aa, synthetic construct
SSFSMH

SEQ ID NO: 14 - protein, 13 aa, synthetic construct
WVAGISGDGSDKN

SEQ ID NO: 15 - protein, 15 aa, synthetic construct
AKEFISYVYDRDWID

SEQ ID NO: 16-20 - skipped (5 entries)

SEQ ID NO: 21 - protein, 124 aa, synthetic construct
EVQLLESGGGLVQPGGSLRLSCAASGFTFSSYGMSWVRQAPGKGLEWVARINGDGGYKNYADSVKGRFTISRDNSKNTLYLQMNSLRAEDTAVYYCARELWAFINDFSFGLDYWGQGTLVTVSS

SEQ ID NO: 22 - protein, 227 aa, synthetic construct
EVQLLESGGGLVQPGGSLRLSCAASGFTFSSYGMSWVRQAPGKGLEWVARINGDGGYKNYADSVKGRFTISRDNSKNTLYLQMNSLRAEDTAVYYCARELWAFINDFSFGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 23 - protein, 6 aa, synthetic construct
SSYGMS

SEQ ID NO: 24 - protein, 13 aa, synthetic construct
WVARINGDGGYKN

SEQ ID NO: 25 - protein, 16 aa, synthetic construct
ARELWAFINDFSFGLD

SEQ ID NO: 26-30 - skipped (5 entries)

SEQ ID NO: 31 - protein, 5 aa, synthetic construct
IYTIH

SEQ ID NO: 32 - protein, 17 aa, synthetic construct
GINPYNGNTKYAQKFQG

SEQ ID NO: 33 - protein, 12 aa, synthetic construct
APAVFWWTGLDY

SEQ ID NO: 34 - protein, 8 aa, synthetic construct
GYTFSIYT

SEQ ID NO: 35 - protein, 8 aa, synthetic construct
INPYNGNT

SEQ ID NO: 36 - protein, 14 aa, synthetic construct
AKAPAVFWWTGLDY

SEQ ID NO: 37 - protein, 7 aa, synthetic construct
GYTFSIY

SEQ ID NO: 38 - protein, 6 aa, synthetic construct
NPYNGN

SEQ ID NO: 39 - protein, 12 aa, synthetic construct
APAVFWWTGLDY

SEQ ID NO: 40 - skipped

SEQ ID NO: 41 - protein, 5 aa, synthetic construct
SFSMH

SEQ ID NO: 42 - protein, 17 aa, synthetic construct
GISGDGSDKNYADSVKG

SEQ ID NO: 43 - protein, 14 aa, synthetic construct
EFISYVYDRDWIDY

SEQ ID NO: 44 - protein, 8 aa, synthetic construct
GFTFSSFS

SEQ ID NO: 45 - protein, 8 aa, synthetic construct
ISGDGSDK

SEQ ID NO: 46 - protein, 16 aa, synthetic construct
AKEFISYVYDRDWIDY

SEQ ID NO: 47 - protein, 7 aa, synthetic construct
GFTFSSF

SEQ ID NO: 48 - protein, 6 aa, synthetic construct
SGDGSD

SEQ ID NO: 49 - protein, 14 aa, synthetic construct
EFISYVYDRDWIDY

SEQ ID NO: 50 - skipped

SEQ ID NO: 51 - protein, 5 aa, synthetic construct
SYGMS

SEQ ID NO: 52 - protein, 17 aa, synthetic construct
RINGDGGYKNYADSVKG

SEQ ID NO: 53 - protein, 15 aa, synthetic construct
ELWAFINDFSFGLDY

SEQ ID NO: 54 - protein, 8 aa, synthetic construct
GFTFSSYG

SEQ ID NO: 55 - protein, 8 aa, synthetic construct
INGDGGYK

SEQ ID NO: 56 - protein, 17 aa, synthetic construct
ARELWAFINDFSFGLDY

SEQ ID NO: 57 - protein, 7 aa, synthetic construct
GFTFSSY

SEQ ID NO: 58 - protein, 6 aa, synthetic construct
NGDGGY

SEQ ID NO: 59 - protein, 15 aa, synthetic construct
ELWAFINDFSFGLDY

SEQ ID NO: 60-100 - skipped (41 entries)

SEQ ID NO: 101 - protein, 117 aa, synthetic construct
QPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVL

SEQ ID NO: 102 - protein, 223 aa, synthetic construct
QPVLHQPPAMSSALGTTIRLTCTLRNDHDIGVYSVYWYQQRPGHPPRFLLRYFSQSDKSQGPQVPPRFSGSKDVARNRGYLSISELQPEDEAMYYCAMGARSSVTHVFGSGTQLTVLSQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 103 - protein, 9 aa, synthetic construct
NDHDIGVYS

SEQ ID NO: 104 - protein, 7 aa, synthetic construct
YFSQSDK

SEQ ID NO: 105 - protein, 10 aa, synthetic construct
AMGARSSVTH

SEQ ID NO: 106 - protein, 106 aa, synthetic construct
SQPKATPSVTLFPPSSEELQANKATLVCLMNDFYPGILTVTWKADGTPITQGVEMTTPSKQSNNKYAASSYLSLTPEQWRSRRSYSCQVMHEGSTVEKTVAPAECS

SEQ ID NO: 107-140 - skipped (34 entries)

SEQ ID NO: 141 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 142-150 - skipped (9 entries)

SEQ ID NO: 151 - protein, 5 aa, synthetic construct
GGGGS

SEQ ID NO: 152-200 - skipped (49 entries)

SEQ ID NO: 201 - protein, 677 aa, synthetic construct
QVQLVQSGAEVKKPGASVKVSCKASGYTFSIYTIHWVRQAPGQGLEWMGGINPYNGNTKYAQKFQGRVTMTRDTSISTAYMELSRLRSDDTAVYYCAKAPAVFWWTGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEVQLLESGGGLVQPGGSLRLSCAASGFTFSSFSMHWVRQAPGKGLEWVAGISGDGSDKNYADSVKGRFTISRDNSKNTLYLQMNSLRAEDTAVYYCAKEFISYVYDRDWIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEVQLLESGGGLVQPGGSLRLSCAASGFTFSSYGMSWVRQAPGKGLEWVARINGDGGYKNYADSVKGRFTISRDNSKNTLYLQMNSLRAEDTAVYYCARELWAFINDFSFGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 202 - protein, 677 aa, synthetic construct
QVQLVQSGAEVKKPGASVKVSCKASGYTFSIYTIHWVRQAPGQGLEWMGGINPYNGNTKYAQKFQGRVTMTRDTSISTAYMELSRLRSDDTAVYYCAKAPAVFWWTGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEVQLVESGGGLVKPGGSLRLSCAASGFTFSSYGMHWVRQAPGKGLEWVAGISADSGDKNYADSVKGRFTISRDNAKNSLYLQMNSLRAEDTAVYYCAKEFISWIYTFDYLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEVQLLESGGGLVQPGGSLRLSCAASGFTFSSYGMSWVRQAPGKGLEWVARINGDGGYKNYADSVKGRFTISRDNSKNTLYLQMNSLRAEDTAVYYCARELWAFINDFSFGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 203-300 - skipped (98 entries)

SEQ ID NO: 301 - protein, 127 aa, synthetic construct
AHYNAEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRAAAHHHHHHHH

SEQ ID NO: 302 - protein, 127 aa, synthetic construct
AHYNAEILKSIDNEWRKTQCMPREVCIDVGKEFGAATNTFFKPPCVSVYRCGGCCNSEGQQCMNTSTSYLSKTLFEITVPLSQGPKPVTISFANHTSCRCMSKLDVYRQVHSIIRRAAAHHHHHHHH

SEQ ID NO: 303 - protein, 465 aa, Homo sapiens
SNQRRSPENSGRRYNRIQHGQCAYTFILPEHDGNCRESTTDQYNTNALQRDAPHVEPDFSSQKLQHLEHVMENYTQWLQKLENTIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEGVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGIKWHYFKGPSYSLRSTTMMIRPLDF

SEQ ID NO: 304 - protein, 464 aa, Macaca fascicularis
SNQRRSPENSGRRYNRIQHGQCAYTFILPEHDGNCRESTTDQYNTNALQRDAPHVEPDFSSQKLQHLEHVMENYTQWLQKLENTIVENMKSEMAQIQQNAVQNHTATMLEIGTSLLSQTAEQTRKLTDVETQVLNQTSRLEIQLLENSLSTYKLEKQLLQQTNEILKIHEKNSLLEHKILEMEGKHKEELDTLKEEKENLQGLVTRQTYIIQELEKQLNRATTNNSVLQKQQLELMDTVHNLVNLCTKEVLLKGGKREEEKPFRDCADVYQAGFNKSGIYTIYINNMPEPKKVFCNMDVNGGGWTVIQHREDGSLDFQRGWKEYKMGFGNPSGEYWLGNEFIFAITSQRQYMLRIELMDWEGNRAYSQYDRFHIGNEKQNYRLYLKGHTGTAGKQSSLILHGADFSTKDADNDNCMCKCALMLTGGWWFDACGPSNLNGIKWHYFKGPSYSLRSTTMMIRPLDF